SEQUENCE LISTING (66 sequences)

SEQ ID NO: 1 - RNA, 20 nt, synthetic construct
  source 1..20: mol_type = other RNA
  ncRNA 1..20: ncRNA_class = guide_RNA
gcagcatagtgagcccagaa

SEQ ID NO: 2 - RNA, 20 nt, synthetic construct
  source 1..20: mol_type = other RNA
  ncRNA 1..20: ncRNA_class = guide_RNA
tccttcttactgtccccttc

SEQ ID NO: 3 - RNA, 20 nt, synthetic construct
  source 1..20: mol_type = other RNA
  ncRNA 1..20: ncRNA_class = guide_RNA
ggcagcatagtgagcccaga

SEQ ID NO: 4 - RNA, 20 nt, synthetic construct
  source 1..20: mol_type = other RNA
  ncRNA 1..20: ncRNA_class = guide_RNA
cttgccccacagggcagtaa

SEQ ID NO: 5 - RNA, 20 nt, synthetic construct
  source 1..20: mol_type = other RNA
  ncRNA 1..20: ncRNA_class = guide_RNA
ggcaagaagcatggccaccg

SEQ ID NO: 6 - RNA, 20 nt, synthetic construct
  source 1..20: mol_type = other RNA
  ncRNA 1..20: ncRNA_class = guide_RNA
cttgtcaaggctattggtca

SEQ ID NO: 7 - RNA, 20 nt, synthetic construct
  source 1..20: mol_type = other RNA
  ncRNA 1..20: ncRNA_class = guide_RNA
acactgcagagatctcagct

SEQ ID NO: 8 - RNA, 20 nt, synthetic construct
  source 1..20: mol_type = other RNA
  ncRNA 1..20: ncRNA_class = guide_RNA
cacactgcagagatctcagc

SEQ ID NO: 9 - RNA, 20 nt, synthetic construct
  source 1..20: mol_type = other RNA
  ncRNA 1..20: ncRNA_class = guide_RNA
tggtaatgatggcttcaaca

SEQ ID NO: 10 - RNA, 20 nt, synthetic construct
  source 1..20: mol_type = other RNA
  ncRNA 1..20: ncRNA_class = guide_RNA
attcctgcagctgcccctgc

SEQ ID NO: 11 - RNA, 20 nt, synthetic construct
  source 1..20: mol_type = other RNA
  ncRNA 1..20: ncRNA_class = guide_RNA
cgacaattctgacgcccaat

SEQ ID NO: 12 - RNA, 20 nt, synthetic construct
  source 1..20: mol_type = other RNA
  ncRNA 1..20: ncRNA_class = guide_RNA
agctgcccctgctgggagtg

SEQ ID NO: 13 - RNA, 20 nt, synthetic construct
  source 1..20: mol_type = other RNA
  ncRNA 1..20: ncRNA_class = guide_RNA
gagaatcaaaatcggtgaat

SEQ ID NO: 14 - RNA, 20 nt, synthetic construct
  source 1..20: mol_type = other RNA
  ncRNA 1..20: ncRNA_class = guide_RNA
tctgtatctatattcatcat

SEQ ID NO: 15 - RNA, 20 nt, synthetic construct
  source 1..20: mol_type = other RNA
  ncRNA 1..20: ncRNA_class = guide_RNA
ttccagaggcgacctctgca

SEQ ID NO: 16 - DNA, 19 bp, synthetic construct
  source 1..19: mol_type = other DNA
gctgtatgaatccaggtcc

SEQ ID NO: 17 - DNA, 19 bp, synthetic construct
  source 1..19: mol_type = other DNA
cctcctggctgagaaaaag

SEQ ID NO: 18 - DNA, 26 bp, synthetic construct
  source 1..26: mol_type = other DNA
tgtttcctccaggataaggcagctgt

SEQ ID NO: 19 - DNA, 18 bp, synthetic construct
  source 1..18: mol_type = other DNA
gggaggattgggaagaca

SEQ ID NO: 20 - DNA, 20 bp, synthetic construct
  source 1..20: mol_type = other DNA
aggtgttcaggagaaggaca

SEQ ID NO: 21 - DNA, 25 bp, synthetic construct
  source 1..25: mol_type = other DNA
agcaggcatgctggggatgcggtgg

SEQ ID NO: 22 - DNA, 20 bp, synthetic construct
  source 1..20: mol_type = other DNA
gggaagacaatagcaggcat

SEQ ID NO: 23 - DNA, 20 bp, synthetic construct
  source 1..20: mol_type = other DNA
cgatcctgagacttccacac

SEQ ID NO: 24 - DNA, 24 bp, synthetic construct
  source 1..24: mol_type = other DNA
tggggatgcggtgggctctatggc

SEQ ID NO: 25 - DNA, 19 bp, synthetic construct
  source 1..19: mol_type = other DNA
agtccaagctgagcaaaga

SEQ ID NO: 26 - DNA, 20 bp, synthetic construct
  source 1..20: mol_type = other DNA
tagtgggaacgatgggggat

SEQ ID NO: 27 - DNA, 26 bp, synthetic construct
  source 1..26: mol_type = other DNA
cgagaagcgcgatcacatggtcctgc

SEQ ID NO: 28 - DNA, 20 bp, synthetic construct
  source 1..20: mol_type = other DNA
gctgcctatcagaaagtggt

SEQ ID NO: 29 - DNA, 19 bp, synthetic construct
  source 1..19: mol_type = other DNA
atcacaaacgcaggcagag

SEQ ID NO: 30 - DNA, 24 bp, synthetic construct
  source 1..24: mol_type = other DNA
ctggtgtggctaatgccctggccc

SEQ ID NO: 31 - DNA, 20 bp, synthetic construct
  source 1..20: mol_type = other DNA
gggtgaccaagtcaaggaag

SEQ ID NO: 32 - DNA, 20 bp, synthetic construct
  source 1..20: mol_type = other DNA
gatggtggtattcaagccga

SEQ ID NO: 33 - DNA, 29 bp, synthetic construct
  source 1..29: mol_type = other DNA
caagcgccatgttgaaacccagcctgccc

SEQ ID NO: 34 - DNA, 20 bp, synthetic construct
  source 1..20: mol_type = other DNA
gggaaggtaaaactggcaac

SEQ ID NO: 35 - DNA, 20 bp, synthetic construct
  source 1..20: mol_type = other DNA
gggcacatatacagctgtct

SEQ ID NO: 36 - DNA, 27 bp, synthetic construct
  source 1..27: mol_type = other DNA
cctcgccagtctcaacagggacccagc

SEQ ID NO: 37 - DNA, 22 bp, synthetic construct
  source 1..22: mol_type = other DNA
catgacattcatctgtggtggc

SEQ ID NO: 38 - DNA, 20 bp, synthetic construct
  source 1..20: mol_type = other DNA
tctcatttcgacaccgaagc

SEQ ID NO: 39 - DNA, 21 bp, synthetic construct
  source 1..21: mol_type = other DNA
gcacagggtggaacaagatgg

SEQ ID NO: 40 - DNA, 25 bp, synthetic construct
  source 1..25: mol_type = other DNA
ctcatagtgcatgttctttgtgggc

SEQ ID NO: 41 - DNA, 22 bp, synthetic construct
  source 1..22: mol_type = other DNA
ccagcccaggctgtgtatgaaa

SEQ ID NO: 42 - DNA, 23 bp, synthetic construct
  source 1..23: mol_type = other DNA
aggaagcagaactctgcacttca

SEQ ID NO: 43 - DNA, 27 bp, synthetic construct
  source 1..27: mol_type = other DNA
agtcagtgcctatcagaaacccaagag

SEQ ID NO: 44 - DNA, 21 bp, synthetic construct
  source 1..21: mol_type = other DNA
gagggagggctgagggtttga

SEQ ID NO: 45 - DNA, 20 bp, synthetic construct
  source 1..20: mol_type = other DNA
ccttctactcagttttagtc

SEQ ID NO: 46 - DNA, 21 bp, synthetic construct
  source 1..21: mol_type = other DNA
tgggtagtgtgaagggttcat

SEQ ID NO: 47 - DNA, 20 bp, synthetic construct
  source 1..20: mol_type = other DNA
aggcaagtgaatcctgagcg

SEQ ID NO: 48 - DNA, 21 bp, synthetic construct
  source 1..21: mol_type = other DNA
tgctttttgcgtcctggtgtt

SEQ ID NO: 49 - DNA, 21 bp, synthetic construct
  source 1..21: mol_type = other DNA
aacgcctgatcttgacagccc

SEQ ID NO: 50 - DNA, 18 bp, synthetic construct
  source 1..18: mol_type = other DNA
agatggcgccttcctcgc

SEQ ID NO: 51 - DNA, 20 bp, synthetic construct
  source 1..20: mol_type = other DNA
ctctcgcaggcactgaacat

SEQ ID NO: 52 - DNA, 24 bp, synthetic construct
  source 1..24: mol_type = other DNA
tcactttacctctcagaactgcac

SEQ ID NO: 53 - DNA, 21 bp, synthetic construct
  source 1..21: mol_type = other DNA
ggtgtgacctgccctgagctg

SEQ ID NO: 54 - DNA, 24 bp, synthetic construct
  source 1..24: mol_type = other DNA
aaccactgctaactgaaagagact

SEQ ID NO: 55 - DNA, 26 bp, synthetic construct
  source 1..26: mol_type = other DNA
gcactgaaactgttgctttataggat

SEQ ID NO: 56 - DNA, 24 bp, synthetic construct
  source 1..24: mol_type = other DNA
ggcgtctggactaggagcttattg

SEQ ID NO: 57 - DNA, 21 bp, synthetic construct
  source 1..21: mol_type = other DNA
aatgtcccacagtatccctgg

SEQ ID NO: 58 - DNA, 21 bp, synthetic construct
  source 1..21: mol_type = other DNA
agcccgtgtcacacagcacat

SEQ ID NO: 59 - DNA, 25 bp, synthetic construct
  source 1..25: mol_type = other DNA
cagcccagcttctgagttttatatg

SEQ ID NO: 60 - DNA, 22 bp, synthetic construct
  source 1..22: mol_type = other DNA
tgtgttgatgtcatccttgtcc

SEQ ID NO: 61 - DNA, 27 bp, synthetic construct
  source 1..27: mol_type = other DNA
cactgcatcagaatcatttggagaatc

SEQ ID NO: 62 - DNA, 24 bp, synthetic construct
  source 1..24: mol_type = other DNA
ggaaccatgggaagcatgtgatgt

SEQ ID NO: 63 - DNA, 20 bp, synthetic construct
  source 1..20: mol_type = other DNA
ctctgactcaccaactgggc

SEQ ID NO: 64 - DNA, 24 bp, synthetic construct
  source 1..24: mol_type = other DNA
gcgttttctcttctagggatctgc

SEQ ID NO: 65 - DNA, 33 bp, synthetic construct
  source 1..33: mol_type = other DNA
acactctttccctacacgacgctcttccgatct

SEQ ID NO: 66 - DNA, 32 bp, synthetic construct
  source 1..32: mol_type = other DNA
gactggagttcagacgtgtgctcttccgatct

(19) United States
(12) Patent Application Publication
(10) Pub. No.: US 2025/0305004 A1
(43) Pub. Date: Oct. 2, 2025

(54) ENHANCING GENE TARGETING EFFICIENCY IN HUMAN CELLS WITH DNA-PK INHIBITOR TREATMENT

(71) Applicant: The Board of Trustees of the Leland Stanford Junior University, Stanford, CA (US)

(72) Inventors: Sridhar Selvaraj, Stanford, CA (US); Matthew H. Porteus, Stanford, CA (US)

(73) Assignee: The Board of Trustees of the Leland Stanford Junior University, Stanford, CA (US)

(21) Appl. No.: 18/864,444

(22) PCT Filed: May 12, 2023
(86) PCT No.: PCT/US2023/022108

§ 371 Date: Nov. 8, 2024

Related U.S. Application Data
(60) Provisional application No. 63/341,683, filed on May 13, 2022.

(51) Int. Cl. C12N 15/90; C12N 9/22; C12N 15/11; C12N 15/86

(52) U.S. Cl. CPC C12N 15/907; C12N 9/226; C12N 15/11; C12N 15/86; C12N 2310/20; C12N 2310/315; C12N 2310/321; C12N 2501/727; C12N 2750/14143

(57) ABSTRACT

The present disclosure provides methods for enhancing the rate of homology-directed repair (HDR) during genomic editing in primary cells.

### CROSS-REFERENCE TO RELATED APPLICATIONS

This application claims priority to U.S. Provisional Application No. 63/341,683, filed May 13, 2022, the disclosure of which is herein incorporated by reference in its entirety for all purposes.

### BACKGROUND

CRISPR-Cas9 based gene editing technology has the potential for development of novel treatment methods for genetic diseases¹. Use of this technology to develop cell-based therapeutics using ex vivo gene edited cells to treat genetic diseases and cancer is being tested in clinical trials². Precise genetic modifications can be made using this technology by exploiting the endogenous DNA repair mechanisms. CRISPR-Cas9 based gene editing uses the Cas9 nuclease and the guide RNA (gRNA) specific to the targeted genomic loci to create a precise double stranded break (DSB)^{3,4}. This DSB is repaired by the cell either through non-homologous end joining (NHEJ) or homology-directed repair (HDR) pathways^{5,6}. NHEJ pathway processes the broken ends of DNA and ligates the ends together which may result in creation of short insertions and deletions (indels)⁷. This pathway can be used for genome editing to create gene knockouts, large deletions or targeted integration of foreign DNA. But, the frequency of NHEJ-based targeted integration of foreign DNA is quite low⁸. Alternatively, HDR pathway can be exploited for targeted integration of small or large DNA sequences by providing an exogenous donor template with the insert sequence flanked by homology arms⁴. In sum, genome editing harnesses endogenous DNA repair processes to generate precise genomic modifications.

Gene editing platforms involving the delivery of Cas9 and gRNA in the form of ribonucleoprotein (RNP) complex and donor template delivery through recombinant adeno associated virus 6 (AAV6) allows for highly efficient HDR-based gene targeting⁹. Using this platform, 20-60% gene targeting efficiencies can be achieved in various therapeutically relevant human primary cells such as pluripotent stem cells (PSCs)^{10,11}, hematopoietic stem and progenitor cells (HSPCs)^{9,12-14}, T cells¹⁵ and airway stem cells^{16,17}. HSCs have the ability to repopulate an entire hematopoietic system, and thus strategies aimed at developing cell-based therapies involving genome editing for various hematological diseases such as sickle cell disease, β-thalassemia, and X-linked severe combined immunodeficiency are progressing towards clinical trials. However, there is variability in gene targeting efficiencies across different genomic loci due to the inconsistency in the levels of HDR. Additionally, current xenograft studies support the idea that HSCs are more resistant to HDR-mediated editing, perhaps one mechanistic explanation for the observation that HDR-edited cells engraft less efficiently following transplantation in immunodeficient mice. Reductions in HDR frequency during long-term engraftment have been observed previously and therefore remains a major impediment to bringing HDR-mediated therapies to clinic⁸.

There exists therefore a need for new and efficient methods for promoting HDR-mediated genomic editing in primary cells, and particularly in hematopoietic stem cells (HSCs) or hematopoietic stem and progenitor cells (HSPCs). The present disclosure addresses these needs and provides other advantages as well.

### BRIEF SUMMARY

In one aspect, the present disclosure provides methods of genetically modifying a primary human cell, the methods comprising: introducing into the cell a site-directed nuclease (SDN) targeted to a cleavage site at a genetic locus of interest; introducing a homologous donor template into the cell, wherein the homologous donor template comprises a nucleotide sequence that is homologous to the locus of interest; and introducing a DNA-PK inhibitor into the cell; wherein the site-directed nuclease cleaves the locus at the cleavage site, and the homologous donor template is integrated at the site of the cleaved locus by homology directed repair (HDR).

In some embodiments, the DNA-PK inhibitor is a compound represented by the following formula:

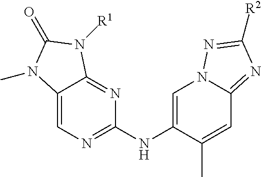

wherein R¹ is a cyclohexyl, tetrahydrofuranyl or oxanyl ring, each of which is optionally substituted by one or more groups selected from hydroxyl, methoxy, and methyl; and R² is hydrogen or methyl, or a pharmaceutically acceptable salt thereof. In some embodiments, R¹ is oxanyl. In some embodiments, R¹ is oxan-4-yl. In some embodiments, R² is hydrogen.

In some embodiments, the DNA-PK inhibitor is AZD7648 represented by the following formula:

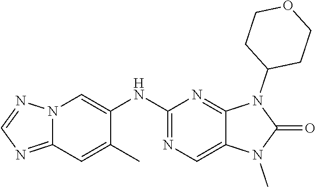 [SMILES: Cc1cc2ncnn2cc1Nc1ncc2c(n1)n(C1CCOCC1)c(=O)n2C]

or a pharmaceutically acceptable salt thereof.

In some embodiments, the DNA-PK inhibitor is VX984 represented by the following formula:

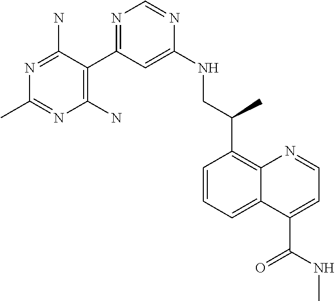

or a pharmaceutically acceptable salt thereof.

In some embodiments, the DNA-PK inhibitor is BAY8400 represented by the following formula:

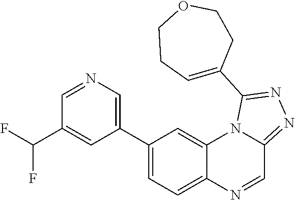 [SMILES: FC(F)c1cncc(-c2ccc3ncc4nnc(C5=CCCOCC5)n4c3c2)c1]

or a pharmaceutically acceptable salt thereof.

In some embodiments, the DNA-PK inhibitor has very high specificity for the catalytic subunit of DNA-PK (DNA-PKcs). In some embodiments, the DNA-PK inhibitor with very high specificity for DNA-PKcs has an IC50 in the range of about 40 nM to about 1 μM for DNA-PKcs and an IC50 of greater than 1 μM for other PIKK family kinases. In some embodiments, the other PIKK family kinases are ATM, ATR, PI3Kα, PI3Kβ, PI3Kγ, PI3Kδ, and/or mTOR.

In some embodiments of the methods provided herein, the SDN is an RNA-guided nuclease and the methods further comprise introducing into the cell a single guide RNA (sgRNA) targeting the cleavage site, wherein the sgRNA directs the RNA-guided nuclease to the cleavage site. In some embodiments, the sgRNA comprises 2′-O-methyl-3′-phosphorothioate (MS) modifications at one or more nucleotides. In some embodiments, the MS modifications are present at the terminal nucleotides of the 5′ and 3′ ends. In some embodiments, the RNA-guided nuclease is Cas9. In some embodiments, the sgRNA and RNA-guided nuclease are introduced into the cell as a ribonucleoprotein (RNP). In some embodiments, the RNP is introduced into the cell by electroporation. In some embodiments, the sgRNA is introduced into cells at a concentration of less than about 150 μg/ml, 75 μg/ml, 30 μg/ml, or 15 μg/ml. In some embodiments, the RNA-guided nuclease is introduced into cells at a concentration of less than about 300 μg/ml, 150 μg/ml, 60 μg/ml, or 30 μg/ml.

In some embodiments of the methods provided herein, the homologous repair template is introduced into the cell using an adeno-associated virus serotype 6 (AAV6) vector. In some embodiments, the AAV6 vector is transduced into the cell at a multiplicity of infection (MOI) of less than about 2500, 1000, or 500. IN some embodiments, the MOI is about 500.

In some embodiments of the methods provided herein, the primary human cell is a CD34+ hematopoietic stem and progenitor cell (HSPC), a T cell, a B cell, an airway basal stem cell, or a pluripotent stem cell (PSC). In some embodiments, the locus of interest is a gene selected from the group consisting of Hemoglobin Subunit Beta (HBB), C—C Motif Chemokine Receptor 5 (CCR5), Interleukin 2 Receptor Subunit Gamma (IL2RG), Hemoglobin Subunit Alpha 1 (HBA1), Stimulator Of Interferon Response cGAMP Interactor 1 (STING1) and Cystic Fibrosis Transmembrane Conductance Regulator (CFTR).

In some embodiments, the frequency of HDR at the locus of interest in the cell is higher than the frequency in an equivalent cell in the presence of the SDN and homologous donor template, but in the absence of the DNA-PK inhibitor. In some embodiments, the frequency of HDR at the locus of interest in the cell is at least about 10%, 20%, 30%, 40%, or more higher than the frequency in an equivalent cell in the presence of the SDN and homologous donor template, but in the absence of the DNA-PK inhibitor. In some embodiments, the sgRNA induces low to no indels at the locus of interest in the presence of the SDN but in the absence of the DNA-PK inhibitor. In some embodiments, the frequency of indels at the locus of interest in the cell is lower than the frequency in an equivalent cell in the presence of the SDN and homologous donor template, but in the absence of the DNA-PK inhibitor. In some embodiments, the frequency of indels at the locus of interest in the cell is at least about 10%, 20%, 30%, 40%, 50%, 60%, 70%, or more lower than the frequency in an equivalent cell in the presence of the SDN and homologous donor template, but in the absence of the DNA-PK inhibitor.

In some embodiments of the methods provided herein, the methods further comprise introducing a second SDN into the cell targeted to a second cleavage site at a second genetic locus, and introducing a second homologous donor template into the cell comprising a nucleotide sequence that is homologous to the second genetic locus, wherein the second SDN cleaves the second genetic locus at the second cleavage site, and the second homologous donor template is integrated at the site of the cleaved second locus by HDR. In some embodiments, the frequency of HDR is higher at both the locus of interest and the second genetic locus in the presence of the DNA-PK inhibitor than in the absence of the DNA-PK inhibitor. In some embodiments, the frequency of indels is lower at both the locus of interest and the second genetic locus in the presence of the DNA-PK inhibitor than in the absence of the DNA-PK inhibitor.

In some aspects, provided herein are methods of treating a genetic disorder in a human subject in need thereof, the methods comprising: providing an isolated primary cell from the subject; genetically modifying the primary cell using the methods of genetically modifying a primary human cell provided herein, wherein the integration of the homologous donor template at the locus of interest in the cell corrects a mutation at the locus or leads to the expression of a therapeutic protein in the cell that is absent or deficient in the subject; and reintroducing the genetically modified cell into the subject. In some embodiments, the genetic disorder is β-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof.

### BRIEF DESCRIPTION OF THE DRAWINGS

FIGS. 1A-1E show a comparison of different DNA-PK inhibitors for gene targeting at CCR5 locus and optimization in hPSCs, according to aspects of this disclosure. FIG. 1A: Schematic of gene targeting at CCR5 locus to introduce stop codons using RNP and AAV6.

FIG. 1B: Allelic gene targeting efficiency at CCR5 locus with and without treatment of AZD7648, M3814 and VX984 (1, 0.1 and 0.01 μM). ICE analysis was used to assess the gene targeting efficiency. FIG. 1C: Allelic gene at CCR5 locus with and without treatment of AZD7648, KU57788, BAY8400, LTURM34 (1, 0.1 and 0.01 μM). ICE analysis was used to assess the gene targeting efficiency. FIG. 1D: Allelic gene targeting efficiency at CCR5 locus following treatment with different concentrations of AZD7648 (1, 0.5, 0.25 and 0.1 μM) as measured by ICE analysis. FIG. 1E: Time course analysis of AZD-7648 treatment (0.25 μM for 4, 8, 12 and 24 h) on allelic gene targeting efficiency at CCR5 locus as measured by ICE analysis.

FIGS. 2A-2E show that AZD7648 enhances gene targeting efficiency for large sequence integrations at various genomic loci in hPSCs, according to aspects of this disclosure. FIG. 2A: Schematic of gene targeting strategy at CCR5 locus using RNP and AAV6 gene editing platform. FIG. 2B: Allelic gene targeting efficiency at CCR5 locus for integration of UBC-GFP-bGH-pA sequence with and without DNA-PK inhibitors, AZD7648, M3814, VX984 and BAY8400 (1, 0.1 and 0.01 μM) as measured by ddPCR analysis. FIG. 2C: Percentage of gene targeted cells with and without treatment of DNA-PK inhibitors, AZD7648, M3814, VX984 and BAY8400 (1, 0.1 and 0.01 μM) as measured by flow cytometry for GFP. FIG. 2D: Schematic of gene targeting strategy at HBB locus using RNP and AAV6 gene editing platform. FIG. 2F: Allelic gene targeting efficiency of UBC-GFP-bGH-PA sequence at HBB locus with and without AZD7648 and M3814 treatment (2 and 0.5 μM) as measured by ddPCR analysis. FIG. 2E: Schematic of gene targeting strategy at HBA1 locus using RNP and AAV6 gene editing platform. FIG. 2G: Allelic gene targeting efficiency of Transgene-2A-YFP sequence at HBA1 locus with and without AZD7648 and M3814 treatment (2 and 0.5 μM) as measured by ddPCR analysis.

FIGS. 3A-3F show that AZD7648 treatment enhances gene targeting efficiency for editing SCD and CF mutations in hPSCs, according to aspects of this disclosure. FIG. 3A: Schematic of gene targeting at HBB locus to correct SCD mutation (E6V) in exon 1 using RNP and AAV6. FIGS. 3B-3C: Allelic distribution of WT, INDEL and HR products (FIG. 3B) and allelic gene targeting efficiency (FIG. 3C) at HBB locus with or without AZD7648 and treatment (0.5 and 0.25 μM) as measured by ICE analysis. FIG. 3D: Schematic of gene targeting at CFTR locus to correct CF mutation (ΔF508) in exon 11 using RNP and AAV6.

FIGS. 3E-3F: Allelic distribution of WT, INDEL and HR products (FIG. 3E) and allelic gene targeting efficiency (FIG. 3F) at CFTR locus with or without AZD7648 and treatment (0.5 and 0.25 μM) as measured by ICE analysis.

FIGS. 4A-4E show that AZD7648 treatment enhances gene targeting efficiency at STING1 locus in hPSCs, according to aspects of this disclosure. FIG. 4A: Schematic of gene targeting at STING1 locus to introduce V155M mutation in exon 5 using RNP and AAV6.

FIGS. 4B-4C: Allelic distribution of WT, INDEL and HR products (FIG. 4B) and gene targeting efficiency (FIG. 4C) at STING1 locus with or without AZD7648 treatment (0.5 μM) using a seemingly inactive sgRNA as measured by ICE analysis. FIGS. 4D-4E: Allelic distribution of WT, INDEL and HR products (FIG. 4D) and gene targeting efficiency (FIG. 4E) at STING1 locus with or without AZD7648 treatment (0.5 and 0.25 μM) using an active sgRNA as measured by ICE analysis.

FIGS. 5A-5D show that gene targeting with AZD7648 treatment does not affect pluripotency and trilineage differentiation potential of hPSCs, according to aspects of this disclosure. FIG. 5A: Schematic of gene targeting strategy at CCR5 locus using RNP and AAV6 gene editing platform. FIG. 5B: Percentage of gene targeted cells with and without AZD7648 treatment (0.5 and 0.25 μM) as measured by flow cytometry for GFP. FIG. 5C: Pluripotency marker SSEA4 expression in gene targeted hPSCs as measured by flow cytometry analysis. FIG. 5D: Differentiation of gene targeted hPSC into three germ layers. Flow cytometry for relevant markers was used to measure the differentiation efficiency into Ectoderm (PAX6 and NES), Mesoderm (CD56 and T) and Endoderm (CXCR4 and SOX17).

FIGS. 6A-6E show gene targeting with AZD7648 with reduced amount of Cas9 RNP and AAV6 donor, according to aspects of this disclosure. FIG. 6A: Schematic of gene targeting strategy at CCR5 locus using RNP and AAV6 gene editing platform. FIGS. 6B-6C: Gene targeting using different amounts of Cas9 RNP (1, 0.5, 0.2 and 0.1×) with and without AZD7648 (AZD) treatment (0.25 μM). Allelic gene targeting efficiency (FIG. 6B) and percentage of targeted cells (FIG. 6C) was measured by ddPCR and flow cytometry for GFP respectively. FIGS. 6D-6E: Gene targeting using different amounts of AAV6 donor (MOI: 100, 500, 1000, 2500, 5000 and 10000) with and without AZD7648 (AZD) treatment (0.25 μM). Allelic gene targeting efficiency (FIG. 6D) and percentage of targeted cells (FIG. 6E) was measured by ddPCR and flow cytometry for GFP respectively.

FIGS. 7A-7C show that AZD7648 treatment enhances gene targeting efficiency in CB-CD34+ HSPC, according to aspects of this disclosure. FIG. 7A: Schematic of gene targeting strategy at HBB locus for SCD mutation using RNP and AAV6 gene editing platform (upper panel). Allelic distribution of WT, INDEL and HR products post gene targeting at HBB locus in CB-CD34+ HSPC with or without AZD7648 treatment (0.5 μM) (lower panel). FIG. 7B: Schematic for gene targeting strategy at CCR5 locus for integration of UBC-GFP-bGHpA using RNP and AAV6 gene editing platform (upper panel). Allelic gene targeting efficiency at CCR5 locus in CB-CD34+ HSPC with or without AZD7648 treatment (0.5 μM) (lower panel). FIG. 7C: Schematic for gene replacement at HBA1 locus to replace HBA1 with HBB sequence as a therapeutic strategy for β-thalassemia (upper panel). Allelic gene replacement efficiency at HBA1 locus in CB-CD34+ HSPC with or without AZD7648 treatment (0.5 μM) (lower panel).

FIGS. 8A-8C show that AZD7648 treatment enhances gene targeting efficiency in human T and B cells, according to aspects of this disclosure. FIG. 8A: Schematic of gene targeting strategy at CCR5 locus using RNP and AAV6 gene editing platform. FIG. 8B: Percentage of gene targeted cells post gene targeting with and without AZD7648 (0.5 μM) treatment at CCR5 locus in human T cells using different MOIs of the AAV6 donor (MOI: 1000, 2500, 5000 and 10000) as measured by flow cytometry. FIG. 8C: Allelic gene targeting efficiency at CCR5 locus with or without AZD7648 treatment (4, 2, 1, 0.5, 0.1 μM) in human B cells.

FIGS. 9A-9C show that AZD7648 treatment enhances gene targeting efficiency in human bronchial epithelial cells (HBECs). According to aspects of this disclosure. FIG. 9A: Schematic of gene targeting strategy at CFTR locus to correct AF508 mutation in exon 11 using RNP and AAV6 gene. FIG. 9B: Allelic distribution of WT, INDEL and HR products post gene targeting at CFTR locus in HBECs with or without AZD7648 treatment (0.5 μM) as measured by ICE analysis. FIG. 9C: Cell viability ratio of HBECs gene targeted with AZD7648 treatment (0.5 μM) relative to the untreated cells (gene targeted without AZD7648).

FIGS. 10a-10g: AZD7648 is the most potent DNA-PKcs inhibitor for improving gene targeting in PSCs. FIG. 10a. Table showing the half maximal inhibitory concentration (IC₅₀) values of different small molecule DNA-PKcs inhibitors (M3814, AZD7648 and VX984) against DNA-PKcs, ATM, ATR, mTOR and various PI3K family kinases in A549 cells (data adapted from previous study)³². FIG. 10b. Schematic of gene targeting strategy to introduce two stop codons at the CCR5 locus using Cas9 RNP and AAV6 gene editing. FIG. 10c. Allelic gene targeting efficiency post-gene editing at the CCR5 locus (FIG. 10b) with different concentrations (1, 0.1 and 0.01 μM) of DNA-PKcs inhibitors (AZD7648, M3814 and VX984) in comparison with the untreated cells (UNT) as measured by ICE analysis at 4 days post-gene editing (n=3). FIG. 10d. Concentration gradient analysis of AZD7648 treatment (1, 0.5, 0.25 and 0.1 μM) for gene targeting at the CCR5 locus (FIG. 10b). Percentage of targeted alleles was measured using ICE analysis at 4 days post-gene editing (n=3). FIG. 10e. Time course analysis of AZD7648 treatment (4 h, 8 h, 12 h and 24 h) for gene targeting at the CCR5 locus (FIG. 10b). Percentage of targeted alleles was measured using ICE analysis at 4 days post-gene editing (n=3). FIG. 10f. Schematic for gene targeting at the CCR5 locus for knock-in of UBC-GFP-bGHpA sequence using Cas9 RNP and AAV6 gene editing. FIG. 10g. Gene targeting at the CCR5 locus (FIG. 10f) with different concentrations (1, 0.1 and 0.01 μM) of DNA-PKcs inhibitors (AZD7648, M3814, VX984 and BAY8400). Percentage of targeted alleles was measured using ddPCR analysis at 5 days post-gene editing (n=3). All data are shown as mean±SEM. Data were compared with one-way ANOVA and Tukey's multiple comparisons test, *P<0.05, **P<0.01, ***P<0.001, ****P<0.0001 and ns denotes not significant.

FIGS. 11a-11h: AZD7648 improves gene targeting across different genomic loci in HSPCs. FIG. 11a. Allelic distribution of WT, INDEL and HDR frequencies following gene editing at the CCR5 locus for knock-in of UBC-GFP-bGHpA sequence using RNP/AAV6 gene editing with and without AZD7648 (0.5 μM) (n=6). HDR frequency was measured through ddPCR analysis. WT and INDEL frequencies were measured by ICE analysis. Mean HDR to INDEL ratio is represented above the bars. FIG. 11b. CFU assay was performed on Mock, RNP, AAV6 and RNP+AAV6 treated HSPCs following gene editing at the CCR5 locus with or without AZD7648 (0.5 μM) treatment. Plot shows the distribution of the absolute number of CFU-GEMM (multi-potential granulocyte, erythroid, macrophage, megakaryocyte progenitor cells), CFU-GM (colony forming unit-granulocytes and monocytes) and BFU-E (erythroid burst forming units) colonies (n=2). FIG. 11c. Single cell colonies (BFU-E, CFU-GM) from RNP+AAV6 treated cells with or without AZD7648 treatment from b were genotyped to assess the WT, INDEL and HDR frequencies. Plot shows the distribution of percentage of the BFU-E, CFU-GM colonies with different genotypes (n=1). FIG. 11d. Allelic distribution of WT, INDEL and HDR frequencies following gene editing at the CCR5 locus for knock-in of UBC-GFP-bGHpA sequence using RNP/AAV6 with and without AZD7648 (0.5 μM) in bulk, LT-HSC and MPP populations (n=2). HDR frequency was measured through ddPCR analysis. WT and INDEL frequencies were measured by ICE analysis. Mean HDR to INDEL ratio is represented above the bars. FIG. 11e. Allelic distribution of WT, INDEL and HDR frequencies following gene editing at the HBB locus for editing SCD mutation (E6V) using RNP/AAV6 with and without AZD7648 (0.5 μM) as measured by ICE analysis (n=4). Mean HDR to INDEL ratio is represented above the bars. FIG. 11f. Schematic for gene targeting at the HBG1/2 promoters to introduce a 13-bp deletion using Cas9 RNP and ssODN-based donor delivery. FIGS. 11g-11h. Allelic distribution of WT, 13-bp deletion and other INDEL frequencies at HBG1 (FIGS. 11g) and HBG2 (FIG. 11h) loci following gene editing (FIG. 11f) as measured by ICE analysis (n=2). ssODN was used at two different concentrations as indicated. AZD7648 was used at a concentration of 0.5 μM. Mean ratio of 13-bp deletion to other INDELs is represented above the bars. All data are shown as mean±SEM.

FIGS. 12a-12e: AZD7648 treatment improves gene targeting with seemingly inactive and low activity gRNAs. FIG. 12a. Schematic for gene targeting strategy at the STING1 locus (exon 5) to introduce a point mutation (V155M) associated with SAVI disease using RNP (sg3 and 5)/AAV6 gene editing. sg5 is a seemingly inactive gRNA and sg3 is an active gRNA FIG. 12b. Allelic distribution of WT, INDEL and HDR frequencies following gene editing at the STING1 locus (FIG. 12a) using sg3 and sg5 gRNAs in PSCs with and without AZD7648 treatment (0.5 μM) as measured by ICE analysis (n=3). Mean HDR to INDEL ratio is represented above the bars and ratio of ∞ denotes zero INDEL frequency. Nucleofection of sg5-Cas9 RNP alone resulted in only 1% of INDEL and thus the gRNA is designated seemingly inactive. FIG. 12c. Allelic distribution of WT, INDEL and HDR frequencies following STING1 gene editing using sg3 and sg5 gRNAs in human HSPCs with and without AZD7648 treatment (0.5 μM) as measured by ICE analysis (n=3). Mean HDR to INDEL ratio is represented above the bars and ratio of ∞ denotes zero INDEL frequency. FIG. 12d. PSCs were gene targeted at the CCR5 locus for the introduction of two stop codons using a high activity gRNA (sg11) and two low activity gRNAs (sg1 and 4). Allelic distribution of WT, INDEL and HDR frequencies following CCR5 gene editing using sg11, sg1 and sg4 gRNAs in human PSC with and without AZD7648 treatment (0.5 μM) as measured by ICE analysis (n=2). Mean HDR to INDEL ratio is represented above the bars. FIG. 12e. HSPCs were gene targeted at the CCR5 locus for the introduction of two stop codons using sg11, 1 and 4 gRNAs. Allelic distribution of WT, INDEL and HDR frequencies following CCR5 gene editing using sg11, sg1 and sg4 gRNAs in HSPCs with and without AZD7648 treatment (0.5 μM) as measured by ICE analysis (n=2). Mean HDR to INDEL ratio is represented above the bars. All data are shown as mean±SEM.

FIGS. 13a-13d: AZD7648 improves gene targeting with lower amounts of RNP and AAV6. FIG. 13a. Allelic distribution of WT, INDEL and HDR frequencies following gene editing at the CCR5 locus for knock-in of UBC-GFP-bGH-pA sequence in PSCs with or without AZD7648 treatment (0.25 μM) using varying amounts of Cas9-RNP as indicated and fixed amount of AAV6 donor (MOI. 2500) (n=3). HDR frequency was measured through ddPCR analysis. WT and INDEL frequencies were measured by ICE analysis. Mean HDR to INDEL ratio is represented above the bars. RNP-1× denotes 250 μg/ml of Cas9 protein complexed with 100 μg/ml of gRNA. RNP-0.5×, 0.2× and 0.1× denote 2-, 5-, and 10-fold lower concentrations of the RNP, respectively. FIG. 13b. Allelic distribution of WT, INDEL and HDR frequencies measured by ICE analysis following gene editing at the STING1 locus (sg3) in human HSPCs for introduction of V155M point mutation with or without AZD7648 treatment (0.5 μM) using varying amounts of Cas9-RNP as indicated and fixed amount of AAV6 donor (MOI:2500) (n=1). RNP-1× denotes 300 μg/ml of Cas9 protein complexed with 160 μg/ml of gRNA. Mean HDR to INDEL ratio is represented above the bars. FIG. 13c. Allelic distribution of WT, INDEL and HDR frequencies following gene editing at the CCR5 locus for knock-in of UBC-GFP-bGH-pA sequence in PSCs with or without AZD7648 treatment (0.25 μM) using a fixed amount of RNP and varying amounts of AAV6 donor (MOI: 100, 500, 1000, 2500, 5000 and 10000) (n=3). HDR frequency was measured through ddPCR analysis. WT and INDEL frequencies were measured by ICE analysis. Mean HDR to INDEL ratio is represented above the bars. FIG. 13d. Allelic distribution of WT, INDEL and HDR frequencies following gene editing at the CCR5 locus for knock-in of UBC-GFP-bGH-pA sequence in human HSPCs with or without AZD7648 treatment (0.5 μM) using a fixed amount of RNP and varying amounts of AAV6 donor (MOI: 100, 500, 1000, 2500 and 5000) (n=3). HDR frequency was measured through ddPCR analysis. WT and INDEL frequencies were measured by ICE analysis. Mean HDR to INDEL ratio is represented above the bars. All data are shown as mean±SEM.

FIGS. 14a-14g: AZD7648 improves gene targeting in primary human T cells and HBECs. FIG. 14a. Allelic distribution of WT, INDEL and HDR frequencies following gene editing at the CCR5 locus for knock-in of UBC-GFP-bGH-pA sequence in T cells with varying concentrations of AZD7648 (as indicated) using RNP/AAV6 gene editing (n=3). HDR frequency was measured by ddPCR. WT and INDEL frequencies were measured by ICE analysis. Mean HDR to INDEL ratio is represented above the bars. FIG. 14b. Allelic distribution of WT, INDEL and HDR frequencies following gene editing at the CCR5 locus for knock-in of UBC-GFP-bGH-pA sequence in T cells with or without AZD7648 (0.5 μM) using a fixed amount of RNP and varying amounts of AAV6 donor (MOI: 1000, 2500, 5000 and 10000) (n=3). FIG. 14c. Schematic for gene targeting at the TRAC gene locus for knock-in of CD19-CAR using RNP/AAV6 gene editing. FIG. 14d. Frequency of CD19-CAR positive cells measured by flow cytometry for tNGFR expression following gene editing at the TRAC gene locus (FIG. 14c) in T cells with or without AZD7648 (0.5 μM) treatment. Gene editing was performed with a fixed amount of RNP and two different MOIs of the AAV6 donor (n=4). Data were compared with unpaired t-test with Welch's correction, *P<0.05, ***P<0.001. FIG. 14e. Percentage of gene targeted alleles in WT HBECs post gene editing at the CFTR locus for editing AF508 mutation using RNP/AAV6 with different concentrations of AZD7648 as indicated (n=1). Allelic gene targeting frequency was measured using ICE analysis. FIG. 14f. Allelic distribution of WT, INDEL and HDR frequencies as measured by ICE analysis in CF patient-derived HBECs gene edited at the CFTR locus for the correction of AF508 mutation using RNP/AAV6 with or without AZD7648 treatment (0.5 μM) (n=4). Mean HDR to INDEL ratio is represented above the bars. FIG. 14g. Viable cell count of CFTR gene targeted HBECs with AZD7648 treatment (FIG. 14f) relative to that of untreated cells. All data are shown as mean±SEM.

FIGS. 15a-15d: Comparison of different DNA-PKcs inhibitors and optimization of gene targeting with AZD7648. FIG. 15a. Allelic gene targeting efficiency post gene editing at the CCR5 locus for introduction of stop codons with different concentrations of DNA-PKcs inhibitors (AZD7648, KU57788, LTURM34 and BAY8400) in comparison with the untreated cells (UNT) as measured by ICE analysis at 4 days post-gene editing (n=3). FIG. 15b. AZD7648 concentration gradient analysis in PSC for gene targeting at the CCR5 locus. Alignment of the WT and HDR sequences is shown in the top panel. Two stop codon sequences that are integrated by HDR is shown in green. Left panel shows representative sanger sequencing chromatograms for Mock, untreated (UNT, RNP+AAV6) and different concentrations of AZD7648 (1, 0.5, 0.25 and 0.1 μM) samples and the representative screenshots of ICE analysis data with WT, HDR and INDEL frequencies for corresponding samples is shown in the right panel. FIG. 15c. Time course analysis of AZD7648 treatment for gene targeting at the CCR5 locus in PSC. Alignment of the WT and HDR sequences is shown in the top panel. Two stop codon sequences that are integrated by HDR is shown in green. Left panel shows representative sanger sequencing chromatograms for Mock, untreated (UNT) and different time points of AZD7648 treatment (0.25 μM for 4 h, 8 h, 12 h and 24 h) samples and the screenshots of ICE analysis data with WT, HDR and INDEL frequencies for corresponding samples is shown on the right panel. FIG. 15d. Allelic gene targeting efficiency at the CCR5 locus for knock-in of UBC-GFP-bGHpA sequence with two different concentrations (0.5 and 0.25 μM) of various DNA-PKcs inhibitors (AZD7648, M3814, VX984 and BAY8400) as measured by ddPCR analysis at 5 days post-gene editing (n=3). All data are shown as mean±SEM. Data were compared with one-way ANOVA and Tukey's multiple comparisons test, *P<0.05, **P<0.01, ***P<0.001, ****P<0.0001 and ns denotes not significant.

FIGS. 16a-16d: Toxicity analysis in PSCs gene targeted with AZD7648 and biochemical validation of DNA-PKcs inhibition. FIG. 16a. MTT cell viability assay on gene targeted PSCs (FIG. 15d) at 24 h, 48 h and 72 h post-gene editing represented as percent cell viability normalized to the mock cells (n=3). All data are shown as mean±SEM. Data were compared with one-way ANOVA and Tukey's multiple comparisons test, *P<0.05, **P<0.01, ***P<0.001, ****P<0.0001 and ns denotes not significant. FIGS. 16b-16c. Western blot analysis for phosphop DNA-PKcs (Ser2056), DNA-PKcs and phospho-AKT (Ser473) in PSC treated with bleomycin+/−AZD for 2 h and gene targeted with and without AZD treatment at 2 h post gene editing (FIG. 16b). ACTB is used as a loading control. Control denotes sample not treated with bleomycin. Mock denotes control sample nucleofected without RNP. UNT denotes sample either treated with bleomycin or RNP-AAV6 gene editing and AZD denotes AZD7648 treated sample with either bleomycin or RNP-AAV6 gene editing treatment as indicated. Uncropped western blot images are shown in FIG. 16c. FIG. 16d. Allelic gene targeting efficiency of PSC gene targeted at the CCR5 locus for knock-in of UBC-GFP-bGHpA sequence (FIG. 16c) with and without AZD treatment at 72 h post gene editing as measured by ddPCR.

FIGS. 17a-17d: Gene targeting at CCR5 locus in PSC and pluripotency analysis. FIG. 17a. Percentage of gene targeted cells at the CCR5 locus for knock-in of UBC-GFP-bGHpA sequence with two different concentrations of AZD7648 (0.5 and 0.25 μM) in comparison with the untreated cells (UNT) as measured by flow cytometry for GFP at 5-days post-gene editing (n=3). Mock and RNP only cells were used as negative controls. FIG. 17b. Allelic distribution of WT, INDEL, HDR frequencies in CCR5 gene edited PSCs (FIG. 17a) (n=3). HDR frequency was measured by ddPCR analysis, WT and INDEL frequencies were measured using ICE analysis. Mean HDR to INDEL ratio is represented above the bars. FIG. 17c. Representative screenshots of ICE analysis in PSC gene targeted at CCR5 locus (FIG. 17a, FIG. 17b) showing frequencies of WT and INDELs along with the pattern. FIG. 17d. Percentage of cells positive for various markers of pluripotency (SSEA4, OCT3/4, SOX2 and NANOG) in the CCR5 gene edited PSCs (FIG. 17a) (n=3). SSEA4 expression was measured by flow cytometry analysis. OCT3/4, SOX2 and NANOG expression was assessed by quantification of immunofluorescence staining images for corresponding markers in fixed PSCs and normalized to the total cell count measured through DAPI staining (n=3).

FIGS. 18a-18d: Trilineage differentiation and single cell cloning of CCR5 gene targeted PSC. FIG. 18a. Gene edited PSCs (FIG. 17a) were differentiated into three germ layers using a commercially available differentiation kit. The frequency of differentiated cells was assessed by flow cytometry for the expression of corresponding markers for ectoderm (PAX6 and NES), mesoderm (CD56 and T) and endoderm (CXCR4 and SOX17) (n=2). FIG. 18b. PSCs were gene targeted at the CCR5 locus with and without different concentrations of AZD7648 (0.5, 0.25 and 0.1 μM) for single cell cloning analysis. Allelic gene targeting efficiency was measured using ddPCR analysis. FIG. 18c. Gene targeted PSCs (FIG. 18a) were subjected to single cell cloning and the frequency of clones with mono-, bi-allelic and no gene targeting was measured using ddPCR and PCR analysis. For each condition, 9-10 clones were picked and analyzed. FIG. 18d. Gene targeting in PSC at the CCR5 locus for the knock-in of UBC-GFP-bGHpA sequence with pre-treatment only (preAZD-UNT), post-treatment (AZD) only and pre+post treatment (pre+post AZD) with AZD7648 (0.5 μM) (n=2). For pre-treatment, cells were treated with AZD7648 for 24 hours before gene targeting and for post-treatment cells were treated with AZD7648 for 24 hours after gene editing.

FIGS. 19a-19d: AZD7648 treatment improves gene targeting at HBB and CFTR loci in PSC. FIG. 19a. Schematic for gene targeting at the HBB locus to edit SCD (E6V) mutation in exon 1 using RNP/AAV6 gene editing (upper panel). Allelic distribution of WT, INDEL and HDR frequencies following gene editing at the HBB locus with and without AZD7648 treatment as measured by ICE analysis (lower panel) (n=3). Mean HDR to INDEL ratio is represented above the bars. All data are shown as mean±SEM. FIG. 19b. Alignment of the WT and HDR sequences for HBB gene is shown in the top panel (FIG. 19a). Silent mutations in the HDR sequence are shown in green. Left panel shows representative sanger sequencing chromatograms for Mock, RNP, untreated (UNT, RNP+AAV6) and AZD7648 treatment (0.5, 0.25 μM) samples and the screenshots of ICE analysis data with WT, HDR and INDEL frequencies for corresponding samples is shown in the right panel. FIG. 19c. Schematic for gene targeting at the CFTR locus to edit CF disease mutation (AF508) in exon 11 using RNP/AAV6 gene editing (upper panel). Allelic distribution of WT, INDEL and HDR frequencies following gene editing with and without AZD7648 treatment at the CFTR locus as measured by ICE analysis (lower panel) (n=3). Mean HDR to INDEL ratio is represented above the bars. All data are shown as mean±SEM. FIG. 19d. Alignment of the WT and HDR sequences for CFTR gene (FIG. 19c) is shown in the top panel. Silent mutations in the HDR sequence are shown in green. Left panel shows representative sanger sequencing chromatograms for Mock, RNP, untreated (UNT, RNP+AAV6) and AZD7648 treatment (0.5, 0.25 μM) samples and the screenshots of ICE analysis data with WT, HDR and INDEL frequencies for corresponding samples is shown in the right panel.

FIGS. 20a-20d: AZD7648 treatment improves gene targeting at HBB and HBA1 loci in PSC. FIG. 20a. Schematic for gene targeting at the HBB locus to knock-in UBC-GFP-bGHpA sequence using RNP/AAV6 gene editing (upper panel). Allelic distribution of WT, INDEL and HDR frequencies following gene editing at the HBB locus with and without AZD7648 and M3814 (2 and 0.5 μM) treatment (lower panel) (n=3). HDR efficiency was measured through ddPCR analysis. WT and INDEL frequencies were measured by ICE analysis. Mean HDR to INDEL ratio is represented above the bars. All data are shown as mean±SEM. FIG. 20b. Representative screenshots of ICE analysis on PSC gene targeted at the HBB locus (FIG. 20a) showing frequencies of WT and INDELs along with the pattern. FIG. 20c. Schematic for gene replacement at the HBA1 locus for knock-in of transgene-2A-YFP sequence using RNP/AAV6 gene editing (upper panel). Allelic distribution of WT, INDEL and HDR frequencies following gene editing at the HBA1 locus with and without AZD7648 and M3814 (2 and 0.5 μM) treatment (lower panel) (n=3). HDR efficiency was measured through ddPCR analysis. WT and INDEL frequencies were measured by ICE analysis. Mean HDR to INDEL ratio is represented above the bars. All data are shown as mean±SEM. FIG. 20d. Representative screenshots of ICE analysis on PSC gene targeted at the HBA1 locus (FIG. 20c) showing frequencies of WT and INDELs along with the pattern.

FIGS. 21a-21d: Gene targeting in HSPCs at the CCR5 locus with AZD7648 treatment. FIG. 21a. Comparison of different DNA-PKcs inhibitors for gene targeting at the CCR5 locus for the knock-in of UBC-GFP-bGHpA sequence at two different concentrations (2 and 0.5 μM). Allelic gene targeting efficiency was measured by ddPCR analysis and cell viability was assessed by measuring live cell count and normalizing the number to the Mock sample (n=3). Mean values are shown above the bars. FIG. 21b. Allelic distribution of WT, INDEL and HDR frequencies following gene editing at the CCR5 locus for knock-in of UBC-GFP-bGHpA sequence with different concentrations of AZD7648 as indicated (n=1). HDR frequency was measured using ddPCR, WT and INDEL frequencies were measured using ICE analysis. FIG. 21c. Representative screenshots of ICE analysis on HSPCs gene targeted at the CCR5 locus (FIG. 21b) showing frequencies of WT and INDELs along with the pattern. FIG. 21d. CFU assay was performed on Mock, RNP, AAV6 and RNP+AAV6 treated HSPCs following gene editing at the CCR5 locus with or without AZD7648 (0.5 μM) treatment. Plot shows the distribution of the percentages of CFU-GEMM (multi-potential granulocyte, erythroid, macrophage, megakaryocyte progenitor cells), CFU-GM (colony forming unit-granulocytes and monocytes) and BFU-E (erythroid burst forming units) colonies (n=2). All data are shown as mean±SEM.

FIGS. 22a-22b: Gene targeting in LT-HSC and MPP with AZD7648 treatment. FIG. 22a. Gating scheme for FACS sorting of LT-HSC and MPP at 2 days post gene targeting of HSPCs at the CCR5 locus. LT-HSC (CD90+, CD45RA−, CD34+, CD38−, Lineage-) and MPP (CD90−, CD45RA−, CD34+, CD38−, Lineage-) populations were FACS sorted, and the gene targeting was assessed in these populations. FIG. 22b. FACS plots showing the frequency of GFP+ gene targeted cells in the Mock, AAV6 only, RNP+AAV6, RNP+AAV6+AZD samples in LT-HSC and MPP populations (FIG. 22a).

FIGS. 23a-23d: Gene targeting in HSPCs at HBB and HBA1 loci with AZD7648 treatment and cell viability. FIG. 23a. HSPCs were gene targeted at the HBB locus to edit SCD mutation using RNP/AAV6 gene editing with and without AZD7648 treatment (0.5 μM). Alignment of the WT and HDR sequences for HBB gene is shown in the top panel. Silent mutations in HDR sequence are shown in green. Left panel shows representative Sanger sequencing chromatograms for Mock, RNP, untreated (UNT, RNP+AAV6) and AZD7648 treatment (0.5 μM) samples and the screenshots of ICE analysis data with WT, HDR and INDEL frequencies for corresponding samples is shown in the right panel. FIG. 23b. Schematic for gene replacement at the HBA1 locus to replace HBA1 with HBB sequence using RNP/AAV6 gene editing (left panel). Allelic distribution of WT, INDEL and HDR frequencies following gene editing at the HBA1 locus with or without AZD7648 treatment (0.5 μM) (n=3) (right panel). HDR efficiency was measured through ddPCR analysis. WT and INDEL frequencies were measured by ICE analysis. Mean HDR to INDEL ratio is represented above the bars. FIG. 23c. Representative screenshots of ICE analysis on HSPC gene targeted at HBA1 locus (FIG. 23b) showing frequencies of WT and INDELs along with the pattern. FIG. 23d. Percentage of viable cell count of HSPCs at 3 days post-gene targeting (GT) at CCR5 (n=4), HBB (n=2) and HBA1 (n=2) loci in untreated (UNT) and AZD7648 (AZD, 0.5 μM) treated cells. Mock, AAV only and RNP only treated cells were included as controls. Viable cell counts were measured at 72 h post-gene editing and plotted as percentage relative to the mock cell count. All data are shown as mean±SEM.

FIGS. 24a-24c: Off-target analysis in gene targeted HSPC and AZD7648 treatment for gene editing without donor template. FIG. 24a. HSPCs gene targeted at HBB, CCR5 and HBA1 loci with and without AZD7648 treatment (0.5 μM) were assessed for off-target activity at the top off-target sites (OT1 for HBB, OT39 for CCR5 and OT1 for HBA1) through next generation sequencing. Frequency of reads with insertion and deletion INDELs is shown following SNP/INDEL detection analysis of NGS data (n=2). FIG. 24b. HSPC were gene edited with Cas9 RNP at the CCR5 locus with and without AZD7648 treatment in the presence or absence of AAV6 donor template (for knock-in of two stop codons). Allelic distribution of frequencies of WT, insertions, deletions and HDR were determined by using ICE analysis (n=2). FIG. 24c. Alignment of the WT and HDR sequences for the CCR5 gene (FIG. 24b) is shown in the top panel. Left panel shows representative sanger sequencing chromatograms for Mock, RNP only, RNP+AAV6 (untreated (UNT) and AZD7648 treated (AZD-0.5 μM)) samples and the screenshots of ICE analysis data with WT, HDR and INDEL frequencies for corresponding samples is shown in the right panel. All data are shown as mean±SEM.

FIGS. 25a-25f: AZD7648 treatment for gene editing without AAV6 donor template and ssODN based gene targeting at the HBB locus. FIG. 25a. HSPCs were gene edited with Cas9 RNP at the HBB locus with and without AZD7648 treatment in the presence or absence of AAV6 donor template (for SCD mutation editing). Allelic distribution of frequencies of WT, insertions, deletions and HDR were determined by using ICE analysis. FIGS. 25b-25c. Alignment of the WT and HDR sequences for the HBB gene (FIG. 25a) is shown in the top panel. Bottom panel shows representative sanger sequencing chromatograms for Mock, RNP only, RNP+AAV6 (untreated (UNT) and AZD7648 treated (AZD-0.5 μM)) samples and the screenshots of ICE analysis data with WT, HDR and INDEL frequencies for corresponding samples is shown in (FIG. 25c). FIG. 25d. Allelic distribution of WT, INDEL and HDR frequencies following gene editing at the HBB locus for editing SCD mutation using RNP and ssODN donor with and without AZD7648 treatment (0.5 μM) (n=2). ssODN donor was tested at two different concentrations as indicated (2.5 and 5 μM). FIGS. 25e-25f. Representative sanger sequencing chromatograms for Mock, RNP only, RNP+ssODN (untreated (UNT) and AZD7648 treated (AZD-0.5 μM)) samples (FIG. 25e) and the screenshots of ICE analysis data with WT, HDR and INDEL frequencies for corresponding samples is shown in (FIG. 25f). All data are shown as mean±SEM.

FIGS. 26a-26c: Gene targeting in HSPCs at HBG1/2 loci using ssODN donor with AZD7648 treatment. FIG. 26a. HSPCs were gene edited at HBG1/2 loci for introducing a 13-bp deletion using RNP and ssODN donor (2.5 and 5 μM) with and without AZD7648 treatment (0.5 μM). Alignment of the WT and HDR sequences for HBG1/2 genes is shown in the top panel. Bottom panels show representative sanger sequencing chromatograms of HBG1 (left) and HBG2 (right) loci for Mock, RNP only, RNP+ssODN (untreated (UNT) and AZD7648 treated (AZD-0.5 μM)) samples. FIGS. 26b-26c. Screenshots of ICE analysis data with WT, 13-bp deletion and other INDEL frequencies for the corresponding samples from (FIG. 26a) is shown for HBG1 (FIG. 26b) and HBG1 (FIG. 26c).

FIGS. 27a-27c: Gene targeting at the STING1 locus in PSC and HSPC using seemingly inactive gRNA with AZD7648 treatment. FIG. 27a. Alignment of the WT and HDR sequences for the STING1 gene. HDR introduces V155M mutation (in red). Silent mutations introduced by HDR are indicated in green. FIGS. 27b-27c. PSC and HSPC were gene targeted at STING1 locus with an active (sg3) and inactive (sg5) gRNA. Left panel shows representative Sanger sequencing chromatograms for Mock, sg3, sg5-RNP only, RNP+AAV6 (untreated (UNT) and AZD7648 treated (AZD-0.5 μM)) samples in PSC (FIG. 27b) and HSPC (FIG. 27c). Screenshots of ICE analysis data with WT, HDR and INDEL frequencies for the corresponding samples from (FIGS. 27b-27c) is shown in the right panel.

FIGS. 28a-28d: Gene targeting at the CCR5 locus in PSCs and HSPCs using seemingly low activity gRNAs with AZD7648 treatment. FIG. 28a. Target sites for sg11, sg1 and sg4 gRNAs at the CCR5 locus. sg11 is a highly active gRNA and sg1, 4 are low activity gRNAs FIG. 28b. Alignment of the WT and HDR sequences for the CCR5 gene. HDR introduces two stop codons (in green). FIGS. 28c-28d. PSCs and HSPCs were gene targeted at the CCR5 locus with sg11, sg1 and sg4 gRNAs. Left panel shows representative Sanger sequencing chromatograms for Mock, sg11, sg1, sg4-RNP only, RNP+AAV6 (untreated (UNT) and AZD7648 treated (AZD-0.5 μM)) samples in PSCs (FIG. 28c) and HSPCs (FIG. 28d). Screenshots of ICE analysis data with WT, HDR and INDEL frequencies for the corresponding samples from (FIG. 28c-28d) is shown in the right panel.

FIGS. 29a-29b: Gene targeting at the CCR5 locus in HSPCs using seemingly low activity gRNAs with AZD7648 treatment. FIG. 29a. HSPC were gene targeted at the CCR5 locus for knock-in of UBC-GFP-bGH-pA sequence using high activity (sg11) and low activity gRNAs (sg1 and 4). Allelic distribution of WT, INDEL and HDR frequencies following gene editing at the CCR5 locus with and without AZD7648 treatment (n=3). HDR frequency was determined by ddPCR analysis. WT and INDEL frequencies were determined by ICE analysis. Mean HDR to INDEL ratio is represented above the bars. All data are shown as mean±SEM. FIG. 29b. Representative screenshots of ICE analysis on PSCs gene targeted at the CCR5 locus (FIG. 29a) showing frequencies of WT and INDELs along with the pattern.

FIGS. 30a-30f: Gene targeting at the IL2RG locus in HSPC using seemingly low activity gRNAs with AZD7648 treatment. FIG. 30a. Schematic for gene targeting at the IL2RG locus for knock-in of codon-optimized cDNA and bGHpA in exon 1 using RNP/AAV6 gene editing with a high activity gRNA (sg1) and a low activity gRNA (sg6). FIG. 30b. Schematic for gene targeting at the IL2RG locus for knock-in of codon-optimized cDNA and bGHpA in exon 1 using RNP/AAV6 gene editing with a high activity gRNA (sg1) and two low activity gRNAs (sg5, 7). FIG. 30c. Allelic distribution of WT, INDEL and HDR frequencies in HSPCs gene targeted at the IL2RG locus with or without AZD7648 treatment (0.5 μM) using sg1 and 6 gRNAs (n=3) (FIG. 30a). HDR efficiency was measured through ddPCR analysis. WT and INDEL frequencies were measured by ICE analysis. Mean HDR to INDEL ratio is represented above the bars. All data are shown as mean±SEM. FIG. 30d. Allelic distribution of WT, INDEL and HDR frequencies in HSPCs gene targeted at the IL2RG locus with or without AZD7648 treatment (0.5 μM) using sg1, 5 and 7 (n=2) (FIG. 30b). HDR efficiency was measured through ddPCR analysis. WT and INDEL frequencies were measured by ICE analysis. Mean HDR to INDEL ratio is represented above the bars. All data are shown as mean±SEM. FIGS. 30e-30f. Representative screenshots of ICE analysis on HSPCs gene targeted at the IL2RG locus (FIG. 30c-30d) showing frequencies of WT and INDELs along with the pattern for sg1 and 6 (FIG. 30e) and sg5 and 7 (FIG. 30f).

FIGS. 31a-31c: AZD7648 improves the frequency of gene targeted cells with lower amounts of RNP in PSC and HSPC. FIG. 31a. Frequency of gene targeted cells measured by flow cytometry for GFP expression following gene editing at the CCR5 locus for knock-in of UBC-GFP-bGH-pA sequence with or without AZD7648 treatment (0.25 μM) using varying amounts of Cas9-RNP as indicated and fixed amount of AAV6 donor (MOI:2500) in PSCs (n=3). RNP-1× denotes 250 μg/ml of Cas9 protein complexed with 100 μg/ml of gRNA. RNP-0.5×, 0.2× and 0.1× denote 2-, 5-, and 10-fold lower concentrations of the RNP respectively. All data are shown as mean±SEM. FIG. 31b. Representative screenshots of ICE analysis on PSC gene targeted at CCR5 locus (FIG. 31a) showing frequencies of WT and INDELs along with the pattern. FIG. 31c. Representative screenshots of ICE analysis showing frequencies of WT, HDR and INDELs along with the pattern in HSPCs gene targeted at the STING1 locus for introducing V155M mutation with varying amounts of RNP as indicated.

FIGS. 32a-32b: AZD7648 improves the frequency of gene targeted cells with lower amounts of AAV6 in PSCs. FIG. 32a. Frequency of gene targeted cells measured by flow cytometry for GFP expression following gene editing at the CCR5 locus for knock-in of UBC-GFP-bGH-pA sequence in PSCs with or without AZD7648 treatment (0.25 μM) using a fixed amount of RNP and varying amounts of AAV6 donor (MOI. 100, 500, 1000, 2500, 5000 and 10000) (n=3). All data are shown as mean±SEM. FIG. 32b. Representative screenshots of ICE analysis on PSCs gene targeted at the CCR5 locus (FIG. 32a) showing frequencies of WT and INDELs along with the pattern.

FIGS. 33a-33b: AZD7648 improves the frequency of gene targeted cells with lower amounts of AAV6 in HSPCs. FIG. 33a. Frequency of gene targeted cells measured by flow cytometry for GFP expression following gene editing at the CCR5 locus for knock-in of UBC-GFP-bGH-pA sequence in HSPCs with or without AZD7648 treatment (0.25 μM) using a fixed amount of RNP and varying amounts of AAV6 donor (MOI. 100, 500, 1000, 2500, 5000 and 10000) (n=3). All data are shown as mean±SEM. FIG. 33b. Representative screenshots of ICE analysis on HSPC gene targeted at the CCR5 locus (FIG. 33a) showing frequencies of WT and INDELs along with the pattern.

FIGS. 34a-34d: AZD7648 improves gene targeting in T cells. FIG. 34a. Comparison of different DNA-PKcs inhibitors for gene targeting at the CCR5 locus for knock-in of UBC-GFP-bGHpA sequence at two different concentrations (2 and 0.5 μM) in T cells. Allelic gene targeting efficiency was measured by ddPCR analysis and cell viability was assessed by measuring live cell count and normalizing the number to the Mock sample (n=3). FIG. 34b. Frequency of gene targeted cells as measured by flow cytometry for GFP expression following UBC-GFP-bGH-pA sequence knock-in at the CCR5 locus in T cells with varying concentrations of AZD7648 (as indicated) using RNP/AAV6 gene editing (n=3). FIG. 34c. Percentage of viable cell count for the CCR5 gene targeted T cells (FIG. 34b) at 72 h post gene editing (n=3) relative to the mock cells. All data are shown as mean±SEM. FIG. 34d. Representative screenshots of ICE analysis showing frequencies of WT and INDELs along with the pattern in T cells gene targeted at the CCR5 locus with different concentrations of AZD7648 (FIG. 34b).

FIGS. 35a-35d: AZD7648 improves gene targeting in T cells with lower amounts of AAV6. FIG. 35a. Frequency of gene targeted cells as measured by flow cytometry for GFP following UBC-GFP-bGH-pA sequence knock-in at the CCR5 locus in T cells with or without AZD7648 (0.5 μM) treatment using a fixed amount of RNP and varying amounts of AAV6 donor (MOI. 1000, 2500, 5000 and 10000) (n=3). FIG. 35b. Percentage of viable cell count for the CCR5 gene targeted T cells (FIG. 35a) at 72 h post gene editing (n=3) relative to the mock cells. All data are shown as mean±SEM. FIG. 35c. Representative screenshots of ICE analysis showing frequencies of WT and INDELs along with the pattern in T cells gene targeted at the CCR5 locus with different amounts of AAV6 (FIG. 35a). FIG. 35d. T cells were gene targeted at the TRAC locus for knock-in of CD19 CAR with and without AZD treatment. Engineered CD19 CAR T cells were challenged with GFP+Nalm6 leukemia target cells in co-culture at an effector to target ratio of 1:1 for 72 hours. Potency of the CAR T cell cytotoxicity activity was monitored by the residual percentage of GFP+ target cells by flow cytometry at 24 h and 48 h post challenge. All data are shown as mean±SEM.

FIGS. 36a-36f: AZD7648 improves gene targeting in B cells and HBECs. FIG. 36a. Allelic distribution of WT, INDEL and HDR frequencies following gene editing at the CCR5 locus for knock-in of UBC-GFP-bGH-pA sequence in B cells with varying concentrations of AZD7648 (as indicated) using RNP/AAV6 gene editing (n=2). HDR frequency was measured by ddPCR. WT and INDEL frequencies were measured through ICE analysis. Mean HDR to INDEL ratio is represented above the bars. FIG. 36b. Representative screenshots of ICE analysis showing frequencies of WT and INDELs along with the pattern in B cells gene targeted at the CCR5 locus (FIG. 36a). FIG. 36c. Schematic for gene targeting at exon 1 of the CFTR gene for knock-in of SFFV-Citrine-pA sequence using RNP/AAV6 gene editing. FIG. 36d. Frequency of gene targeted cells as measured by flow cytometry for Citrine expression following CFTR-exon 1 gene editing (FIG. 36c) with or without AZD7648 treatment (0.5 μM) in HBECs (n=4). FIG. 36e. Fold change in the frequency of CFTR gene targeted HBECs (FIG. 36d) with AZD7648 (0.5 μM) treatment relative to the untreated cells. All data are shown as mean±SEM. FIG. 36f. CF patient derived HBECs were gene targeted at the CFTR locus for the correction of AF508 mutation in exon 11. Alignment of the CF and HDR sequences for the CFTR gene. AF508 mutation is shown in red for CF sequence and silent mutations are shown in green for HDR sequence. Left panel shows representative Sanger sequencing chromatograms for Mock, RNP+AAV6 (untreated (UNT) and AZD7648 treated (AZD-0.5 μM)) HBEC samples. Screenshots of ICE analysis data with WT, HDR and INDEL frequencies for corresponding samples is shown in the right panel.

### DETAILED DESCRIPTION

### 1. Introduction

As discussed above, there is a need for new and efficient methods for promoting HDR-mediated genomic editing in primary cells. In genomic loci with low levels of HDR, the DSB created by Cas9 RNP is predominantly repaired by NHEJ pathway, leading to the formation of indels¹⁸. Thus, recent studies have explored the possibility of inhibition of NHEJ repair as a way to improve the efficiency of HDR-based gene targeting¹⁹. The present disclosure provides methods for improving the efficiency of homology directed repair (HDR)-mediated modification of genomic sequences in primary cells, and is based in part on the discovery by the inventors that DNA-PK inhibitors are able to promote HDR-mediated genome editing. The methods provided herein involve the introduction into cells of single guide RNAs (sgRNAs), RNA-guided nucleases (e.g., Cas9), homologous repair templates, and DNA-PK inhibitors. The methods can be used, e.g., to integrate cDNAs encoding functional proteins into cells to correct or compensate for mutations in cells from a subject with a genetic disorder, or to modify endogenous genomic sequences for any purpose using HDR. As demonstrated in the Examples herein and described throughout the present disclosure, the provided methods enhance gene targeting efficiency in hPSCs, HSPCs, T cells, B cells, and human bronchial epithelial cells (HBECs). Also provided herein are methods for treating various genetic diseases and cancer using ex vivo gene edited cell-based therapeutics.

DNA-dependent protein kinase (DNA-PK) is a key protein in NHEJ repair pathway that is involved in processing of the broken ends of the DSB. Small molecule inhibitors against DNA-PK have been developed, as it is considered as a potential target for anti-tumor therapeutic²⁰. Some of these compounds have been tested for enhancing gene targeting efficiencies¹⁹. Recent studies have shown that DNA-PK inhibitor, M3814 can enhance the gene targeting efficiency in human PSCs and T cells^{18,21}. But, a recent study has shown that AZD7648 and VX984 are two potent DNA-PK inhibitors with a higher specificity than M3814^{22,23}. All these three small molecules are currently being tested in clinical trials for the treatment of solid tumors²⁴. As described in the Examples herein, the effects of AZD7648, M3814, VX984 and few other DNA-PK inhibitors treatment on HDR-based gene targeting using the Cas9 RNP and AAV6 based gene editing platform were compared. It was found that AZD7648 is more potent than M3814 and VX984 in enhancing the gene targeting efficiency in various human primary stem cells. In some embodiments, AZD7648 treatment can promote gene targeting frequency for small nucleotide changes as high as 100%, and large sequence integrations can be achieved at up to 80% frequency. In some embodiments, AZD7648 treatment can improve gene targeting across different genomic loci in hematopoietic stem and progenitor cells (HSPCs) and pluripotent stem cells (PSCs). In some embodiments, AZD7648 treatment can improve gene targeting with seemingly inactive and low activity gRNAs. In some embodiments, AZD7648 treatment can reduce the amounts of RNP and AAV6 with maintenance of high gene targeting efficiencies. In some embodiments, AZD7648 treatment can improve gene targeting in primary human T and B cells without affecting immune cell function. In particular embodiments, the sgRNA and nuclease are delivered to cells as ribonucleoprotein (RNP) complexes (e.g., by electroporation), and the DNA-PK inhibitor is delivered (e.g., by addition of the DNA-PK inhibitor to cell growth medium) before, concurrently with, or after delivery of the RNP complexes, followed by the transduction of the homologous repair template using an AAV6 viral vector. The introduction of the DNA-PK inhibitor transiently increases the rate of HDR and reduces non-homologous end-joining (NHEJ) in the primary cells, and also permits the use of lower amounts of donor template (e.g., reduced MOIs when using viral vectors such as AAV6) than is possible in the absence of DNA-PK inhibitor, while still achieving high levels of HDR in the cells and high levels of engraftment in vivo. This system can be used to modify any human cell, including hPSCs, HSPCs, T cells, B cells, and HBECs. In particular embodiments, CD34⁺ HSPCs are used.

### 2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8×, 0.81×, 0.82×, 0.83×, 0.84×, 0.85×, 0.86×, 0.87×, 0.88×, 0.89×, 0.9×, 0.91×, 0.92×, 0.93×, 0.94×, 0.95×, 0.96×, 0.97×, 0.98×, 0.99×, 1.01×, 1.02×, 1.03×, 1.04×, 1.05×, 1.06×, 1.07×, 1.08×, 1.09×, 1.1×, 1.11×, 1.12×, 1.13×, 1.14×, 1.15×, 1.16×, 1.17×, 1.18×, 1.19×, and 1.2×. Thus, “about X” is intended to teach and provide written description support for a claim limitation of, e.g., “0.98×.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

“DNA-dependent protein kinase” (“DNA-PK”) is a nuclear serine/threonine protein kinase complex composed of the catalytic subunit (DNA-PKcs) and a heterodimer of Ku proteins (Ku70/Ku80). DNA-PK is a member of the phosphatidylinositol 3-kinase-related kinase (PIKK) family of protein kinases and plays a role in DNA double strand break (DSB) repair, serving to maintain genomic integrity, and in the process of V(D)J recombination.

A “DNA-PK” inhibitor is an agent that inhibits a function of DNA-PK. A DNA-PK inhibitor of the present disclosure may selectively inhibit the kinase DNA-PK, or may non-selectively inhibit DNA-PK and also inhibit other kinases. Examples of DNA-PK inhibitors are discussed in detail below.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of an introduced cDNA or encoded protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “homologous repair template” or “homologous donor template” refers to a polynucleotide sequence that can be used to repair a double stranded break (DSB) in the DNA, e.g., a CRISPR/Cas9-mediated break at a locus targeted by a herein-described sgRNA as induced using the herein-described methods and compositions. The homologous repair template comprises homology to the genomic sequence surrounding the DSB, i.e., comprising target locus homology arms as described herein. In some embodiments, two distinct homologous regions are present on the template, with each region comprising at least 50, 100, 200, 300, 400, 500, 600, 700, 800, 900 or more nucleotides or more of homology with the corresponding genomic sequence. In particular embodiments, the templates comprise two homology arms comprising about 500 nucleotides of homology extending from either site of the sgRNA target site. The repair template can be present in any form, e.g., on a plasmid that is introduced into the cell, as a free-floating doubled stranded DNA template (e.g., a template that is liberated from a plasmid in the cell), or as single-stranded DNA. In particular embodiments, the template is present within a viral vector, e.g., an adeno-associated viral vector such as AAV6. In some embodiments, the templates of the disclosure a codon-optimized, e.g., full-length, codon-optimized cDNAs, as well as, typically, a polyadenylation signal such as from bovine growth hormone or rabbit beta-globin. In some embodiments, the cDNA comprises a promoter, operably linked to the cDNA. In some embodiments, the template comprises a sequence other than a cDNA, e.g., a sequence designed to correct a specific mutation in a genomic locus, or to introduce a specific deletion or insertion into a locus. The process of repairing a double-stranded break using a homologous donor template is referred to as Homology Directed Repair (HDR).

As used herein, “homologous recombination” or “HR” refers to insertion of a nucleotide sequence during repair of double-strand breaks in DNA via homology-directed repair (HDR) mechanisms. This process uses a “donor template” or “homologous repair template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems. In particular embodiments, HR involves double-stranded breaks induced by CRISPR-Cas9.

### 3. Methods of Enhancing HDR

The present disclosure provides methods for improving the efficiency of genomic editing through homology-directed repair (HDR), e.g., for editing genomic sequences or integrating cDNAs into endogenous loci in cells, through the administration of a DNA-PK inhibitor to the cells. The present methods and compositions allow genomic editing to be performed with higher rates of HDR and with lower rates of non-homologous end-joining (NHEJ) and, as a result, of insertions and deletions (indels). Further, the methods allow for high levels of HDR and cell engraftment to be achieved with lower levels of administered donor templates, e.g., using lower multiplicities of infection (MOI) when donor templates are introduced using viral vectors such as adeno-associated viral vectors (AAV) such as AAV6. The effects observed using DNA-PK inhibitors in cells is transient, allowing HDR to be achieved without introducing longer-term genomic instability as might be observed, e.g. using nucleic acids encoding other NHEJ inhibitors.

In particular embodiments, the cells are primary human cells, including stem cells such as CD34⁺ hematopoietic stem and progenitor cells (HSPCs) or hematopoietic stem cells (HSCs). In some embodiments, cells from a subject are modified using the methods described herein and then reintroduced into the subject. For example, the cells can be taken from a subject with a genetic condition and the methods used to integrate a functional cDNA into the genome of the cells, wherein the expression of the cDNA in the modified cells in vivo restores protein activity that is missing or deficient in the subject or is otherwise beneficial to the subject.

The present disclosure is based in part on the identification that DNA-PK inhibitors, e.g., AZD7648, can effectively and safely increase HDR, decrease NHEJ, and decrease indels, when introduced together with a guide RNA and RNA-guided nuclease such as Cas9, and with a homologous donor template. In particular embodiments, the guide RNA and RNA-guided nuclease are introduced as a ribonucleoprotein (RNP), for example by electroporation. In particular embodiments, the DNA-PK inhibitor is introduced before, concurrently with, or after introduction of the RNP.

### 4. DNA-PK Inhibitors

Various DNA-PK inhibitors may be used in the practice of the methods provided herein. In some embodiments, the DNA-PK inhibitor is a compound represented by the following formula (I):

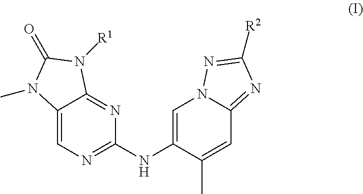

wherein:
R¹ is a cyclohexyl, tetrahydrofuranyl or oxanyl ring, each of which is optionally substituted by one or more groups selected from hydroxyl, methoxy, and methyl; and
R² is hydrogen or methyl,
   or a pharmaceutically acceptable salt thereof.

In some embodiments, the DNA-PK inhibitor is a compound represented by formula (I), wherein R¹ is oxanyl. In some embodiments, the DNA-PK inhibitor is a compound represented by formula (I), wherein R¹ is oxan-4-yl. In some embodiments, the DNA-PK inhibitor is a compound represented by formula (I), wherein R² is hydrogen.

The term “cyclohexyl ring” refers to a carbocylic ring containing six carbon atoms and no heteroatoms. The term “tetrahydrofuranyl ring” includes tetrahydrofuran-3-yl, the structure of which is shown below:

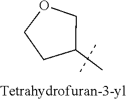

The term “oxanyl ring” includes oxan-3-yl and oxan-4-yl groups, the structures of which are shown below:

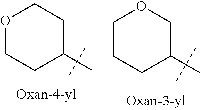

In the above structures, the dashed line indicates the bonding position of the relevant group.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

The term “substituted” means that one or more hydrogens (for example 1 or 2 hydrogens, or alternatively 1 hydrogen) on the designated group is replaced by the indicated substituent (s) (for example 1 or 2 substituents, or alternatively 1 substituent), provided that any atom(s) bearing a substituent maintains a permitted valency. Substituent combinations encompass only stable compounds and stable synthetic intermediates.

The term “heteroatom” refers to an atom other than a carbon which may be present in a carbon backbone of a linear, branched, or cyclic compound. Heteroatoms include, but are not limited to, oxygen (O), nitrogen (N), sulfur (S), phosphorus (P) and silicon (Si). Heteroatoms can be present in their reduced forms, e.g., as —OH, —NH, or —SH, or in their oxidized forms, e.g., as —S(O)— and —S(O)₂—.

“Stable” means that the relevant compound or intermediate is sufficiently robust to be isolated and have utility either as a synthetic intermediate or as an agent having potential therapeutic utility. If a group is not described as “substituted”, or “optionally substituted”, it is to be regarded as unsubstituted (i.e. that none of the hydrogens on the designated group have been replaced).

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zlirich:Wiley-VCH/VHCA, 2002.

A suitable pharmaceutically acceptable salt of a compound of formula (I) is, for example, an acid-addition salt. An acid addition salt of a compound of formula (I) may be formed by bringing the compound into contact with a suitable inorganic or organic acid under conditions known to the skilled person. An acid addition salt may for example be formed using an inorganic acid selected from the group consisting of hydrochloric acid, hydrobromic acid, sulphuric acid and phosphoric acid. An acid addition salt may also be formed using an organic acid selected from the group consisting of trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid.

Compounds and salts described in this specification may exist in solvated forms and unsolvated forms. For example, a solvated form may be a hydrated form, such as a hemi-hydrate, a mono-hydrate, a di-hydrate, a tri-hydrate or an alternative quantity thereof. The disclosure encompasses all such solvated and unsolvated forms of compounds of formula (I), particularly to the extent that such forms possess DNA-PK inhibitory activity.

Atoms of the compounds and salts described in this specification may exist as their isotopes. The disclosure encompasses all compounds of formula (I) where an atom is replaced by one or more of its isotopes (for example a compound of formula (I) where one or more carbon atom is an ₁₁C or ₁₃C carbon isotope, or where one or more hydrogen atoms is a 2H or 3H isotope, or where one or more nitrogen atoms is a ₁₅N isotope or where one of more oxygen atoms is an ₁₇O or ₁₈O isotope).

The DNA-PK compounds of the disclosure, or their pharmaceutically acceptable salts may contain one or more asymmetric centers and may thus give rise to enantiomers, diastereomers, geometric isomers, individual isomers and other stereoisomeric forms that may be defined, in terms of absolute stereochemistry, as (R)- or (S)- or, as (D)- or (L)- for amino acids. The present disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

Some of the compounds of formula (I) may be crystalline and may have more than one crystalline form. It is to be understood that the disclosure encompasses any crystalline or amorphous form, or mixtures thereof, which possess properties useful in DNA-PK inhibitory activity. It is well known how to determine the efficacy of a crystalline or amorphous form by standard tests.

It is generally known that crystalline materials may be analysed using conventional techniques such as, for example, X-Ray Powder Diffraction (hereinafter XRPD) analysis and Differential Scanning Calorimetry (DSC).

In some embodiments, the DNA-PK inhibitor is the compound AZD7648, 7-methyl-2-((7-methyl-[1,2,4]triazolo[l,5-a]pyridin-6-yl)amino)-9-(tetrahydro-2H-pyran-4-yl)-7,9-dihydro-8H-purin-8-one represented by the following formula:

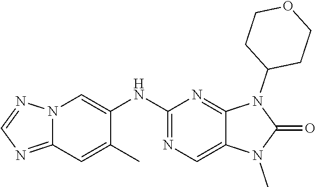 [SMILES: Cc1cc2ncnn2cc1Nc1ncc2c(n1)n(C1CCOCC1)c(=O)n2C]

or a pharmaceutically acceptable salt thereof.

In some embodiments, the DNA-PK inhibitor is the compound VX984 represented by the following formula:

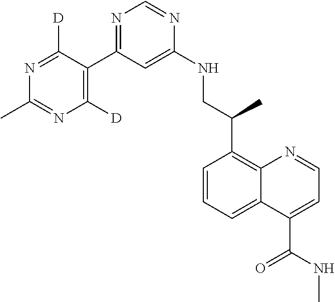

or a pharmaceutically acceptable salt thereof.

In some embodiments, the DNA-PK inhibitor is the compound BAY8400 represented by the following formula:

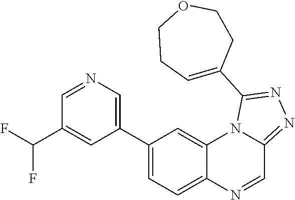 [SMILES: FC(F)c1cncc(-c2ccc3ncc4nnc(C5=CCCOCC5)n4c3c2)c1]

or a pharmaceutically acceptable salt thereof.

In addition to those described above, other DNA-PK inhibitors may be used in the methods provided herein. In some embodiments, a DNA-PK inhibitor useful in the methods provided herein has very high specificity for the catalytic subunit of DNA-PK (DNA-PKcs). In some embodiments, the DNA-PK inhibitor binds strongly to DNA-PKcs and does not bind (or binds weakly) to other PIKK family kinases (e.g., ATM, ATR, PI3Kα, PI3Kβ, PI3Kγ, PI3Kδ, and/or mTOR). In some embodiments, the DNA-PK inhibitor has an IC50 in the range of about 20 nM to about 1 μM (e.g., about 25 nM to about 1 μM, about 30 nM to about 1 μM, about 35 nM to about 1 μM, about 40 nM to about 1 μM, about 45 nM to about 1 μM, about 50 nM to about 1 μM, about 55 nM to about 1 μM, about 60 nM to about 1 μM, about 65 nM to about 1 μM, or about 70 nM to about 1 μM) for DNA-PKcs and an IC50 of greater than 1 μM for other PIKK family kinases (e.g., ATM, ATR, PI3Kα, PI3Kβ, PI3Kγ, PI3Kδ, and/or mTOR). Methods for evaluating the binding strength and/or specificity of an enzyme (e.g., a DNA-PK inhibitor) are known in the art and are demonstrated, e.g., in Example 1 herein.

The DNA-PK inhibitors used in the methods provided herein are either available from commercial suppliers or are prepared by methods known to those skilled in the art following procedures set forth in references such as Fieser and Fieser's Reagents for Organic Synthesis, Vol. 1-28 (Wiley, 2016); March's Advanced Organic Chemistry, 7^{th} Ed. (Wiley, 2013); and Larock's Comprehensive Organic Transformations, 2^{nd} Ed. (Wiley, 1999). The DNA-PK inhibitors can be isolated and purified if desired using conventional techniques including, but not limited to, filtration, distillation, crystallization, chromatography, and the like. Such materials can be characterized using conventional means, including measuring physical constants and obtaining spectral data.

### 5. Introduction of DNA-PK Inhibitor into Cells

The DNA-PK inhibitor can be introduced into cells in any of a number of ways, e.g., by addition of the DNA-PK inhibitor to cell growth medium (e.g., when cells are plated after electroporation). In some embodiments, the DNA-PK inhibitor is introduced together with RNPs comprising an sgRNA and RNA-guided nuclease. In some embodiments, the DNA-PK inhibitor is introduced before introduction of RNPs comprising an sgRNA and RNA-guided nuclease. In some embodiments, the DNA-PK inhibitor is introduced after introduction of RNPs comprising an sgRNA and RNA-guided nuclease. In some embodiments, the DNA-PK inhibitor is introduced concurrently with introduction of RNPs comprising an sgRNA and RNA-guided nuclease. Methods for introducing small molecules (e.g., a DNA-PK inhibitor) into cells are known in the art (see, e.g., Yang and Hinner, 2015, Methods Mol. Biol. 1266:29-53).

In some embodiments, the DNA-PK inhibitor is a druglike small molecule. In some embodiments, a particular DNA-PK inhibitor may be selected for and/or modified for druglikeness. The term “druglike small molecule” as used herein generally refers to a low molecular weight (e.g., less than 900 daltons) organic compound, either naturally occurring or synthetic, that may regulate a biological process (e.g., when administered as a drug). Because of their low molecular weight, druglike small molecules are generally able to rapidly diffuse across cell membranes and often possess oral bioavailability (i.e., then can be absorbed into the body through intestinal epithelial cells). Additional characteristics may be assessed to evaluate the druglikeness of a small molecule, as discussed further below.

In some embodiments, evaluation of druglikeness may involve assessment of the small molecule for compliance with the rule of five (also known as Lipinski's rule of five), which describes molecular properties important for a drug's pharmacokinetics (e.g., absorption, distribution, metabolism, and excretion) in the human body (See, e.g., Lipinski et al. 2001. Adv. Drug Deliv. Rev. 46(1-3):3-26). In general, the rule of five includes the following characteristics: 1) no more than 5 hydrogen bond donors (i.e., the total number of nitrogen-hydrogen and oxygen-hydrogen bonds); 2) no more than 10 hydrogen bond acceptors (i.e., all nitrogen or oxygen atoms); 3) a molecular mass less than 500 daltons; and 4) an octanol-water partition coefficient (see, e.g., Leo et al. 1971. Chem Rev. 71(6):525-616) that does not exceed 5. As such, in some embodiments, the DNA-PK inhibitors used in the methods provided herein are characterized by one or more properties selected from the group consisting of: 1) a total number of hydrogen bond donating groups equal to or less than 5; 2) a total number of hydrogen bond accepting groups equal to or less than 10; 3) a molecular mass less than 500 daltons; and 4) an octanol-water partition coefficient equal to or less than 5.

The DNA-PK inhibitor can be introduced into cells at any suitable concentration, i.e., a concentration sufficient to increase HDR in the cell and decrease NHEJ, indels, etc. The precise concentration used will depend upon the cell type, the targeted locus, the nature of genetic modification desired, and other factors known to one of skill in the art. The effect of DNA-PK inhibitor is concentration dependent, and HDR in HSPCs, for example, increases in a dose dependent manner. In some embodiments, the DNA-PK inhibitor is present at a concentration of from 2 ng/ml (0.005 μM) to 2000 ng/ml (5 μM), e.g., about 5 ng/ml to about 1800 ng/ml, about 20 ng/ml to about 1700 ng/ml, about 50 ng/ml to about 1600 ng/ml, about 100 ng/ml to about 1500 ng/ml, about 150 ng/ml to about 1400 ng/ml, or about 175 ng/ml to about 1200 ng/ml. In some embodiments, the DNA-PK inhibitor is present at a concentration of about 10 ng/ml, about 20 ng/ml about 40 ng/ml, about 60 ng/ml, about 80 ng/ml, about 100 ng/ml, about 120 ng/ml, about 140 ng/ml, about 160 ng/ml, about 180 ng/ml, about 190 ng/ml, about 200 ng/ml, about 220 ng/ml, about 240 ng/ml, about 250 ng/ml, or more. In particular embodiments, the DNA-PK inhibitor is introduced at about 190 ng/ml (0.5 μM).

In particular embodiments, the DNA-PK inhibitor introduced into cells is transient. For example, in some embodiments, there is a reduction of at least about 50%, 60%, 70%, 80%, 90%, 95%, 96%, 97%, 98%, 99% or more of the DNA-PK inhibitor in the cells as detected, e.g., by mass spectroscopy, within 4 hours after introduction of the DNA-PK inhibitor into the cells, relative to the amount present immediately after introduction.

### 6. Other Components

sgRNAs

The DNA-PK inhibitors as described herein are introduced into cells in conjunction with single guide RNAs (sgRNAs). sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence, and a constant region that mediates binding to Cas9 or another RNA-guided nuclease. The sgRNA can target any sequence within the target gene adjacent to a PAM sequence. The sgRNAs used in the present methods and compositions can target any locus that is to be modified or edited. In some embodiments, the target gene or locus is a safe harbor locus such as CCR5 or a locus associated with a genetic disorder, such as sickle cell disease, β-thalassemia, X-linked severe combined immunodeficiency (e.g., SCID-X1), X-linked chronic granulomatous disease (X-CGD), cystic fibrosis, lysosomal storage disorders such as mucopolysaccharidosis type 1, Gaucher's disease, or Krabbe disease, and others, and the methods are used to correct a mutated copy of the gene in a patient. A non-limiting list of genes that can be targeted or introduced using the present methods includes HBB, CYBB, CCR5, IL2RG, HBA1, HBA2, CFTR, STING1, and others.

In some embodiments of the methods provided herein, sgRNAs targeting one locus are introduced into cells. In some embodiments, sgRNAs targeting more than one locus (e.g., 2 loci, 3 loci, 4 loci, or more) are introduced into cells. Introduction of sgRNAs targeting more than one locus may promote HDR-mediated genome editing at more than one locus (i.e., multiplexing).

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgRNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g, 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the tracrRNA provides a binding scaffold for the Cas nuclease.

In some embodiments, e.g., when the methods are used to introduce a functional full-length cDNA to the genome, the target sequence is located near the translational start site of the gene, such that the full-length cDNA can be expressed under the control of the endogenous promoter. In other embodiments, the target sequence can be elsewhere in a gene or locus, e.g., to modify the sequence at the site of a mutation, to introduce a regulatory element, to introduce a deletion to remove protein function, to introduce an expression cassette comprising a coding sequence operably linked to a promoter, etc. It will be understood that the present methods can be used to enhance the rate of HDR for any purpose, and using sgRNAs targeting any part of a gene or genome.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothiate internucleotide linkages, 2′-O-methyl-3′-phosphoacetate modifications, 2′-fluoro-pyrimidines, S-constrained ethyl sugar modifications, or others, at one or more nucleotides. In particular embodiments, the sgRNAs comprise 2′-O-methyl-3′-phosphorothioate (MS) modifications at one or more nucleotides (see, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the 5′ and 3′ ends of the sgRNA.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### RNA-Guided Nucleases

The sgRNAs are used together with an RNA-guided nuclease, e.g. a CRISPR-Cas nuclease. Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA of the present disclosure and being guided to and cleaving the specific sequence targeted by the targeting sequence of the sgRNA. In particular embodiments, the Cas9 is from Streptococcus pyogenes. In some embodiments, a high fidelity Cas9 nuclease is used.

Also disclosed herein are CRISPR/Cas or CRISPR/Cpf1 systems that target and cleave DNA at a locus of interest. An exemplary CRISPR/Cas system comprises (a) a Cas (e.g., Cas9) or Cpf1 polypeptide or a nucleic acid encoding said polypeptide, (b) an sgRNA that hybridizes specifically to the locus of interest, or a nucleic acid encoding said guide RNA, (c) a donor template as described herein, and (d) a DNA-PK inhibitor. In particular embodiments, the CRISPR/Cas system comprises an RNP comprising an sgRNA targeting the locus of interest and a Cas protein such as Cas9.

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the locus of interest to carry out the methods disclosed herein.

Introducing the sgRNA and RNA-Guided Nuclease into Cells

The sgRNA and nuclease can be introduced into a cell using any suitable method, e.g., by introducing one or more polynucleotides encoding the sgRNA and the nuclease into the cell, e.g., using a vector such as a viral vector or delivered as naked DNA or RNA, such that the sgRNA and nuclease are expressed in the cell. In some embodiments, one or more polynucleotides encoding the sgRNA, the nuclease or a combination thereof are included in an expression cassette. In some embodiments, the sgRNA, the nuclease, or both sgRNA and nuclease are expressed in the cell from an expression cassette. In some embodiments, the sgRNA, the nuclease, or both sgRNA and nuclease are expressed in the cell under the control of a heterologous promoter. In some embodiments, one or more polynucleotides encoding the sgRNA and the nuclease are operatively linked to a heterologous promoter. In particular embodiments, the sgRNA and nuclease are assembled into ribonucleoproteins (RNPs) prior to delivery to the cells. The RNPs can be introduced into the cell using any suitable method, e.g., microinjection, electroporation, or other chemical transfection (e.g., lipid vesicles, osmocytosis, soluporation or other permeabilization techniques, etc.) or physical transfection methods (e.g., mechanical transfection, membrane disruption or permeabilization, etc.). In particular embodiments, the RNPs are introduced into the cell by electroporation.

In some embodiments, the sgRNA is introduced into cells at a concentration of about 15 μg/ml to about 300 μg/ml, e.g., 15 μg/ml, 20 μg/ml, 30 μg/ml, 40 μg/ml, 50 μg/ml, 60 μg/ml, 70 μg/ml, 75 μg/ml, 80 μg/ml, 85 μg/ml, 90 μg/ml, 95 μg/ml, 100 μg/ml, 105 μg/ml, 110 μg/ml, 115 μg/ml, 120 μg/ml, 125 μg/ml, 130 μg/ml, 135 μg/ml, 140 μg/ml, 145 μg/ml, 150 μg/ml, 175 μg/ml, 200 μg/ml, 225 μg/ml, 250 μg/ml, 275 μg/ml, or 300 μg/ml. In some embodiments, the sgRNA is introduced into cells at a concentration of less than about 150 μg/ml (e.g., 150 μg/ml, 100 μg/ml, 50 μg/ml, 30 μg/ml, or 15 μg/ml) in the presence of DNA-PK inhibitor. In some embodiments (e.g., as demonstrated in Example 4 herein), the sgRNA is introduced into cells in the presence of DNA-PK inhibitor at a concentration that is 1-fold, 2-fold, 5-fold, 10-fold or more lower than a standard or recommended concentration in the absence of the DNA-PK inhibitor.

In some embodiments, the nuclease is introduced into cells at a concentration of about 30 μg/ml to about 400 μg/ml, e.g., 30 μg/ml, 40 μg/ml, 50 μg/ml, 60 μg/ml, 70 μg/ml, 80 μg/ml, 90 μg/ml, 100 μg/ml, 110 μg/ml, 120 μg/ml, 125 μg/ml, 130 μg/ml, 135 μg/ml, 140 μg/ml, 145 μg/ml, 150 μg/ml, 155 μg/ml, 160 μg/ml, 165 μg/ml, 170 μg/ml, 175 μg/ml, 200 μg/ml, 225 μg/ml, 250 μg/ml, 275 μg/ml, 300 μg/ml, 325 μg/ml, 350 μg/ml, 375 μg/ml, or 400 μg/ml. In some embodiments, the nuclease is introduced into cells at a concentration of less than about 300 μg/ml (e.g., 250 μg/ml, 200 μg/ml, 150 μg/ml, 60 μg/ml, or 30 μg/ml) in the presence of DNA-PK inhibitor. In some embodiments (e.g., as demonstrated in Example 4 herein), the nuclease is introduced into cells in the presence of DNA-PK inhibitor at a concentration that is 1-fold, 2-fold, 5-fold, 10-fold or more lower than a standard or recommended concentration in the absence of the DNA-PK inhibitor.

Techniques for insertion of transgenes, including large transgenes, capable of expressing functional proteins, including enzymes, cytokines, antibodies, and cell surface receptors are known in the art (See, e.g. Bak and Porteus, Cell Rep. 2017 Jul. 18; 20(3): 750-756 (integration of EGFR); Kanojia et al., Stem Cells. 2015 October; 33(10):2985-94 (expression of anti-Her2 antibody); Eyquem et al., Nature. 2017 Mar. 2; 543(7643):113-117 (site-specific integration of a CAR); O'Connell et al., 2010 PLoS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30; 388(10043):476-87 (expression of arylsulfatase A in ex vivo gene therapy to treat MLD); Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is herein incorporated by reference in its entirety.

### Homologous Repair Templates

The homologous repair template used in the present methods can be any template used for genomic editing purposes, e.g., to integrate a cDNA or other sequence into a corresponding endogenous locus or a safe harbor locus, to introduce a deletion, insertion, or sequence modification into a targeted genomic locus, or for any other method wherein a genomic locus is cleaved using an sgRNA and RNA-guided nuclease such as Cas9, and the cleaved sequence is modified via HDR using a homologous donor template.

In some embodiments, the methods are used to introduce a cDNA into a targeted genomic locus. For example, in some embodiments, the methods can be used to integrate a cDNA such as a functional, codon-optimized cDNA into the genome of cells of a subject with a genetic disorder caused by a deficit or absence in the protein encoded by the cDNA, or a genetic or other disorder that can be treated or ameliorated in any way by the expression of the cDNA.

In some embodiments, the cDNA is integrated, e.g., at the translational start site of the endogenous locus, such that the cDNA is expressed under the control of the endogenous promoter and other regulatory elements. In other embodiments, the template comprises a promoter, operably linked to the cDNA, e.g., when the cDNA is integrated in a safe harbor locus such as the C—C chemokine receptor type 5 (CCR5) locus. In such embodiments, any promoter that can induce expression of the therapeutic protein in the modified cells can be used, including endogenous and heterologous promoters, inducible promoters, constitutive promoters, cell-specific promoters, and others. In some embodiments, the promoter is the phosphoglycerate kinase (PGK) promoter, the spleen focus-forming virus (SFFV) promoter, or the CD68 promoter.

In some instances, in addition to the promoter, the transgene is optionally linked to one or more regulatory elements such as enhancers or post-transcriptional regulatory sequences. For example, one can include regulatory sequences (microRNA (miRNA) target sites) in the RNA to avoid expression in certain tissues (post-transcriptional targeting). In some instances, the expression control sequence functions to express the therapeutic transgene following the same expression pattern as in normal individuals (physiological expression) (See Toscano et al., Gene Therapy (2011) 18, 117-127 (2011), incorporated herein by reference in its entirety for its references to promoters and regulatory sequences).

In some embodiments, the cDNA in the homologous repair template is codon-optimized, e.g., comprises at least 70%, 75%, 80%, 85%, 90%, 95%, or more homology to the wild-type cDNA sequence, or to a fragment thereof.

In particular embodiments, the template further comprises a polyA sequence or signal, e.g., a bovine growth hormone polyA sequence or a rabbit beta-globin polyA sequence, at the 3′ end of the cDNA. In particular embodiments, a Woodchuck Hepatitis Virus Posttranscriptional Regulatory Element (WPRE) is included within the 3′UTR of the template, e.g., between the 3′ end of the cDNA coding sequence and the 5′ end of the polyA sequence, so as to increase the expression of the cDNA. Any suitable WPRE sequence can be used; See, e.g., Zufferey et al. (1999) J. Virol. 73(4):2886-2892; Donello, et al. (1998). J Virol 72: 5085-5092; Loeb, et al. (1999). Hum Gene Ther 10: 2295-2305; the entire disclosures of which are herein incorporated by reference).

In particular embodiments, the cDNA (or cDNA and polyA signal) is flanked in the template by homology regions corresponding to the targeted locus. For example, an exemplary template can comprise, in linear order: a first genomic homology region, an optional promoter, a cDNA, a polyA sequence, and a second genomic homology region, where the first and second homology regions are homologous to the genomic sequences extending in either direction from the sgRNA target site. The homology regions can be of any size, e.g., 100-1000 bp, 300-800 bp, 400-600 bp, or about 100, 200, 300, 400, 500, 600, 700, 800, 900, 1000, or more base pairs.

In some embodiments of the methods provided herein, one homologous repair template is introduced into cells. In some embodiments, more than one (e.g., 2, 3, 4, or more) different homologous repair templates are introduced into cells. Different homologous repair templates may comprise, e.g., different cDNA sequences, different homology arms, or any other different sequences (e.g., different sequences for any of the elements described above). Introduction of more than one homologous repair template may promote HDR-mediated genome editing at more than one locus (i.e., multiplexing).

Introduction of Donor Templates into Cells

Any suitable method can be used to introduce the polynucleotide, or donor construct, into the primary cells. In particular embodiments, the polynucleotide is introduced using a recombinant adeno-associated viral vector, e.g., rAAV6. In some instances, the donor template is single stranded, double stranded, a plasmid or a DNA fragment. In some instances, plasmids comprise elements necessary for replication, including a promoter and optionally a 3′ UTR.

Further disclosed herein are vectors comprising (a) one or more nucleotide sequences homologous to the locus of interest, and (b) a cDNA as described herein. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises: (1) a viral vector backbone, e.g. an AAV backbone, to generate virus; (2) arms of homology to the target site of at least 200 bp but ideally at least 400 bp on each side to assure high levels of reproducible targeting to the site (see, Porteus, Annual Review of Pharmacology and Toxicology, Vol. 56:163-190 (2016); which is hereby incorporated by reference in its entirety); (3) a cDNA encoding a functional protein and capable of expressing the functional protein, optionally a promoter, a polyA sequence, and optionally a WPRE element; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb, 3-4 kb, 4-5 kb, 5-6 kb, 6-7 kb, 7-8 kb, or larger.

In some embodiments, viral vectors, e.g., AAV6 vector, is transduced at a multiplicity of infection (MOI) of, e.g., about 1×10³, 5×10³, 1×10⁴, 5×10⁴, 1×10⁵, between 2×10⁴ and 1×10⁵ viruses per cell, or less than 1×10⁵. In particular embodiments, the viral vector is introduced at an MOI of less than about 2500, e.g., about 2000, 1900, 1800, 1700, 1600, 1500, 1400, 1300, 1200, 1100, 1000, 900, 850, 800, 750, 700, 675, 650, 625, 600, 550, 500, 450, 400, or less. In particular embodiments, the viral vector is introduced at an MOI of about 500 in the presence of the DNA-PK inhibitor. In some embodiments, the viral vector is administered in the presence of the DNA-PK inhibitor at an MOI that is 1-fold, 2-fold, 3-fold, 4-fold, or more lower than a standard or recommended MOI in the absence of the DNA-PK inhibitor.

Suitable marker genes are known in the art and include Myc, HA, FLAG, GFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (tNGFR), operably linked to a promoter such as the Ubiquitin C promoter.

The inserted construct can also include other safety switches, such as a standard suicide gene into the locus (e.g. iCasp9) in circumstances where rapid removal of cells might be required due to acute toxicity. The present disclosure provides a robust safety switch so that any engineered cell transplanted into a body can be eliminated, e.g., by removal of an auxotrophic factor. This is especially important if the engineered cell has transformed into a cancerous cell.

The present methods allow for the efficient integration of the donor template at the endogenous locus of interest. In some embodiments, the present methods allow for the insertion of the donor template in 20%, 25%, 30%, 35%, 40%, or more cells, e.g., cells from an individual with a condition to be treated using the present methods and/or compositions. The methods also allow for high levels of expression of protein in cells, e.g., cells from an individual with an integrated cDNA as described herein, e.g., levels of expression that are at least about 70%, 75%, 80%, 85%, 90%, 95%, or more relative to the expression in healthy control cells.

### Cells

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys. In particular embodiments, the cells are human cells, e.g., human cells from a subject with a genetic disorder or condition.

In particular embodiments, the cells used in the present methods are primary cells, i.e., cells taken directly from a living tissue (e.g., biopsy, blood sample, etc.). In some embodiments, the cell is an embryonic stem cell, a stem cell, a progenitor cell, a pluripotent stem cell, an induced pluripotent stem cell (iPSC), a somatic stem cell, a differentiated cell, a mesenchymal stem cell or a mesenchymal stromal cell, an airway basal stem cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). In particular embodiments, the cells are CD34+ hematopoietic stem and progenitor cells (HSPCs), e.g., cord blood-derived (CB), adult peripheral blood-derived (PB), or bone marrow derived HSPCs. HSPCs can be isolated from a subject, e.g., by collecting mobilized peripheral blood and then enriching the HSPCs using the CD34 marker.

To avoid immune rejection of the modified cells when administered to a subject, the cells to be modified are preferably derived from the subject's own cells. Thus, preferably the mammalian cells are autologous cells from the subject to be treated with the modified cells. In some embodiments, however, the cells are allogeneic, i.e., isolated from an HLA-matched or HLA-compatible, or otherwise suitable, donor.

In some embodiments, cells are harvested from the subject and modified according to the methods disclosed herein, which can include selecting certain cell types, optionally expanding the cells and optionally culturing the cells, and which can additionally include selecting cells that contain a transgene integrated into the targeted locus. In particular embodiments, such modified cells are then reintroduced into the subject.

Further disclosed herein are methods of using said nuclease systems to produce the modified host cells described herein, comprising introducing into a mammalian cell: (a) an RNP comprising a Cas nuclease such as Cas9 and an sgRNA specific to a locus of interest, (b) a DNA-PK inhibitor, and (c) a homologous donor template or vector as described herein. Each component can be introduced into the cell directly or can be expressed in the cell by introducing a nucleic acid encoding the components of said one or more nuclease systems.

In some embodiments, the present methods target integration of a functional cDNA at the corresponding endogenous locus or at a safe harbor locus in a host cell ex vivo. In some embodiments, the methods target the modification of a genomic sequence, e.g., the alteration of a genomic sequence, or the introduction of a deletion or insertion, at an endogenous locus. Such methods can further comprise (a) optionally expanding said cells, and/or (b) optionally culturing the cells.

In any of these methods, the nuclease can produce one or more single stranded breaks within the locus of interest, or a double stranded break within the locus of interest. In these methods, the locus is modified by homologous recombination with said donor template or vector to result in insertion of the transgene into the locus. The methods can further comprise (c) selecting cells that contain the transgene integrated into the locus of interest.

### 7. Detecting DNA-PK Inhibitor Activity

The activity of a DNA-PK inhibitor and/or the efficacy of the present methods can be assessed in any of a number of ways. For example, the activity of a DNA-PK inhibitor can be assessed by measuring the rate of HDR in cells such as CD34⁺ HSPCs, e.g., the rate of integration of a cDNA at genomic loci such as HBB, CCR5, IL2RG, HBA1, CFTR, or STING1 when a DNA-PK inhibitor is introduced together with an sgRNA, RNA-guided nuclease, and homologous donor template. In some embodiments, the rate of HDR in such cells is increased by at least about 10%, 20%, 30%, 40%, 50%, or more relative to the rate in equivalent cells but in the absence of DNA-PK inhibitor. In some embodiments, the activity of a DNA-PK inhibitor can be assessed by measuring the rate of NHEJ or indels in cells such as CD34⁺ HSPCs. In some embodiments, the rate of indels is decreased by at least about 10%, 20%, 30%, 40%, 50%, 60%, 70%, or more relative to the rate in equivalent cells in the absence of DNA-PK inhibitor. In some embodiments, the rate of HDR is increased in cells modified (e.g., according to the methods herein) with use of a DNA-PK inhibitor by at least about 10%, 20%, 30%, 40%, 50%, or more (i.e., relative to the rate in equivalent cells modified without use of a DNA-PK inhibitor) with few or no detectable indels.

In some embodiments, the activity of a DNA-PK inhibitor is assessed by determining the MOI for a viral vector comprising a homologous donor template that is required to achieve a given level of HDR. For example, in some embodiments, the presence of DNA-PK inhibitor can allow a decrease in the MOI used of, e.g., 1-fold, 2-fold, 3-fold, 4-fold, or more, while still maintaining similar rates of HDR as compared to in an equivalent cell in the absence of DNA-PK inhibitor. In some embodiments, the activity of a DNA-PK inhibitor can be assessed by determining, e.g., the ability of modified cells to achieve a given rate of engraftment in animal models. For example, the presence of DNA-PK inhibitor can allow the use of an MOI that is, e.g., 1-fold, 2-fold, 3-fold, 4-fold, or more lower than the MOI needed in the absence of DNA-PK inhibitor, to achieve a given rate of engraftment. In some embodiments, the activity of a DNA-PK inhibitor is assessed by determining the amount of sgRNA and/or RNA-guided nuclease that is required to achieve a given level of HDR. For example, in some embodiments, the presence of DNA-PK inhibitor can allow a decrease in the amount of sgRNA and/or RNA-guided nuclease used of, e.g., 1-fold, 2-fold, 3-fold, 4-fold, or more, while still maintaining similar rates of HDR as compared to in an equivalent cell in the absence of DNA-PK inhibitor. As another example, the presence of DNA-PK inhibitor can allow the use of an amount of sgRNA and/or RNA-guided nuclease that is, e.g., 1-fold, 2-fold, 3-fold, 4-fold, or more lower than the amount needed in the absence of DNA-PK inhibitor, to achieve a given rate of engraftment.

In some embodiments, the activity of a DNA-PK inhibitor is assessed by comparing the ability of a particular gRNA to induce desired edits (e.g., HDR) at its targeted locus. In some embodiments (e.g., as demonstrated in Example 2 herein), a gRNA that induces low to no indels at its target locus in the absence of DNA-PK inhibitor is able to induce high frequency of HDR at its target locus in the presence of DNA-PK inhibitor. In some embodiments, DNA-PK inhibitor treatment increases the number of sgRNAs that can be used for HDR because of the ability to promote high HDR frequency induced by sgRNAs that are seemingly inactive in the absence of DNA-PK inhibitor.

The activity of a DNA-PK inhibitor can also be assessed in cells by examining, e.g., any known activity of DNA-PK, such as DNA DSB repair. In some embodiments, a DNA-PK inhibitor can reduce a DNA-PK activity by at least about 10%, 20%, 30%, 40%, 50%, 60%, 70%, or more relative to the activity in the absence of DNA-PK inhibitor.

The activity of a DNA-PK inhibitor can also be assessed by examining the impact of HDR on the modified cells. In some embodiments, the methods provided herein decrease the toxicity of HDR in modified cells and lead to, e.g., increased viability and/or improved function of modified cells. In some embodiments, use of a DNA-PK inhibitor in the methods herein can increase the viability and/or the function of the modified cells by at least about 10%, 20%, 30%, 40%, 50%, 60%, 70%, or more relative to cells modified without use of a DNA-PK inhibitor.

### 8. Methods of Treatment

In some embodiments, following the modification of the genome in cells from a subject using the herein-described methods, and, e.g., confirming expression of a protein encoded by an introduced cDNA, a plurality of modified cells can be reintroduced into the subject, such that they can repopulate and differentiate, and due to the expression of the integrated cDNA (or other genetic modification), can improve one or more abnormalities or symptoms in the subject with the genetic disorder. In some embodiments, the cells are expanded, selected, and/or induced to undergo differentiation, prior to reintroduction into the subject.

Disclosed herein, in some embodiments, are methods, including therapeutic methods and methods of administration. Although the descriptions of methods provided herein are principally directed to administration to humans, it will be understood by the skilled artisan that they are generally suitable for administration to any animals.

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously.

In some embodiments, a subject will undergo a conditioning regime before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007); Palchaudari et al., 34(7) Nature Biotechnology 738-745 (2016); Chhabra et al., 10:8(351) Science Translational Medicine 351ra105 (2016)) and CAR T-mediated conditioning (see, e.g., Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

The present disclosure additionally provides methods of administering modified host cells in accordance with the disclosure to a subject in need thereof. Pharmaceutical compositions including the modified host cell may be administered to a subject using any amount and any route of administration effective for preventing, treating, or managing the condition in question. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed; the specific composition employed; the age, body weight, general health, sex and diet of the patient; the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more modified cells to the subject, or any amount sufficient to obtain the desired therapeutic or prophylactic, effect. The desired dosage of the modified host cells of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

### 9. Examples

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. AZD7648 Enhances Gene Targeting Efficiency Across Different Genomic Loci

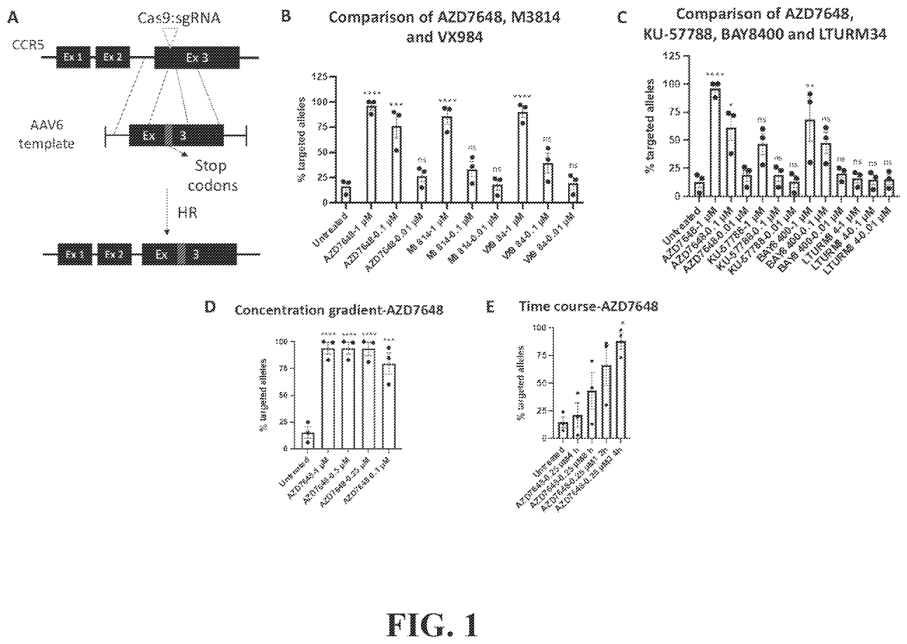

AZD7648 and VX984 are more selective at inhibition of DNA-PK than M3814 based on the IC50 values against the other targets belonging to the PI3K family, ATM, ATR and mTOR kinases²² (Table 1). We tested the effect of these three compounds and multiple other DNA-PK inhibitors (KU57788, BAY8400 and LTURM34) at three different concentrations (1, 0.1 and 0.01 μM) on gene targeting efficiency at CCR5 locus for integration of a short sequence (two stop codons in tandem) using Cas9 RNP and AAV6 based platform in hPSCs (FIG. 1A-C). Among the tested compounds, only AZD7648 significantly enhanced gene targeting at two of the three tested concentrations (1 and 0.1 μM) while the remaining compounds had a significant effect only at 1 μM. Thus, AZD7648 is the most potent among the tested DNA-PK inhibitors for enhancing gene targeting efficiency. For the AZD7648 treatment, we then performed concentration gradient (1, 0.5, 0.25 and 0.1 μM), time course analysis (4, 8, 12 and 24 h) and found that the optimal concentration and incubation time are 0.25 μM and 24 h respectively (FIGS. 1D-E).

TABLE 1
IC50 values of different small molecule inhibitors of DNA-PK
against various targets based on a previous study²²

| Targets | M3814 (IC50) | | AZD7648 (IC50) | | VX984 (IC50) | |
|---|---|---|---|---|---|---|
| DNA-PKcs | 43 | nM | 91.3 | nM | 115 | nM |
| ATM | >30 | μM | 17.93 | μM | >30 | μM |
| ATR | >30 | μM | >29.77 | μM | >30 | μM |
| PI3Kα | 0.8 | μM | >8.03 | μM | >20 | μM |
| PI3Kβ | 0.17 | μM | >30 | μM | >30 | μM |
| PI3Kγ | 1.59 | μM | 1.37 | μM | 7.1 | μM |
| PI3Kδ | 0.35 | μM | >30 | μM | >30 | μM |
| mTOR | 0.55 | μM | >30 | μM | >30 | μM |

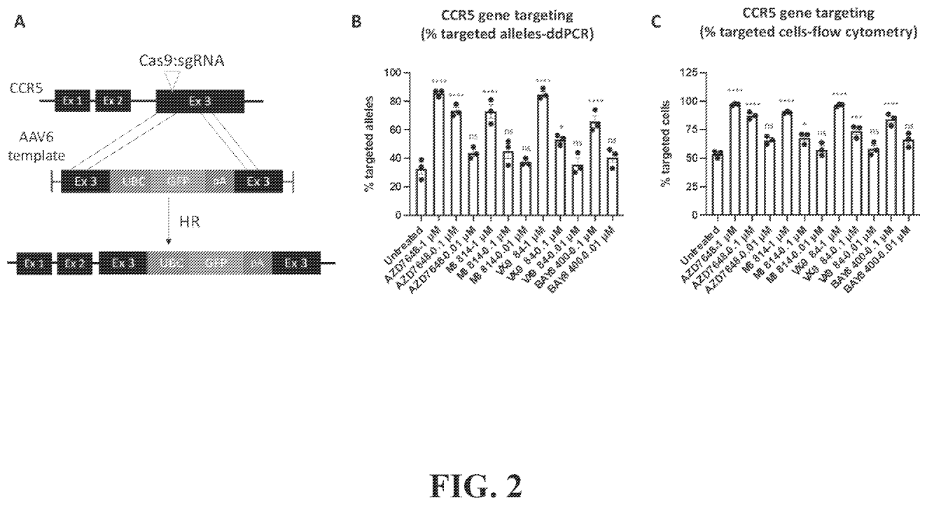

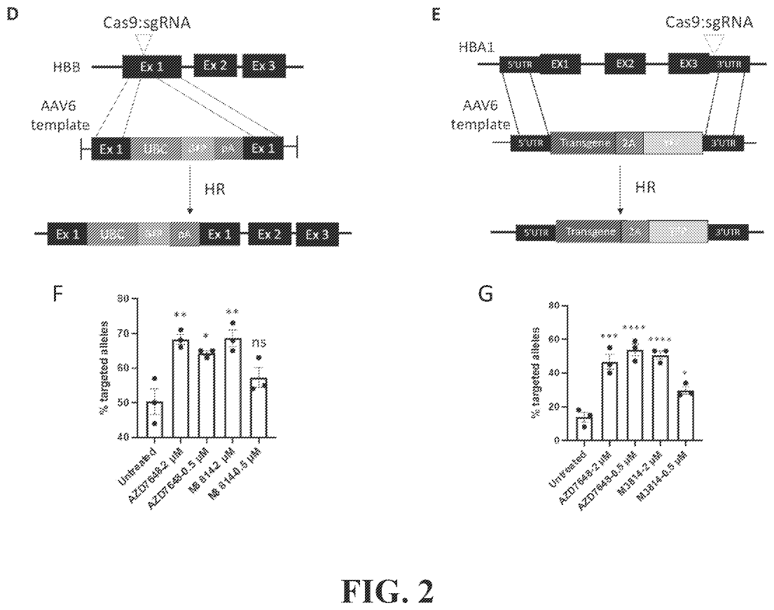

Next, we tested the effect of different DNA-PK inhibitors (AZD7648, M3814, VX984 and BAY8400) on gene targeting efficiency at CCR5 locus for integration of a large sequence (UBC-GFP-bGHpA-2.6 kb length) at three different concentrations (1, 0.1 and 0.01 μM) using Cas9 RNP and AAV6 based platform in hPSCs (FIG. 2A-C). Gene targeting efficiency was measured by droplet digital PCR (ddPCR) and flow cytometry (for GFP) to measure the allelic targeting efficiency (FIG. 2B) and percentage of targeted cells (FIG. 2C) respectively. Among the tested compounds, AZD7648 and VX984 significantly enhanced gene targeting at two of the three tested concentrations (1 and 0.1 μM) while BAY8400 had a significant effect at 0.1 μM but was very toxic at 1 μM (data not shown). Thus, AZD7648 is potent at enhancing gene targeting efficiency for targeted integration of both short and long sequences. We then tested AZD7648 and M3814 treatment for gene targeting at HBB and HBA1 loci at concentrations of 2 μM and 0.5 μM in three different hPSCs (FIG. 2D-E). The treatment was carried out for 24 hours post gene targeting and the efficiency was calculated 1 week post gene targeting using ddPCR analysis. At 2 μM, both AZD7648 and M3814 significantly enhanced the gene targeting at both loci up to 4-fold when compared to the untreated control. At 0.5 μM, only AZD7648 enhanced the gene targeting significantly while M3814 had a less prominent effect (FIGS. 2D-E). Thus, AZD7648 is more potent than M3814 for enhancing gene targeting efficiency.

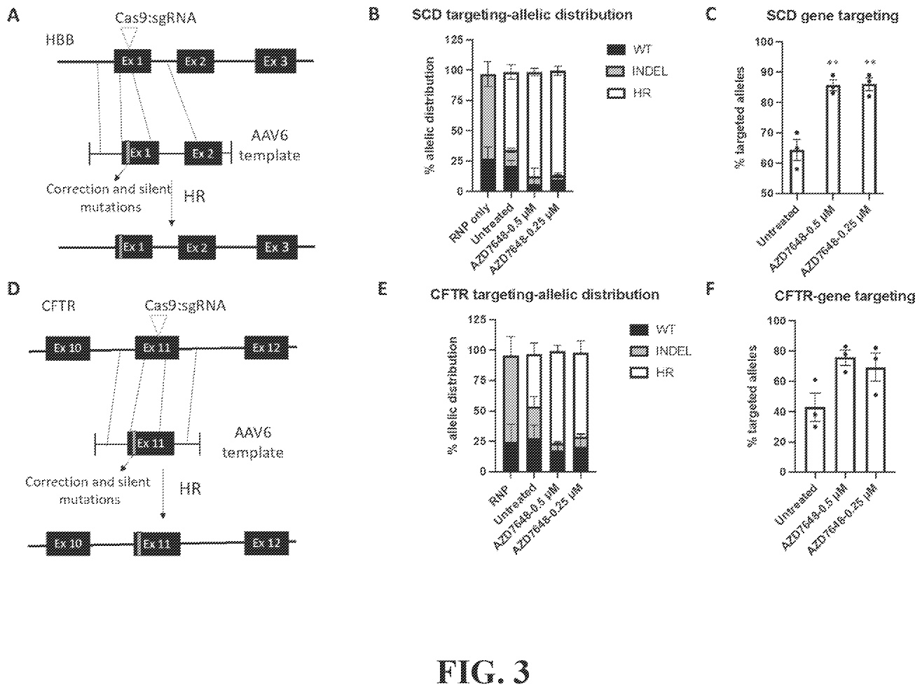

To confirm the applicability of this approach for correction of genetic disease-associated point mutations, we assessed the effect of AZD7648 treatment on RNP/AAV6 based editing of sickle cell disease (SCD) and cystic fibrosis (CF) mutations in WT hPSC. For editing the SCD E6V mutation in HBB gene (FIG. 3A), AZD7648 treatment (0.5 and 0.25 μM) reduced the INDEL frequency compared to the untreated cells (FIG. 3B) and correspondingly the gene targeting efficiency was improved significantly (FIG. 3B-C). Similarly, for editing the CF AF508 mutation in CFTR gene (FIG. 3D), AZD7648 treatment (0.5 and 0.25 μM) reduced the INDEL frequency (FIG. 3E) and increased the gene targeting efficiency (FIG. 3E-F). Thus, AZD7648 treatment enhances the gene targeting efficiency for editing the SCD and CF disease-associated mutations.

### Example 2. AZD7648 Enhances Gene Targeting Efficiency with a Seemingly Inactive gRNA

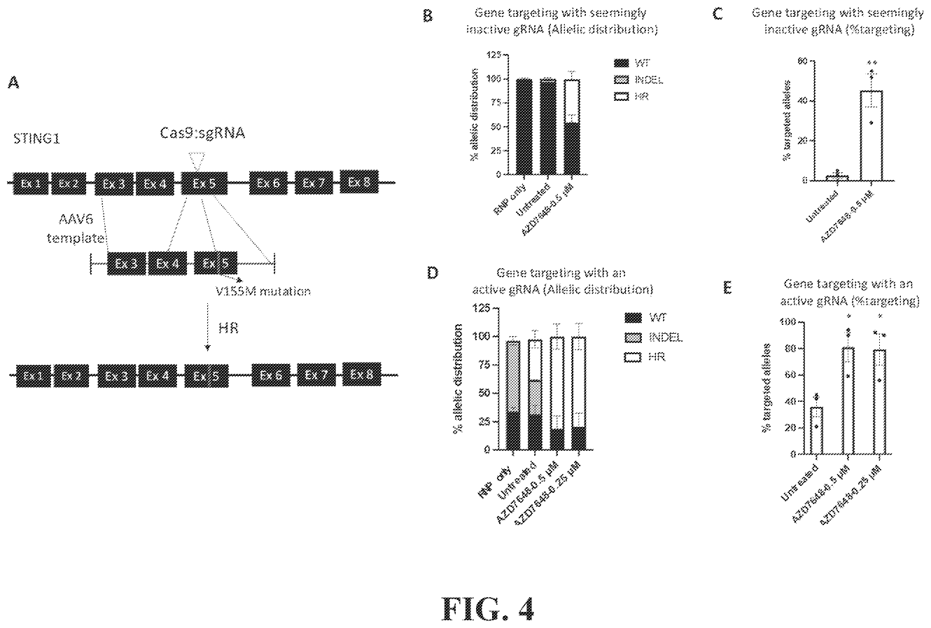

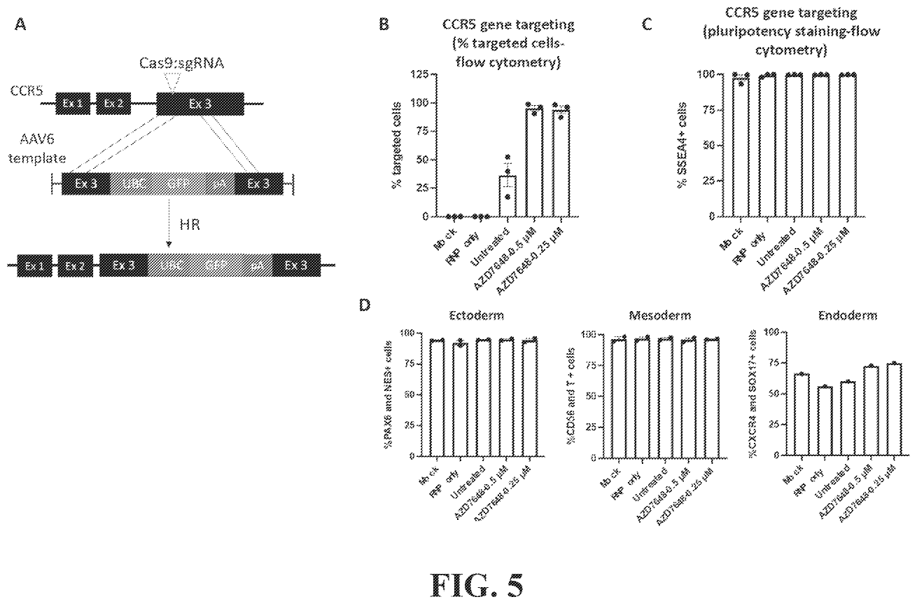

Next, we tested the feasibility of enhancing gene targeting with AZD7648 treatment using a seemingly inactive gRNA targeting STING1 locus in hPSCs for introduction of V155M mutation associated with STING-associated vasculopathy with onset in infancy (SAVI) disease (FIG. 4A). Upon Cas9 RNP nucleofection, the INDEL frequency at STING1 locus was 1-2% in three different hPSCs indicating that the gRNA is seemingly inactive. AAV6 donor transduction following RNP nucleofection (untreated) resulted in average gene targeting of 3%. Treatment with AZD7648 (0.5 μM) resulted in gene targeting frequency of up to 50% without indels (FIG. 4B-C). With an active gRNA that induces INDEL at more than 60% efficiency following RNP nucleofection, AZD7648 treatment (0.5 and 0.25 μM) improves HR efficiency by 2 to 3-fold without INDELs following AAV6 donor transduction. In comparison, the untreated cells had around 30% INDELs (FIG. 4D-E). Thus, AZD7648 treatment could be used to enhance gene targeting efficiency with both seemingly inactive gRNAs and active gRNAs.

### Example 3. Pluripotency is Maintained in hPSC Gene Targeted with AZD7648

To confirm the maintenance of pluripotency in hPSC gene targeted with AZD7648 treatment, we assessed the pluripotency marker expression and trilineage (three-germ layer) differentiation potential. hPSC were gene targeted at CCR5 locus for integration of UBC-GFP-bGHpA (FIG. 5A) with and without AZD7648 treatment (0.5 and 0.25 μM). AZD7648 treatment enhanced gene targeting efficiency by about 3-fold compared to untreated cells as measured by the frequency of GFP+ cells (FIG. 5B). Gene targeted hPSCs were assessed for pluripotency marker, SSEA4 expression using flow cytometry and we found that the expression of SSEA4 was maintained in cells gene targeted with or without AZD7648 (FIG. 5C). We then assessed the trilineage differentiation potential of the gene targeted hPSC, using a commercially available kit. Post-differentiation, we assessed the efficiency of differentiation into ectoderm, mesoderm and endoderm by flow cytometry analysis for expression of corresponding markers (PAX6 and NES for ectoderm; CD56 and T for mesoderm; SOX17 and CXCR4 for endoderm). Trilineage differentiation potential was unaffected in hPSCs differentiated with and without AZD7648 treatment (FIG. 5 D). Thus, pluripotency is maintained in hPSCs gene targeted with AZD7648 treatment.

### Example 4. AZD7648 Treatment Maintains High Gene Targeting Efficiency with Reduced Amounts of Cas9 RNP and AAV6 Donor

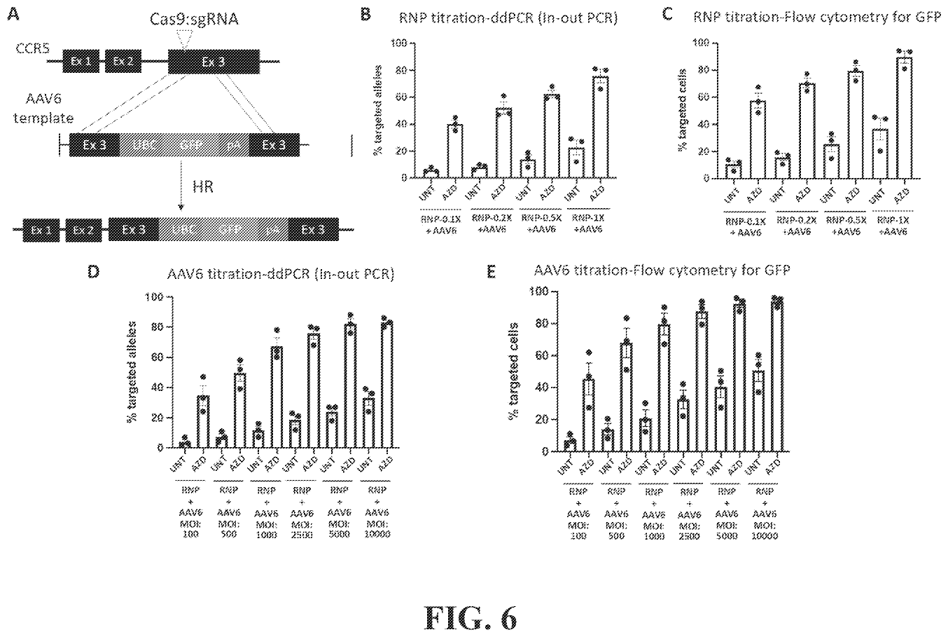

Next, we tested whether high gene targeting efficiency can be maintained with AZD7648 treatment using reduced amounts of Cas9 RNP and AAV6. We assessed this for gene targeting at CCR5 locus for integration of UBC-GFP-bGHpA sequence in hPSCs (FIG. 6A). Cas9 RNP amount was titrated down by 2-, 5- and 10-fold (0.5×, 0.2× and 0.1×) and gene targeting efficiency was assessed with and without AZD7648 treatment by ddPCR and flow cytometry analysis (FIG. 6B-C). Although gene targeting efficiency decreased gradually with titrating down Cas9 RNP amount, the efficiency remained higher at all tested doses with AZD7648 treatment as compared to untreated cells with full amount of RNP (FIG. 6B-C). Similarly, we tested the effect of titrating down the amount of AAV6 donor between an MOI of 100 to 10000 on gene targeting with AZD7648. Based on ddPCR and flow cytometry analysis, AZD7648 treated cells showed higher gene targeting efficiency even at 500 MOI as compared to the untreated cells at the highest MOI of 10000 (FIG. 6D-E). Thus, AZD7648 treatment can maintain high gene targeting efficiency even with reduced amounts of Cas9 RNP or AAV6 donor.

### Example 5. AZD7648 Enhances Gene Targeting Efficiency in Human CD34+ HSPCs

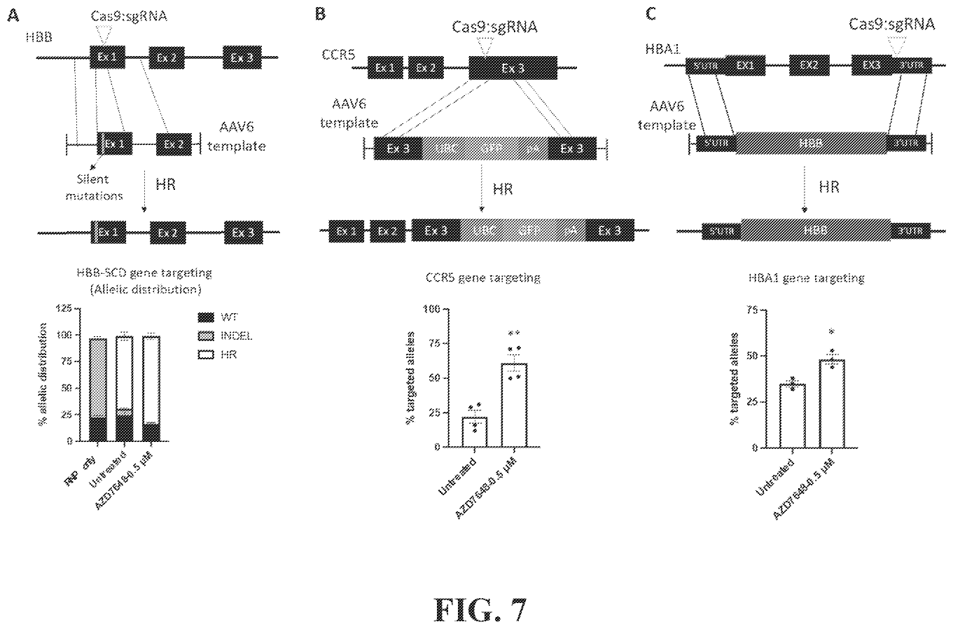

We tested the effect of AZD7648 treatment on gene targeting efficiency in human CD34+ hematopoietic stem and progenitor cells (HSPCs). First, we tested the gene targeting efficiency for editing the SCD mutation (E6V) in cord blood-derived CD34+ HSPCs with and without AZD7648 treatment. Although, the untreated cells showed high gene targeting efficiency of around 70%, there was a small frequency of alleles with INDELs. With AZD7648 treatment, gene targeting efficiency was improved to around 80% with almost no INDELs (FIG. 7A). Next, we tested the effect of AZD7648 on gene targeting efficiency at CCR5 locus which is a safe harbor for integration of therapeutic transgenes. AZD7648 treatment significantly enhanced gene targeting at CCR5 locus for integration of UBC-GFP-bGHpA sequence by around 3-fold compared to that of untreated cells (FIG. 7B). We then tested the effect of AZD7648 treatment for gene replacement at HBA1 locus to introduce HBB gene as a therapeutic strategy for β-thalassemia. ddPCR analysis showed that allelic gene targeting efficiency can be improved by around 1.4-fold with AZD7648 treatment (FIG. 7C). Thus, AZD7648 enhances the efficiency of therapeutically relevant gene targeting at HBB, CCR5 and HBA1 loci in human HSPCs.

### Example 6. Gene Targeting in Human T and B Cells is Enhanced by AZD7648 Treatment

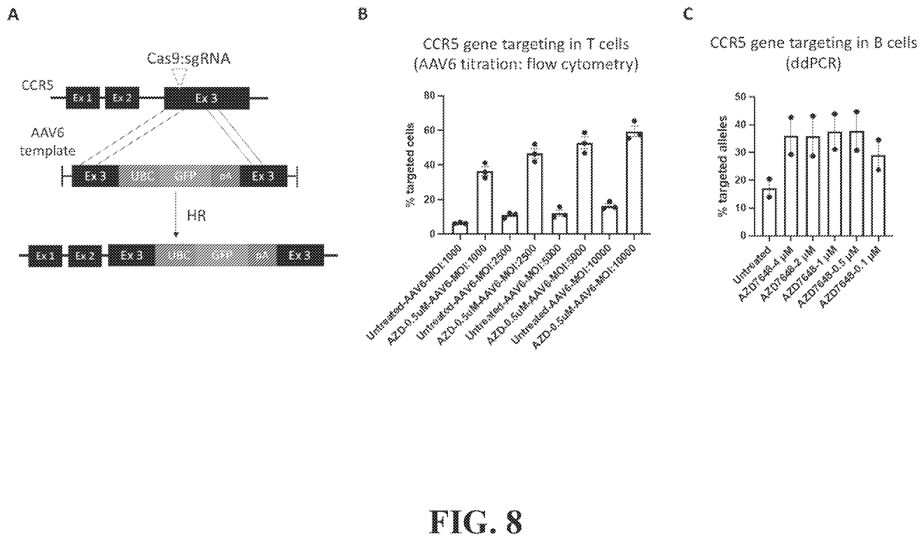

Next, we tested AZD7648 treatment for improving gene targeting at CCR5 locus (FIG. 8A) in human B and T cells. In T cells, we tested different amounts of AAV6 (MOI. 1000, 2500, 5000 and 10000) for gene targeting with and without AZD7648 treatment. Based on flow cytometry for GFP, we found that AZD7648 treatment enhances gene targeting efficiency across all tested AAV6 MOIs when compared to untreated cells even at highest MOI of 10000 (FIG. 8B). For B cells, we tested AZD7648 treatment at different concentrations (4, 2, 1, 0.5 and 0.1 μM) for gene targeting at CCR5 locus. ddPCR analysis showed that AZD7648 treatment enhances gene targeting by about 2-fold at different concentrations (FIG. 8C). Thus, AZD7648 treatment enhances gene targeting in human T and B cells.

### Example 7. AZD7648 Enhances Correction of CF Mutation in HBECs

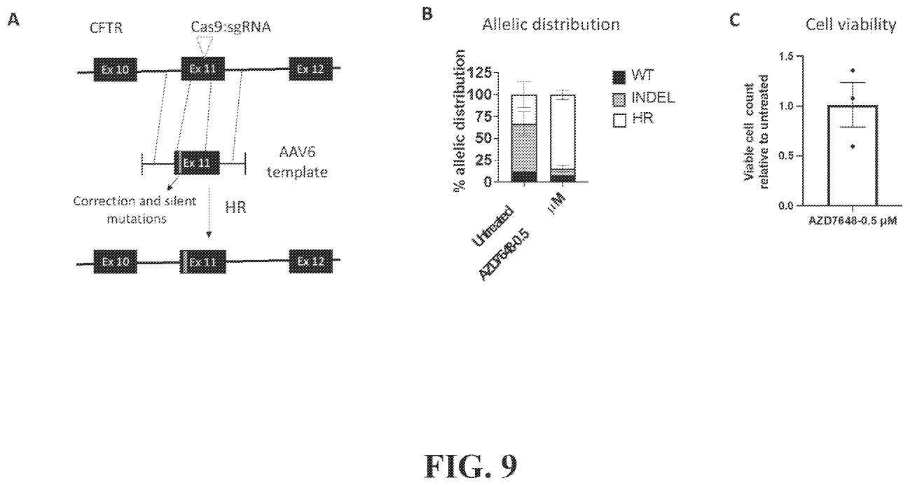

Next, we tested the effect of AZD7648 treatment for gene targeting to correct the cystic fibrosis (CF) associated AF508 mutation in exon 11 of CFTR gene in CF patient-derived human bronchial epithelial cells (HBECs) (FIG. 9A). Based on ICE analysis, we found that HBECs treated with AZD7648 showed significantly higher gene targeting efficiency with much lower frequency of alleles with INDELs as compared to the untreated cells (FIG. 9B). Importantly, cell viability assessment post-gene targeting showed that AZD7648 treatment did not affect the percentage of viable cells relative to untreated control (FIG. 9C). Thus, AZD7648 treatment improves gene targeting efficiency for the correction of CF mutation in patient-derived HBECs without affecting the cell viability.

### REFERENCES

1. Porteus M H. A New Class of Medicines through DNA Editing. N Engl J Med. 2019; 380(10):947-959. doi:10.1056/NEJMra1800729
2. Uddin F, Rudin C M, Sen T. CRISPR Gene Therapy: Applications, Limitations, and Implications for the Future. Front Oncol. 2020; 10:1387. doi:10.3389/fonc.2020.01387
3. Doudna J A, Charpentier E. Genome editing. The new frontier of genome engineering with CRISPR-Cas9. Science. 2014; 346(6213):1258096. doi:10.1126/science.1258096
4. Hsu P D, Lander E S, Zhang F. Development and applications of CRISPR-Cas9 for genome engineering. Cell. 2014; 157(6):1262-1278. doi:10.1016/j.cell.2014.05.010
5. Kanaar R, Hoeijmakers J H, van Gent D C. Molecular mechanisms of DNA double strand break repair. Trends Cell Biol. 1998; 8(12):483-489. doi:10.1016/s0962-8924(98)01383-x
6. West S C, Chappell C, Hanakahi L A, Masson J Y, McIlwraith M J, Van Dyck E. Double-strand break repair in human cells. Cold Spring Harb Symp Quant Biol. 2000; 65:315-321. doi:10.1101/sqb.2000.65.315
7. Betermier M, Bertrand P, Lopez B S. Is non-homologous end-joining really an inherently error-prone process?PLoS Genet. 2014; 10(1):e1004086. doi:10.1371/journal.pgen.1004086
8. Suzuki K, Tsunekawa Y, Hernandez-Benitez R, et al. In vivo genome editing via CRISPR/Cas9 mediated homology-independent targeted integration. Nature. 2016; 540(7631):144-149. doi:10.1038/nature20565
9. Dever D P, Bak R O, Reinisch A, et al. CRISPR/Cas9 β-globin gene targeting in human haematopoietic stem cells. Nature. 2016; 539(7629):384-389. doi:10.1038/nature20134
10. Martin R M, Ikeda K, Cromer M K, et al. Highly Efficient and Marker-free Genome Editing of Human Pluripotent Stem Cells by CRISPR-Cas9 RNP and AAV6 Donor-Mediated Homologous Recombination. Cell Stem Cell. 2019; 24(5):821-828.e5. doi:10.1016/j.stem.2019.04.001
11. Martin R M, Fowler J L, Cromer M K, et al. Improving the safety of human pluripotent stem cell therapies using genome-edited orthogonal safeguards. Nat Commun. 2020; 11(1):2713. doi:10.1038/s41467-020-16455-7
12. Cromer M K, Camarena J, Martin R M, et al. Gene replacement of α-globin with (3-globin restores hemoglobin balance in β-thalassemia-derived hematopoietic stem and progenitor cells. Nat Med. 2021; 27(4):677-687. doi:10.1038/s41591-021-01284-y
13. Lattanzi A, Camarena J, Lahiri P, et al. Development of β-globin gene correction in human hematopoietic stem cells as a potential durable treatment for sickle cell disease. Sci Transl Med. 2021; 13(598):eabf2444. doi:10.1126/scitranslmed.abf2444
14. Vakulskas C A, Dever D P, Rettig G R, et al. A high-fidelity Cas9 mutant delivered as a ribonucleoprotein complex enables efficient gene editing in human hematopoietic stem and progenitor cells. Nat Med. 2018; 24(8):1216-1224. doi:10.1038/s41591-018-0137-0
15. Wiebking V, Lee C M, Mostrel N, et al. Genome editing of donor-derived T-cells to generate allogenic chimeric antigen receptor-modified T cells: Optimizing αβ T cell-depleted haploidentical hematopoietic stem cell transplantation. Haematologica. 2021; 106(3):847-858. doi:10.3324/haematol.2019.233882
16. Vaidyanathan S, Salahudeen A A, Sellers Z M, et al. High-Efficiency, Selection-free Gene Repair in Airway Stem Cells from Cystic Fibrosis Patients Rescues CFTR Function in Differentiated Epithelia. Cell Stem Cell. 2020; 26(2):161-171.e4. doi:10.1016/j.stem.2019.11.002
17. Vaidyanathan S, Baik R, Chen L, et al. Targeted replacement of full-length CFTR in human airway stem cells by CRISPR-Cas9 for pan-mutation correction in the endogenous locus. Mol Ther J Am Soc Gene Ther. 2022; 30(1):223-237. doi:10.1016/j.ymthe.2021.03.023
18. Fu Y W, Dai X Y, Wang W T, et al. Dynamics and competition of CRISPR-Cas9 ribonucleoproteins and AAV donor-mediated NHEJ, MMEJ and HDR editing. Nucleic Acids Res. 2021; 49(2):969-985. doi:10.1093/nar/gkaa1251
19. Ray U, Raghavan S C. Modulation of DNA double-strand break repair as a strategy to improve precise genome editing. Oncogene. 2020; 39(41):6393-6405. doi:10.1038/s41388-020-01445-2
20. Mohiuddin I S, Kang M H. DNA-PK as an Emerging Therapeutic Target in Cancer. Front Oncol. 2019; 9:635. doi:10.3389/fonc.2019.00635
21. Riesenberg S, Chintalapati M, Macak D, Kanis P, Maricic T, Pääbo S. Simultaneous precise editing of multiple genes in human cells. Nucleic Acids Res. 2019; 47(19):e116. doi:10.1093/nar/gkz669
22. Fok J H L, Ramos-Montoya A, Vazquez-Chantada M, et al. AZD7648 is a potent and selective DNA-PK inhibitor that enhances radiation, chemotherapy and olaparib activity. Nat Commun. 2019; 10(1):5065. doi:10.1038/s41467-019-12836-9
23. Khan A J, Misenko S M, Thandoni A, et al. VX-984 is a selective inhibitor of non-homologous end joining, with possible preferential activity in transformed cells. Oncotarget. 2018; 9(40):25833-25841. doi:10.18632/oncotarget.25383
24. Damia G. Targeting DNA-PK in cancer. Mutat Res. 2020; 821:111692. doi:10.1016/j.mrfmmm.2020.111692

### Example 8. DNA-PKcs Inhibition Results in Higher Genome Editing Frequencies of HDR Compared to INDELs in Human Primary Cells

### Abstract

Nuclease-based genome editing can result in either site-specific insertions/deletions (INDELs) or targeted integrations depending on which pathway of natural DNA double-strand break repair is harnessed: non-homologous end-joining (NHEJ), microhomology mediated end-joining (MMEJ) or homology-directed repair (HDR). While highly active targeted integration systems have been developed, the use of targeted integration for either research or therapeutic applications would be greatly expanded if methods to further increase HDR and minimize INDELs were developed. We screened various small molecule inhibitors of DNA-dependent protein kinase catalytic subunit (DNA-PKcs), a key protein in NHEJ, to improve the HDR efficiency and identified AZD7648 as the most potent compound. The use of AZD7648 resulted in significant increases in HDR (up to 50-fold in some examples) with concomitant decreases in INDELs across different genomic loci in various therapeutically relevant primary human cell types such as pluripotent stem cells, hematopoietic stem and progenitor cells, T cells and bronchial epithelial cells. In all cases, the ratio of HDR to INDEL markedly increased and in certain situations, INDEL-free, high frequency (>50%) targeted integration was achieved. Targeted integration with AZD7648 treatment could improve the therapeutic efficacy of cell-based therapies and broaden the use of targeted integration as a research tool as it now enables applications of genome editing that might not have been previously possible.

### Main

Genome editing is a method to change the nucleotide sequence of a cell with single nucleotide precision. The most well-developed method of genome editing is using an engineered nuclease to create a site-specific DNA double-strand break (DSB) in the genome25, 26. CRISPR-Cas9 is the most widely used engineered nuclease system for genome editing due to its ease of design, high activity, and high specificity. CRISPR-Cas9 genome editing is a two-component system consisting of a guide RNA (gRNA) that is complementary in sequence to the genomic target site, which brings the Cas9 nuclease to create a precise DSB²⁷⁻³¹. There are redundant cellular mechanisms to repair the induced DSB³². DSB can be repaired by non-homologous end-joining (NHEJ) pathway, a generally accurate form of repair that occasionally can result in small insertions/deletions (INDELs) often of a single nucleotide at the break site^{33,34}. If the DSB is repaired by microhomology mediated end-joining (MMEJ), small to large INDELs are created³⁵⁻³⁷. Finally, homology-directed repair (HDR) involves the use of either endogenous or exogenous homologous donor template sequence to precisely repair the DSB. Upon providing an exogenous donor template, HDR pathway can be harnessed for targeted integration of single nucleotide changes or of several thousand base pairs^{32,38}. HDR-mediated genome editing is currently the most flexible method of creating the widest variety of changes to the genome of a cell and has now entered clinical studies³⁹.

One of the hallmarks of nuclease-based genome editing is that in a population of cells, a mixture of INDELs and HDR-mediated targeted insertions occurs. Moreover, while HDR to INDEL ratios of 1:1 or greater can be achieved, the frequency of INDELs is often greater than that of HDR^{32,40}. An important advance in genome editing would be to develop methods in which HDR is significantly more frequent than INDELs and ideally INDEL-free targeted integration might be achieved. During genome editing, HDR-based targeted integration has been shown to outcompete the MMEJ-based INDELs, but not the NHEJ-based INDELs^{40, 41}. This is likely due to the NHEJ pathway being active in all phases of the cell cycle while HDR and MMEJ are active in S and G2 phases of the cell cycle^{42, 43}. Thus, different methods for inhibition of the NHEJ pathway are being explored for enhancing the frequency of HDR-based gene targeting/targeted integration⁴⁴⁻⁴⁶.

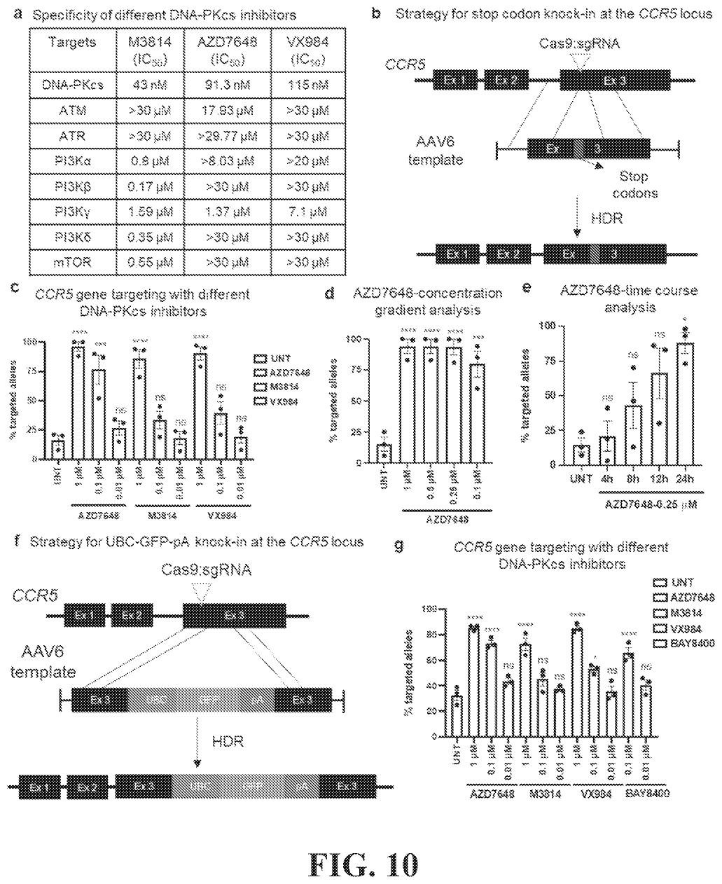

The NHEJ pathway is initiated following the binding of Ku70/80 protein to the ends of the DSB³⁵, 47 Ku70/80 then recruits the catalytic subunit of DNA-dependent protein kinase (DNA-PKcs) to form the DNA-dependent protein kinase complex. DNA-PKcs is activated through autophosphorylation and is critical for progression of the NHEJ pathway⁴⁸. Subsequently, the XRCC4, XLF and DNA ligase IV complex is recruited to ligate the broken ends⁴⁹. Inhibition of Ku or DNA ligase IV through shRNA/siRNA or small molecules has been shown to moderately improve HDR efficiency by reducing the levels of NHEJ⁵⁰⁻⁵³. In addition, peptide-based inhibition of 53BP1 (a protein that promotes the NHEJ pathway) can also result in increased frequencies of HDR-mediated genome editing⁵⁴. A recent study has shown that introduction of a catalytically inactive mutation in DNA-PKcs leads to high levels of HDR-based gene targeting following gene editing with single-stranded oligodeoxynucleotide (ssODN) donor and Cas9-gRNA in human induced pluripotent stem cells (iPSCs)⁵⁵. This enhancement in gene targeting could also be recapitulated with transient inhibition of DNA-PKcs using a small molecule compound, M3814^{40,55}. Although, M3814 is a potent DNA-PKcs inhibitor, it has been shown to be less selective than other small molecule DNA-PKcs inhibitors⁵⁶. At the concentrations used for gene targeting, M3814 can inhibit various kinases of the PI3K family and the mTOR kinase potentially causing cellular toxicity (FIG. 10a)^{40,55}. Thus, DNA-PKcs inhibitors with higher specificity might result in further improvements in gene targeting efficiencies with mitigated cellular toxicity.

We screened various selective small molecule DNA-PKcs inhibitors for enhancing gene targeting efficiency using the Cas9 ribonucleoprotein (RNP) and adeno-associated virus serotype 6 (AAV6) HDR donor delivery in human pluripotent stem cells (PSCs)⁵⁷. From this screening, AZD7648 was identified as the most potent compound for enhancing gene targeting efficiency. AZD7648 was not only more potent than M3814 for improving gene targeting but has also been shown to be a more selective DNA-PKcs inhibitor than M3814 (FIG. 10a)⁵⁶. AZD7648 treatment significantly enhanced gene targeting efficiency at different genomic loci for integration of both short and large sequences in various therapeutically relevant human primary cells such as PSCs, hematopoietic stem and progenitor cells (HSPCs), T cells and human bronchial epithelial cells (HBECs). With AZD7648 treatment, we achieved high levels of gene targeting with low to no INDELs in most cases. Allelic gene targeting efficiency reached as high as 90% in some cases. Remarkably, AZD7648 could turn a gRNA that generated very few INDELs when delivered as an RNP alone without AZD7648 into one that generated ˜50% targeted integrations without detectable INDELs when combined with AAV6 HDR donor and AZD7648. The use of AZD7648 resulted in the flipping of the HDR to INDEL ratio from 1:1 to >5-100:1 in most circumstances. Thus, AZD7648 treatment can broadly enhance the ex vivo targeted integration frequency in primary cells thereby improving the efficacy of ex vivo gene targeted cell-based therapies and expanding the application of targeted integration for research purposes.

### Results

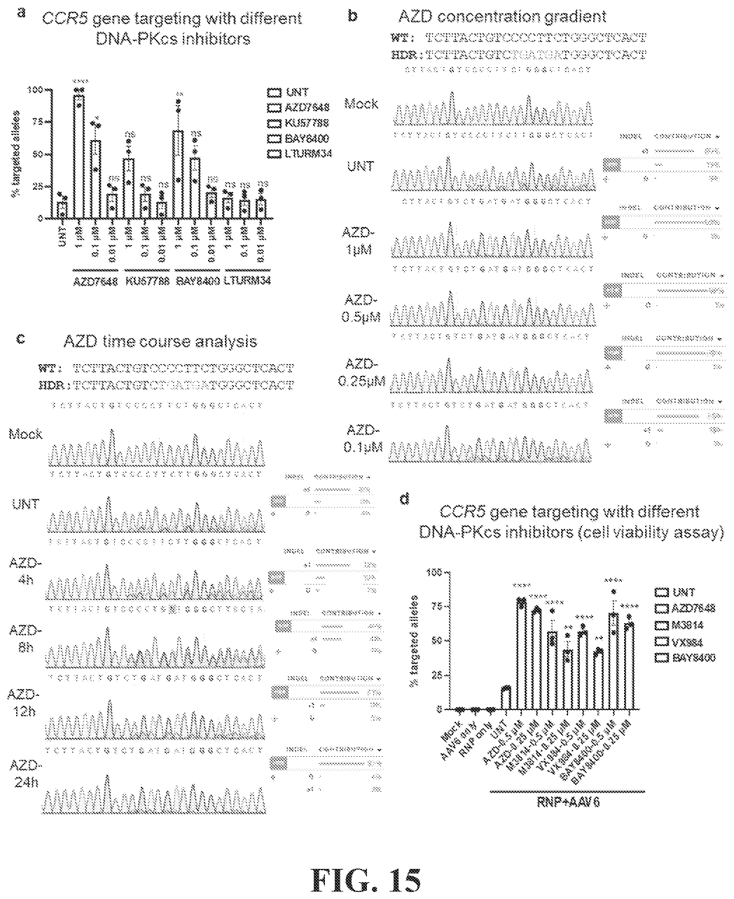

AZD7648 is the most potent DNA-PKcs inhibitor for enhancing gene targeting efficiency in PSC. Small molecule DNA-PKcs inhibitors, AZD7648 and VX984 have a better selectivity profile than M3814 as described previously⁵⁶ (FIG. 10a). To identify the most potent and selective DNA-PKcs small molecule inhibitor for enhancing gene targeting efficiency, we performed a screen at the CCR5 locus to knock-in two stop codons using the RNP and AAV6 gene editing platform in PSCs⁵⁷ (FIG. 10b). For this screening, we tested various previously described DNA-PKcs inhibitors, AZD7648^{56,58}, M3814⁵⁹, VX984⁶⁰, KU57788⁶¹, BAY8400⁶² and LTURM⁶³ at three different concentrations (1, 0.1 and 0.01 μM). AZD7648, M3814, VX984 and BAY8400 compounds improved gene targeting efficiency at the concentration of 1 μM. At 0.1 μM, only AZD7648 significantly enhanced the gene targeting efficiency more than 4-fold compared to the untreated control (FIG. 10c, FIG. 15a). Thus, AZD7648 is more potent than the other DNA-PKcs inhibitors for improving gene targeting efficiency. Next, we performed a concentration gradient (1, 0.5, 0.25 and 0.1 μM) and time course (4 h, 8 h, 12 h and 24 h) analysis for gene targeting with AZD7648 treatment. From this analysis, we found that 0.25-0.5 μM and 24 h were the optimal concentration and incubation time respectively for improving gene targeting (FIG. 10d-e, FIG. 15b-c). Notably at the optimal concentration (0.5 and 0.25 μM) for gene targeting, AZD7648 is highly specific for DNA-PKcs inhibition (FIG. 10a).

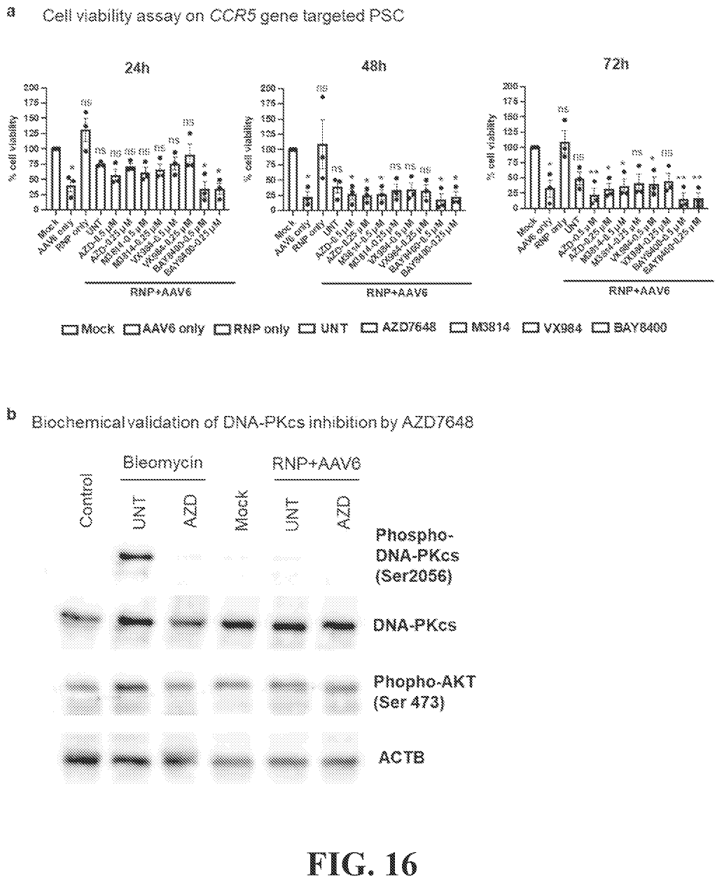

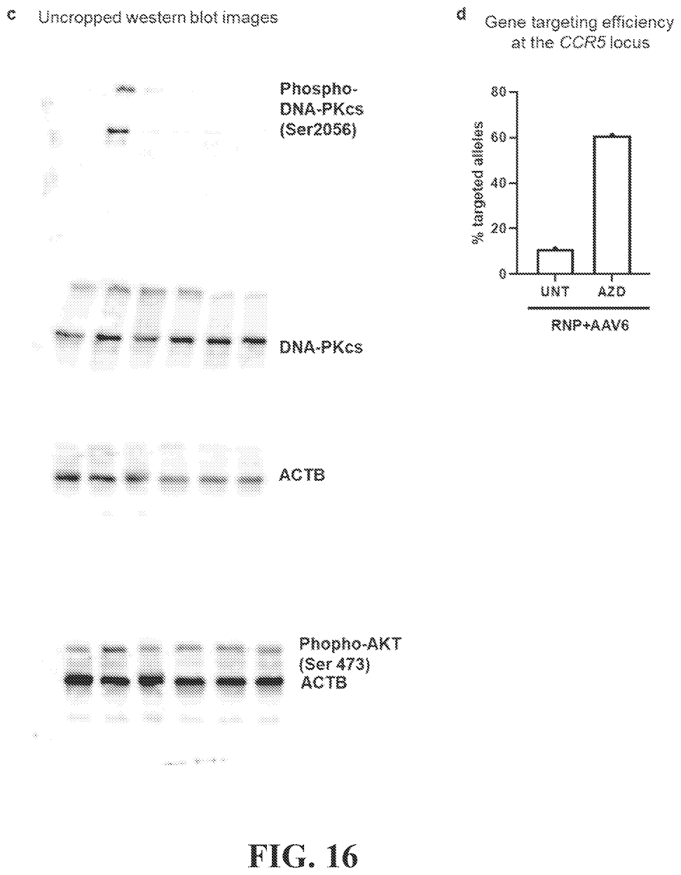

CCR5 is a safe harbor genomic locus that can be used for integration of large gene cassettes with exogenous promoters to safely overexpress genes of interest for therapeutic and research purposes⁶⁴⁻⁶⁷. Thus, we tested whether DNA-PKcs inhibitor treatment can improve the gene targeting efficiency at the CCR5 locus for integration of large gene cassettes. We compared the effect of AZD7648 and other DNA-PKcs inhibitors for gene targeting at the CCR5 locus to knock-in a multi-kb sequence (2.6 kb) (FIG. 10f). We found that AZD7648 significantly enhanced the allelic gene targeting efficiency at 1 and 0.1 μM while M3814 and VX984 were effective only at 1 μM. With AZD7648 treatment, the allelic gene targeting efficiency reached 85%, which is a 2.4-fold increase compared to the untreated control. BAY8400 treatment significantly improved the gene targeting at 0.1 μM (FIG. 10g) but had high toxicity at 1 μM (data not shown). Next, we assessed the cell viability at different time points (24 h, 48 h and 72 h) post CCR5 gene targeting with and without treatment of different DNA-PKcs inhibitors (AZD7648, M3814, VX984 and BAY8400 at 0.5 and 0.25 μM). AZD7648 treatment showed the highest improvement in gene targeting efficiency (FIG. 15d). Cell viability analysis showed that AAV6 transduction alone leads to high levels of toxicity which has been reported in previous studies on AAV transduction in PSCs⁶⁸. Among the gene targeted cells, treatment with DNA-PKcs inhibitors led to slightly higher toxicity at 48 h and 72 h when compared to the untreated cells. BAY8400 was found to be more toxic than other DNA-PKcs inhibitors at all three time points (FIG. 16a). Next, we assessed biochemically whether treatment with AZD7648 effectively inhibits DNA-PKcs activity without affecting the function of kinases belonging to the PI3K family. For DNA-PKcs activity, we assessed the autophosphorylation at Ser2056 site⁵⁶ and for PI3K function, we assessed the phosphorylation of AKT at Ser473 site⁶⁹. We used bleomycin to induce broadscale DNA damage to enhance the detection of DNA-PKcs activity⁷⁰. Western blot analysis showed PSC treated with bleomycin for 2 hours exhibited autophosphorylation of DNA-PKcs (Ser2056) while AZD7648 co-treatment inhibited this autophosphorylation. RNP/AAV6 gene editing did not result in detectable DNA-PKcs autophosphorylation due to minimal activation of the protein since the high-fidelity Cas9 RNP creates just a couple/few breaks in the genome at any given point. Importantly, AZD7648 treatment did not affect the phosphorylation of AKT (Ser473) by PI3K family of kinases (FIG. 16b, c). We also confirmed that AZD7648 treatment improved gene targeting efficiency in PSCs used for this biochemical analysis (FIG. 16d).

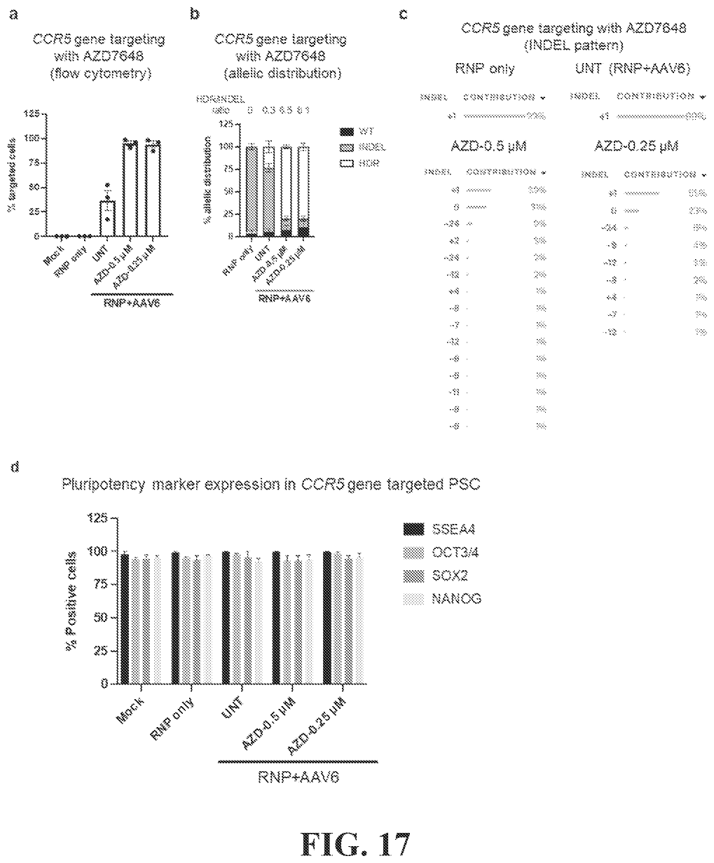

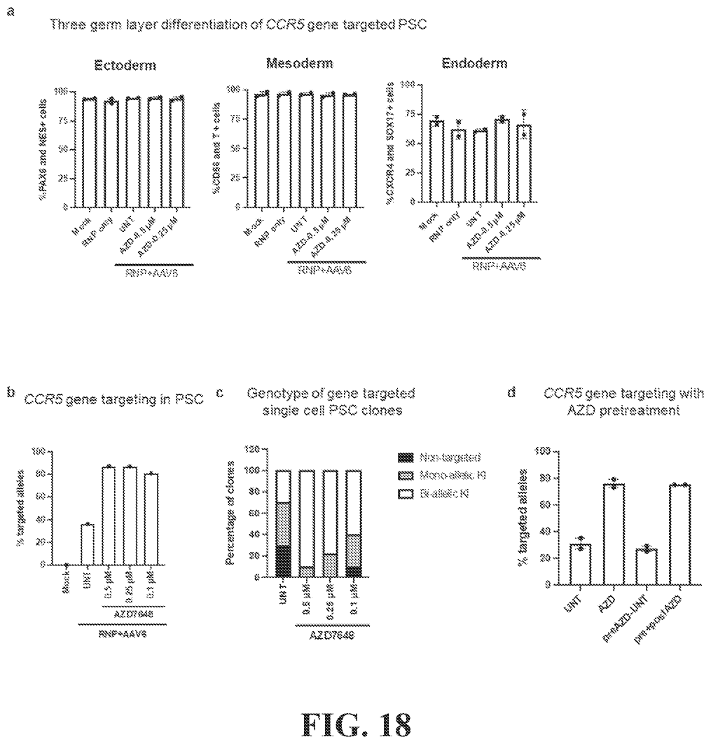

To confirm the maintenance of pluripotency in the gene targeted PSCs, we assessed the expression of pluripotency markers and differentiation into three germ layers following gene targeting at the CCR5 locus. Flow cytometry analysis showed that AZD7648 treatment leads to gene targeting in more than 90% of cells, which was a 2.5-fold increase compared to the untreated control (FIG. 17a). Analysis of the allelic distribution of WT, INDEL and HDR frequencies showed that AZD7648 treatment improved the HDR frequency 3-fold with a corresponding increase in HDR/INDEL ratio from 0.3 to ˜8 (˜24-fold increase) (FIG. 17b). Interestingly, we found that treatment with AZD7648 led to a minor fraction of small to large deletions and a predominant increase in the frequency of WT in the non-targeted alleles while the remaining alleles showed a 1-bp insertion. This 1-bp insertion was the only type of INDEL observed in non-AZD7648 treated genome edited cells (FIG. 17c). We found that the gene targeted PSC with and without the AZD7648 treatment maintained the expression of different pluripotency markers (FIG. 17d). Differentiation of the gene targeted PSCs into three germ layers (ectoderm, mesoderm and endoderm) was also unaffected with AZD7648 treatment, further confirming the maintenance of pluripotency (FIG. 18a).

We performed single cell cloning of PSCs gene targeted at the CCR5 locus with different concentrations of AZD7648 (FIG. 18b) and assessed the genotype of the single cell clones. We found that treatment with 0.5, 0.25 and 0.1 μM of AZD7648 led to derivation of 90%, 80% and 60% clones with bi-allelic knock-in respectively while the untreated cells only had 30% of clones with bi-allelic knock-in (FIG. 18c). This result confirms that AZD7648 treatment leads to an increase in bi-allelic gene targeting. We then tested whether AZD7648 pre-treatment before gene editing would further improve gene targeting efficiency. For this, PSCs were pretreated with AZD7648 for 24 hours before gene targeting and then the cells were either left untreated or with continued AZD7648 treatment post-nucleofection. Allelic gene targeting efficiency was unaffected by pretreatment with AZD7648 with or without post treatment when compared to the corresponding controls. This indicates that pretreatment has no effect and treatment post-editing is essential for improving gene targeting efficiency (FIG. 18d).

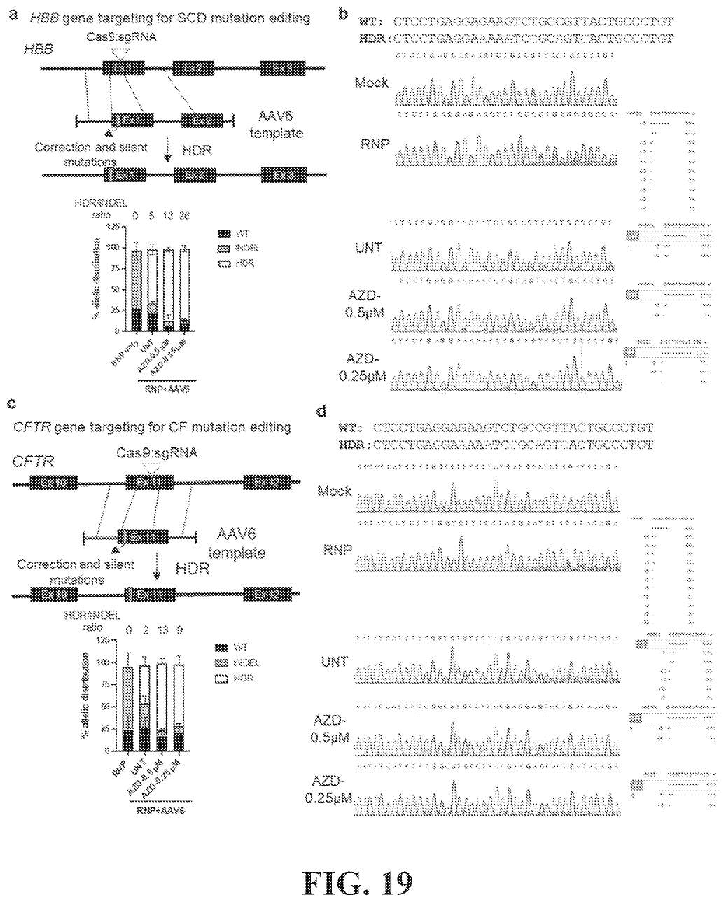

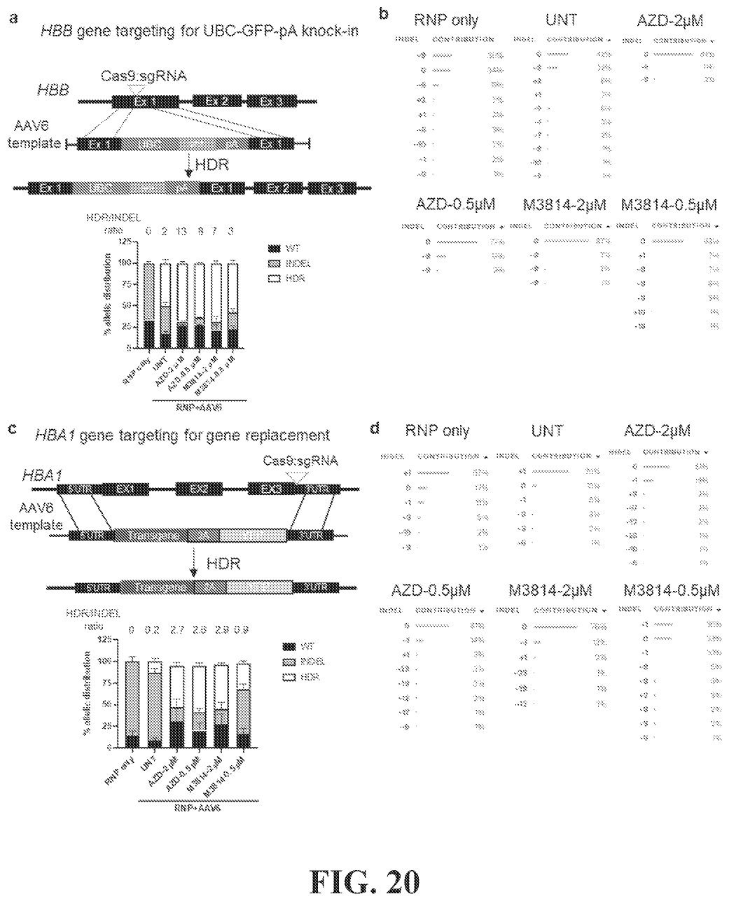

We then tested the effect of AZD7648 treatment for improving gene targeting across different genomic loci in PSC. First, we tested the treatment for editing the sickle cell disease (SCD) and cystic fibrosis (CF) mutations at HBB and CFTR loci respectively using the previously described Cas9 RNP and AAV6 donor reagents in WT PSC⁷¹⁻⁷³ (FIG. 19a-d). At both the concentrations (0.5 and 0.25 μM) tested, AZD7648 treatment improved the gene targeting efficiency at HBB and CFTR loci with a corresponding decrease in the levels of INDELs. Notably, AZD7648 treatment improved the mean ratio of HDR to INDEL from 5 to 26 at the HBB locus and from 2 to 13 at the CFTR locus (FIG. 19a-d). Next, we tested the effect of AZD7648 and M3814 treatment for knock-in of a multi-kb sequence (2.6 kb) at the HBB locus. Although the allelic gene targeting in the untreated cells was quite high at 50%, treatment with AZD7648 or M3814 improved the gene targeting by about 1.4-fold at the higher concentration (2 μM) and only AZD7648 showed a significant improvement at the lower concentration (0.5 μM) (FIG. 20a). Non-gene targeted alleles in the untreated cells showed 9-bp deletion as the major INDEL while there was also a small fraction of insertions and other deletions, resembling the pattern in the RNP only control. AZD7648 treatment led to an increase in the frequency of WT alleles and the remaining alleles had a small fraction of the 9-bp deletion (FIG. 20b). We then assessed whether gene replacement efficiency can be improved with the AZD7648 treatment at the HBA1 locus using the previously described gene editing strategy⁷⁴. AZD7648 treatment improved the HBA1 gene replacement efficiency by around 4-fold at both high and low concentrations (2 and 0.5 μM) while M3814 was effective only at the higher concentration (2 μM) (FIG. 20c). Non-gene targeted alleles in the untreated cells showed 1-bp insertion as the major INDEL while there was also a small fraction of alleles with deletions, resembling the RNP only control. AZD7648 treatment led to an increase in the frequency of WT alleles, a small fraction of alleles with deletions and little to no alleles with the 1-bp insertion (FIG. 20d).

In conclusion, these results confirm that AZD7648 is the current most potent DNA-PKcs inhibitor for enhancing gene targeting efficiency across different genomic loci in PSCs without affecting the pluripotency.

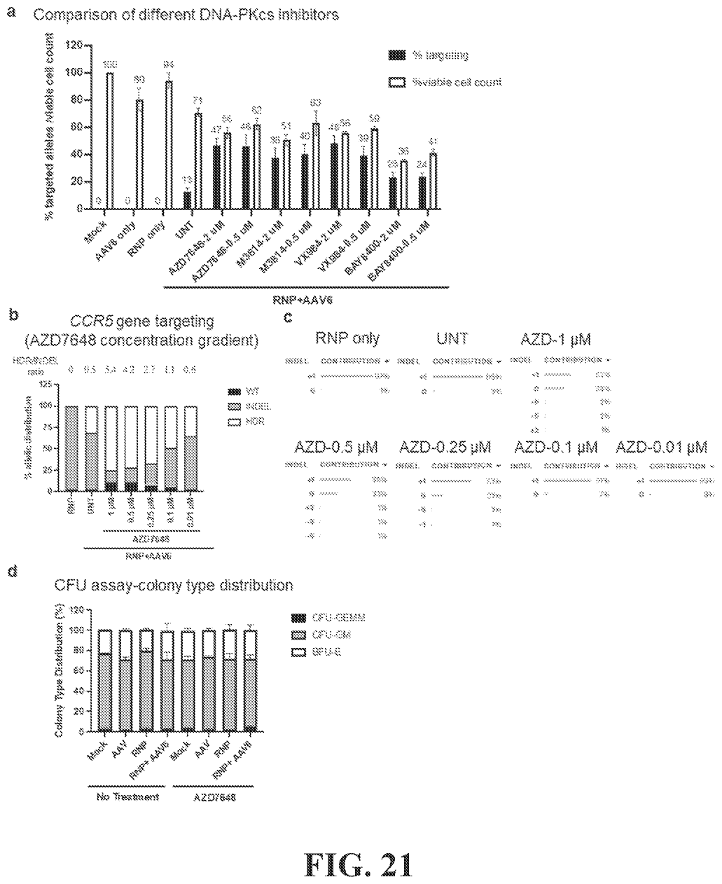

AZD7648 treatment improves gene targeting across different loci in HSPCs. We compared the effect of various DNA-PKcs inhibitors (AZD7648, M3814, VX984 and BAY8400) at two different concentrations (2 μM and 0.5 μM) for improving gene targeting at the CCR5 locus in primary human CD34+ HSPCs. Allelic gene targeting efficiency and viable cell count indicated that treatment with 0.5 μM was less toxic than 2 μM with similar gene targeting efficiency for all compounds. At 0.5 μM, AZD7648 showed the highest improvement in gene targeting efficiency when compared to other compounds (FIG. 21a). We determined the optimal concentration of AZD7648 treatment with a concentration gradient analysis (1-0.01 μM) for gene targeting at the CCR5 locus. We found that the gene targeting efficiency remained high between 0.25-0.5 μM, which is similar to the results from PSCs, and we used 0.5 μM for all subsequent gene targeting experiments in HSPCs (FIG. 21b). In the non-gene targeted alleles, we found that AZD7648 treatment at 1, 0.5 and 0.25 μM concentrations led to an increase in the frequency of WT alleles and a decrease in the frequency of alleles with 1-bp insertion when compared to the untreated cells (FIG. 21c).

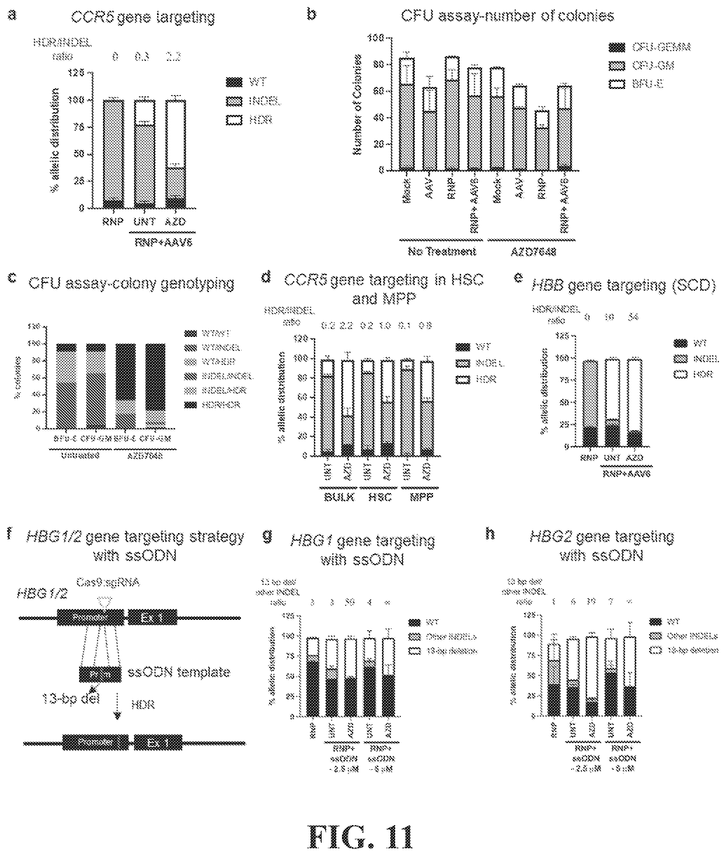

We then tested the effect of AZD7648 on gene targeting across different therapeutically relevant genomic loci in HSPCs. With AZD7648 treatment at the optimal 0.5 μM concentration, allelic gene targeting efficiency improved by around 3-fold with a concomitant 7-fold increase in HDR to INDEL ratio at the CCR5 safe harbor locus for knock-in of a multi-kb sequence (2.6 kb) (FIG. 11a). To confirm that gene targeting with AZD7648 does not affect the ability of HSPCs to differentiate into different hematopoietic lineages, we performed a colony forming units (CFU) assay in vitro. For this, we used CCR5 gene targeted HSPCs (RNP+AAV6) with and without AZD7648 treatment. As controls, we used mock, AAV6 only and RNP only treated cells with and without AZD7648 treatment and assessed the colony formation. As has been previously described, the RNP/AAV6 system does cause a decrease in the total number of colonies without a change in distribution⁷⁴⁻⁷⁷ but AZD7648 treatment did not cause further decrease in total colony number nor a change in the colony type distribution (FIG. 11b, FIG. 21d). We determined the genotype of the gene targeted HSPC-derived BFU-E and CFU-GM colonies from the CFU assay and found that AZD7648 treatment increased the frequency of colonies with bi-allelic HDR knock-in by more than 7-fold in both colony types (9% to 67% increase in BFU-E and 9% to 78% increase in CFU-GM) (FIG. 11c). Thus, AZD7648 treatment increases the frequency of bi-allelic HDR knock-in without affecting the differentiation potential of HSPCs.

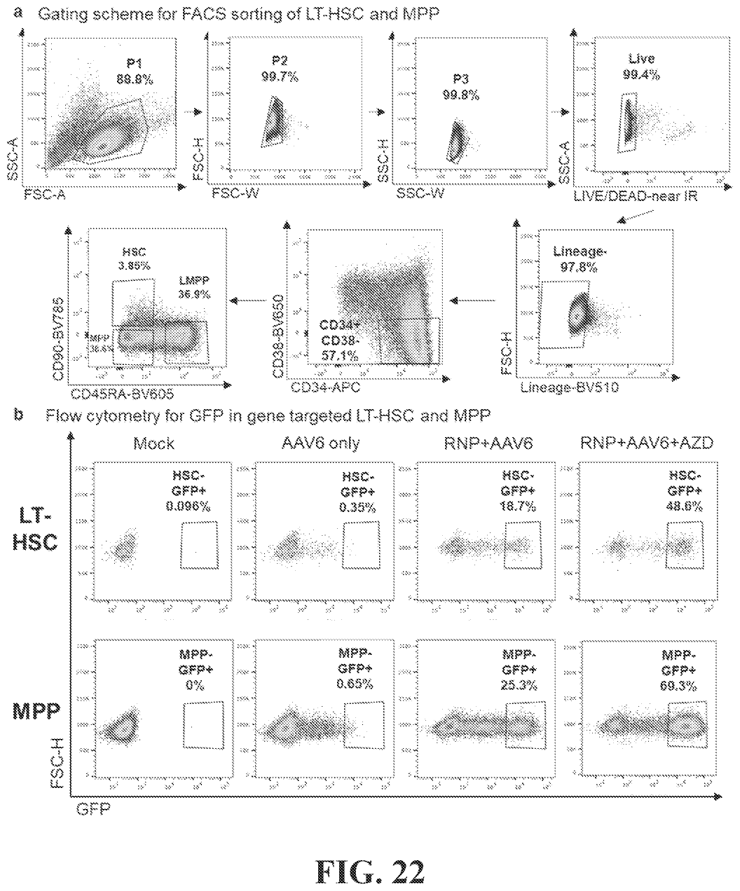

We assessed whether AZD7648 treatment improved gene targeting in long term hematopoietic stem cells (LT-HSC) and multi-potent progenitors (MPP). For this, we gene targeted CD34+ HSPCs at the CCR5 locus with and without AZD7648 treatment and then FACS sorted the LT-HSC and MPP populations (FIG. 22a). AZD7648 treatment improved the frequency of gene targeted cells in LT-HSC and MPP populations by more than 2.5-fold as assessed by flow cytometry for GFP (FIG. 22b). Allelic gene targeting efficiency was improved by almost 4-fold with AZD7648 treatment in LT-HSC and MPP populations with a concomitant increase in HDR to INDEL ratio (FIG. 11d). Thus, AZD7648 treatment improves gene targeting in phenotypic long term hematopoietic stem cells which are essential for achieving sustained clinical benefit in potential therapeutic applications.

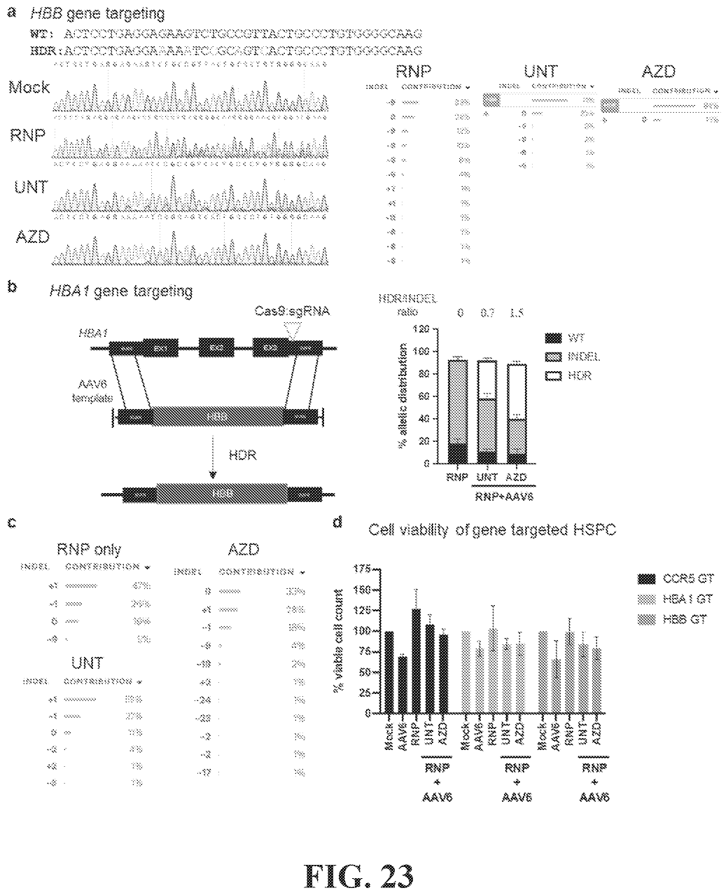

We tested AZD7648 treatment for editing the SCD mutation at the HBB locus (FIG. 19A, upper panel)^{71,72}. HBB gene targeting efficiency was improved with the compound treatment to nearly INDEL-free levels with a 5-fold (10 to 54) increase in the HDR to INDEL ratio (FIG. 11e, FIG. 23a). Next, we tested the AZD7648 treatment for gene replacement at the HBA1 locus with HBB gene sequence, a previously reported therapeutic gene editing strategy for β-thalassemia⁷⁴. We found that this gene replacement efficiency improved by about 1.4-fold with AZD7648 treatment with a 2-fold increase in HDR to INDEL ratio (FIG. 23b). The improvement in gene targeting was only modest possibly because of the use of split homology arms limiting the improvement in the frequency of HDR-based targeted integration. In the non-gene targeted alleles, we found that AZD7648 treatment led to an increase in the frequency of WT alleles and a decrease in the frequency of alleles with 1-bp insertion when compared to the untreated cells (same as observed at the CCR5 locus, described above) (FIG. 23c). Cell viability was assessed in HSPCs post-gene targeting at the CCR5, HBA1 and HBB loci. Similar to PSCs, we found some toxicity with transduction of AAV6 donors alone in HSPCs as described previously⁷⁸. But importantly, gene targeting with AZD7648 treatment resulted in only a small drop in viable cell count at 72 h post-gene targeting at all the three loci when compared to the mock cells (FIG. 23d).

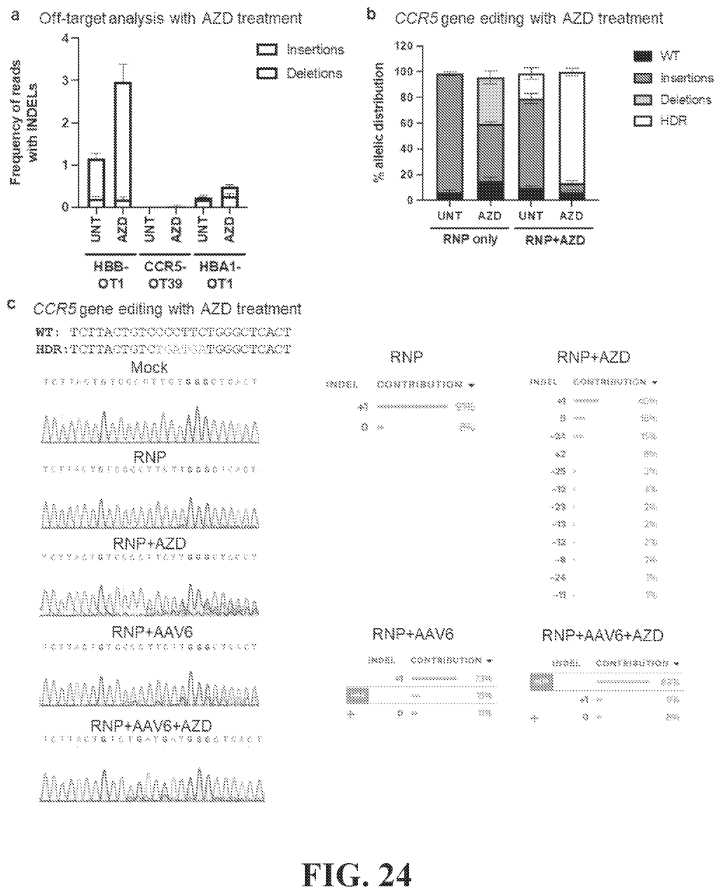

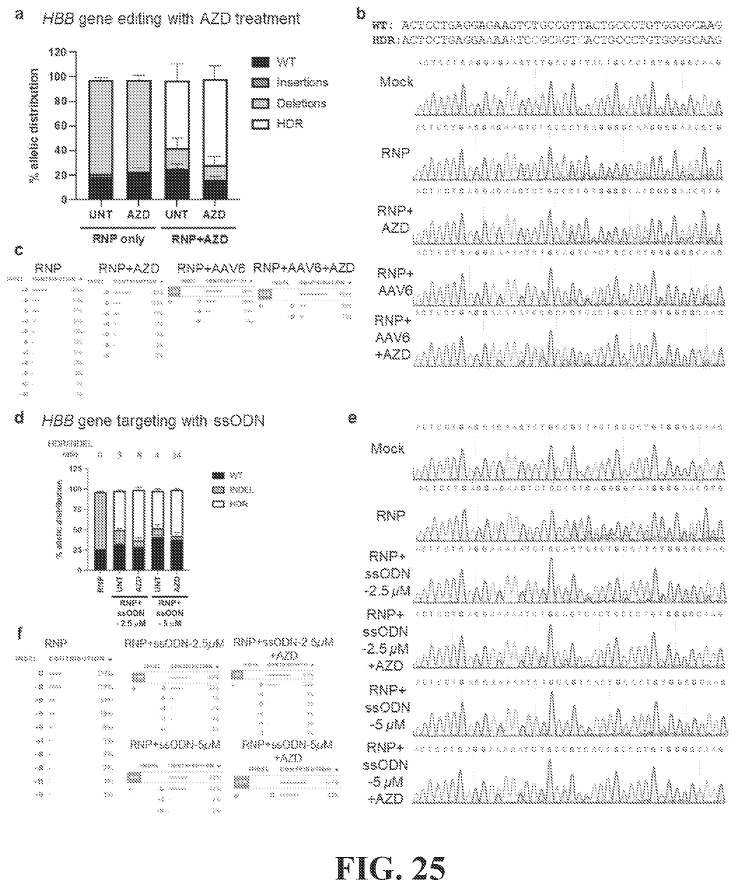

We then tested whether gene targeting with AZD7648 treatment affected the off-target activity of Cas9 RNP gene editing. For this, we assessed the previously characterized top off-target site for CCR5 (OT39)⁶⁶, HBB (OT1)⁷² and HBA1 (OT1)74 gRNAs in the gene targeted HSPCs using next generation sequencing (NGS). At the OT1 site for HBB gRNA, we found 1% reads had INDELs in the untreated cells while AZD7648 treated cells had about 3% of reads with INDELs. At the OT39 site for CCR5 gRNA, AZD7648 treated cells showed 0.03% of reads with INDELs while the frequency of reads with INDELs was below the detection limit in the untreated cells. At the OT1 site for the HBA1 gRNA, AZD7648 treated cells had INDELs in around 0.5% reads while the untreated cells had INDELs in 0.24% reads. Interestingly, the small increase in off-target activity observed with AZD7648 treatment was due to an increase in the frequency of deletion INDELs at all three assessed off-target sites and this is likely due to the utilization of the MMEJ pathway⁴⁰ (FIG. 24a). Blocking of specific off-target breaks is possible, however, if there were biologically important reasons to do so^{79, 80}. Next, we assessed whether on-target activity of Cas9 RNP would be affected by AZD7648 treatment in the absence of AAV6 donor template. For this, we used CCR5 and HBB gRNAs and assessed the pattern of INDEL following AZD7648 treatment with and without AAV6 donor template. At the CCR5 locus, we found an increase in the frequency of WT and deletion INDELs with AZD7648 treatment in the RNP only cells. With the addition of AAV6 donor template to introduce two stop codons, we found that AZD7648 treatment enhances the HDR efficiency without affecting the pattern of INDELs in the non-gene targeted alleles (FIG. 24b-c). At the HBB locus, we found that AZD7648 treatment in the RNP only cells eliminated the small fraction of alleles with insertion INDELs and increased the frequency of WT alleles. Addition of AAV6 donor template with AZD7648 treatment led to an increase in HDR without affecting the pattern of the INDELs in the non-targeted alleles (FIG. 25a-c). Thus, gene editing with AZD treatment increases the frequency alleles with WT sequence and deletion INDELs without the HDR donor template.

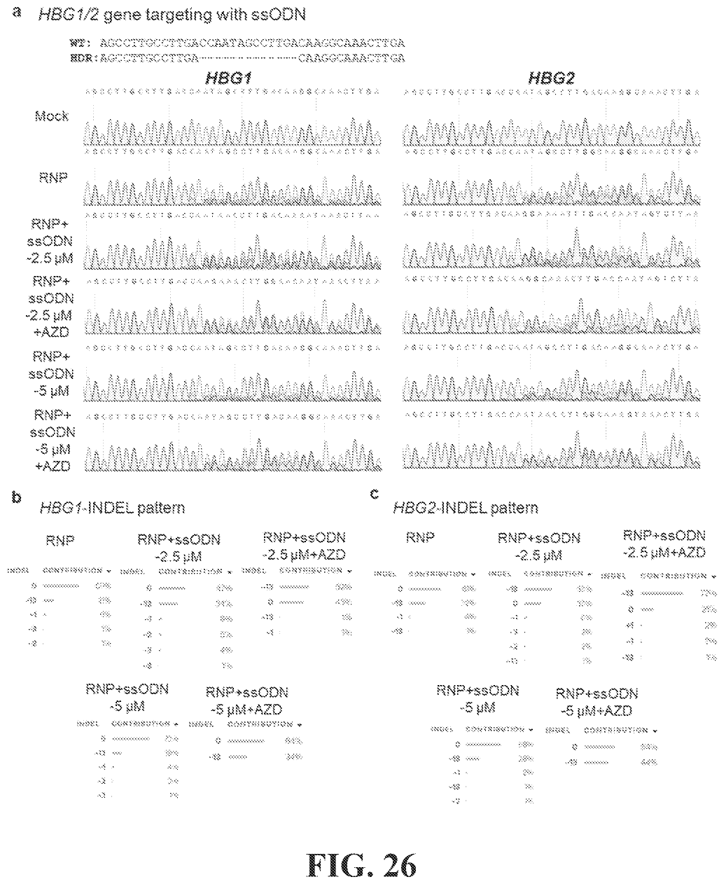

To confirm whether AZD7648 treatment is also relevant for gene targeting using ssODN based-donor template delivery, we tested SCD mutation editing with Cas9 RNP and ssODN donor in WT HSPCs⁸¹. With the lower concentration of ssODN donor tested (2.5 μM), AZD7648 treatment improved the gene targeting frequency from 49% to 65% with a corresponding 2.7-fold increase in the HDR to INDEL ratio. At the higher concentration of ssODN, the gene targeting frequency was improved to a lesser extent with AZD7648 treatment but there was a higher increase in HDR to INDEL ratio due to an increase in the frequency of WT alleles (FIG. 25d-f). Next, we further tested the applicability of AZD7648 treatment with ssODN donor for gene targeting at HBG1/2 loci to activate the fetal hemoglobin expression (a therapeutic strategy for β-hemoglobinopathies)¹². For this, we tested the feasibility of using an ssODN donor to introduce a naturally occurring 13-bp deletion mutation in the promoters of HBG1/2, which is associated with a benign condition called hereditary persistence of fetal hemoglobin (HPFH)^{83, 84} (FIG. 11f). We used a previously reported gRNA near the target site which, when delivered with Cas9 as RNP in HSPCs led to a low frequency of the precise 13-bp deletion. This likely occurred through MMEJ repair of the DSB due to the presence of a 6-bp microhomology regions around the cut site⁸⁵. In accordance with the previous study, we observed around 20% frequency of the 13-bp deletion in both HBG1/2 promoters with Cas9 RNP delivery. Inclusion of the ssODN donor with Cas9 RNP (untreated, UNT) resulted in a 2 to 2.5-fold increase in the frequency of the 13-bp deletion due to an HDR-based deletion. AZD7648 treatment improved the 13-bp deletion frequency further by about 1.3 to 1.5-fold when compared to the untreated (UNT) cells with a concomitant increase in the ratio of the 13-bp deletion to other INDELs by several fold (FIG. 11g-h, FIG. 26a-c). Thus, these results confirm the applicability of AZD7648 treatment for improving gene targeting across different genomic loci with either AAV6 or ssODN-based HDR donors in HSPCs.

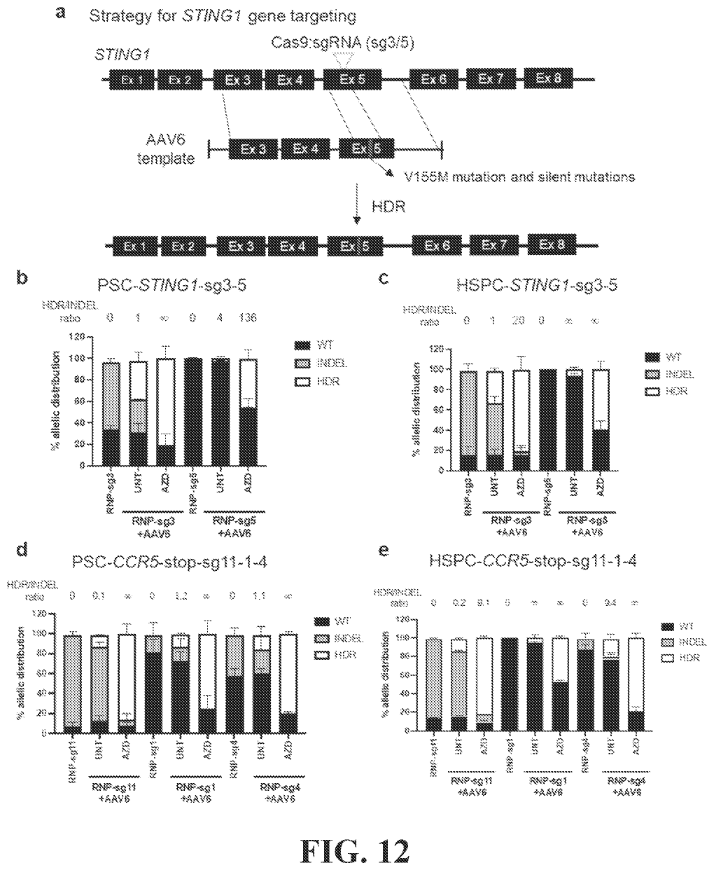

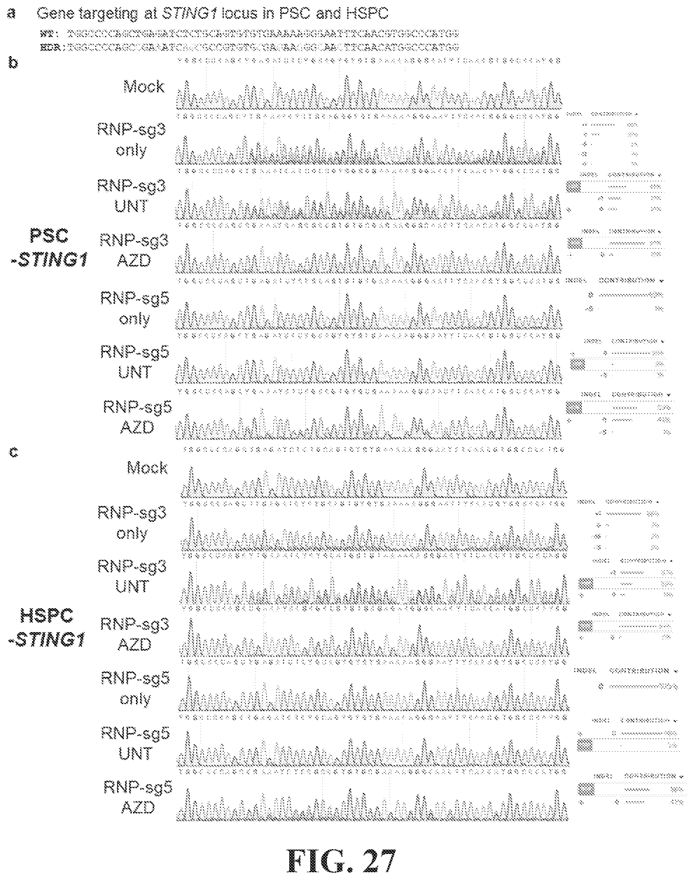

AZD7648 treatment improves gene targeting with seemingly inactive and low activity gRNAs. We tested whether AZD7648 treatment can improve the gene targeting efficiency while using a seemingly inactive gRNA which creates very low levels of INDELs following Cas9-gRNA RNP delivery. For this, we tested gene targeting at the STING1 locus to introduce a point mutation (V155M) associated with an autoinflammatory disease called STING-associated vasculopathy with onset in infancy (SAVI)⁸⁶ (FIG. 12a). Following a gRNA screening near the mutation site in exon 5 of the STING1 gene, we identified a seemingly inactive gRNA (sg5) which generates only 1% INDELs and a highly active gRNA (sg3) which generates around 70% INDELs following RNP delivery. Gene targeting with the seemingly inactive gRNA improved from about 1% to around 50% with AZD7648 treatment with no INDELs in PSCs. With the active gRNA, HDR frequency was improved by 2.3-fold with no INDELs following AZD7648 treatment in PSCs (FIG. 12b, FIG. 27a-b). To test the feasibility of building a primary cell SAVI model, we next performed the STING1 gene targeting in human CD34+ HSPCs. With the seemingly inactive gRNA, STING1 gene targeting efficiency in HSPCs improved by almost 10-fold (from 6% to 59%) with no INDELs following AZD7648 treatment. Gene targeting with the active gRNA improved gene targeting efficiency by 2.5-fold in HSPCs with a corresponding increase in HDR to INDEL ratio from 1 to 20 (FIG. 12c, FIG. 27c).

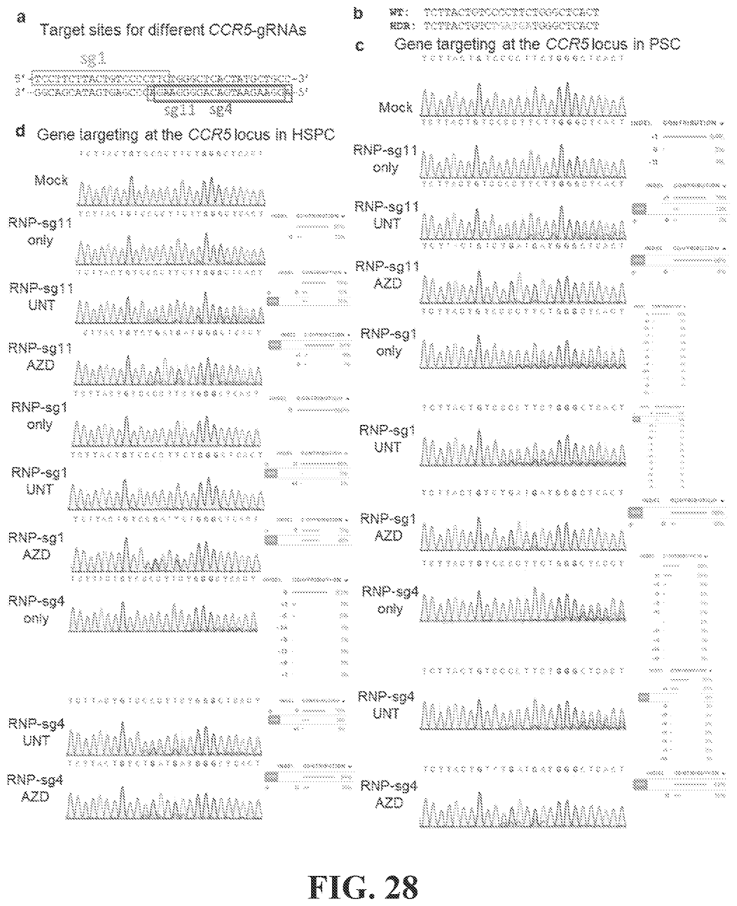

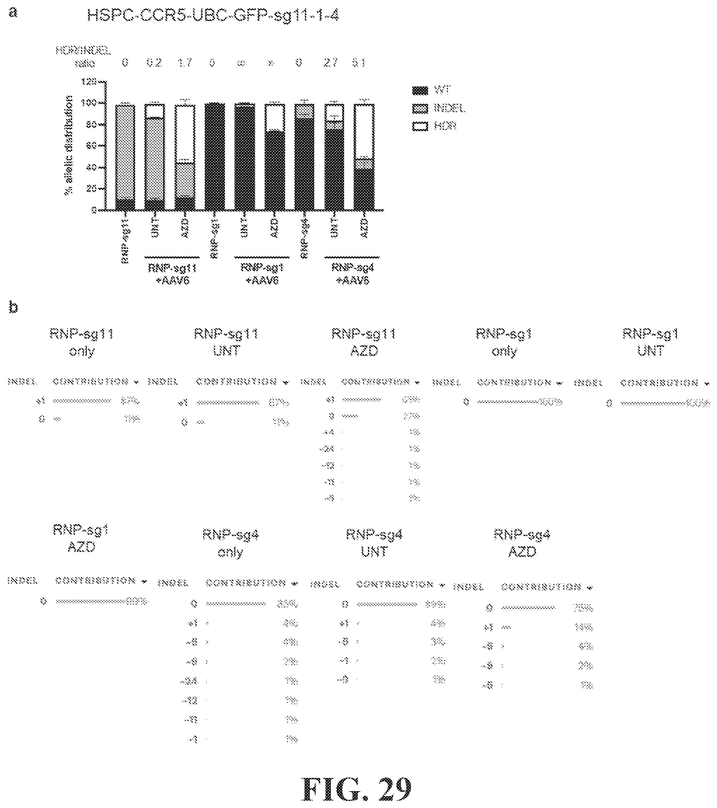

Next, we assessed gene targeting at the CCR5 locus using low activity gRNAs, sg1 and 4 with AZD7648 treatment in PSCs and HSPCs (FIG. 28a). sg1 and 4 RNP only gene editing resulted in 17.5% and 40% INDELs respectively in PSC while editing with the high activity gRNA, sg11 resulted in 90% INDELs. Gene targeting with AZD7648 treatment resulted in INDEL-free 75% and 80% allelic gene targeting efficiency with sg1 and sg4 gRNAs respectively for the introduction of two stop codons while the HDR efficiency was less than 20% in the non-AZD7648 treated cells (FIG. 12d, FIG. 28b-c). In HSPCs, sg1 and sg4 RNP only gene editing resulted in 0% and 11.5% INDELs respectively while sg11 RNP showed 85% INDELs. Gene targeting with AZD7648 treatment yielded INDEL-free HDR at an efficiency of 47.5% and 79% respectively, which is 4- to 5-fold higher than that of untreated controls for introduction of two stop codons in HSPCs (FIG. 12e, FIG. 28d). To confirm whether this finding is applicable for insertion of large gene cassettes, we tested gene targeting at the CCR5 locus for knock-in of multi-kb sequence (2.6 kb) in HSPCs. With sg1, AZD7648 treatment resulted in INDEL-free HDR efficiency of 25% while the untreated cells only had 3.5% HDR. With sg4, AZD7648 treatment yielded a HDR efficiency of 50%, a 5-fold increase when compared to the untreated cells. Gene targeting using high activity sg11 gRNA with AZD7648 treatment resulted in 54% HDR efficiency (FIG. 29a). In the non-gene targeted alleles, AZD7648 treatment resulted in a small fraction of deletion INDELs which were absent in untreated cells for sg11 and sg4. For sg1, all the non-gene targeted alleles were found to be WT alleles with and without AZD7648 treatment (FIG. 29b).

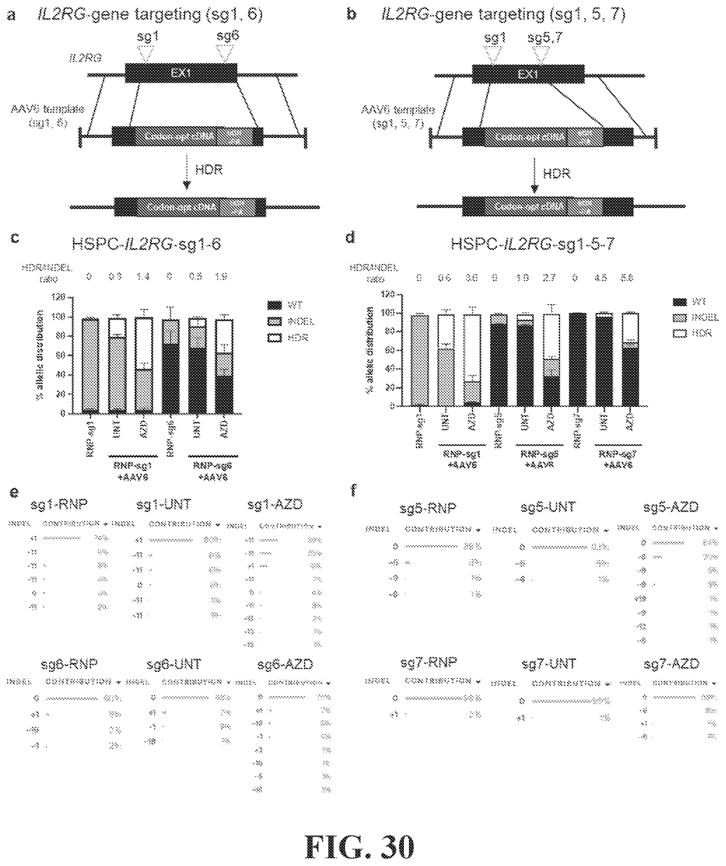

We then tested whether this finding would be applicable to gene targeting at exon 1 of IL2RG locus using previously characterized high (sg1) and low activity gRNAs (sg5, 6 and 7) in HSPCs⁹⁹. For this gene targeting, we designed two different AAV6 HDR donor template vectors with split homology arms (one vector for sg1 and 6, the other for sg1, 5 and 7, FIG. 30a-b). sg1 RNP only editing resulted in 94% INDELs while editing with sg6 yielded only 26% INDELs. Gene targeting with AZD7648 treatment yielded a HDR efficiency of around 35% with sg6 gRNA which was more than 4-fold higher than that of untreated cells. With sg1, AZD7648 treatment resulted in gene targeting efficiency of 53%, a 2.8-fold increase when compared to non-AZD7648 treated cells. (FIG. 30c). sg5 and 7 RNP only editing resulted in low INDEL frequency of 11% and 2% respectively in HSPCs. With AZD7648 treatment, sg5 and 7 yielded gene targeting efficiency of around 49% and 31% respectively in HSPCs and this was more than a 6-fold increase when compared to the HDR efficiency in untreated cells (FIG. 30d). In the non-gene targeted alleles, AZD7648 treatment increased the frequency of deletion INDELs with all four sgRNAs at IL2RG locus and interestingly reduced the frequency of WT alleles with the three low activity gRNAs (FIG. 30e-f). Importantly, at all three loci (STING1, CCR5 and IL2RG), gene targeting with seemingly inactive and low activity gRNAs yielded higher HDR efficiency with AZD7648 treatment when compared to the untreated cells gene targeted with the corresponding high activity gRNAs.

Thus, AZD7648 treatment can improve gene targeting dramatically even with seemingly inactive and low activity gRNAs across different genomic loci and can achieve INDEL-free HDR in human PSCs and HSPCs.

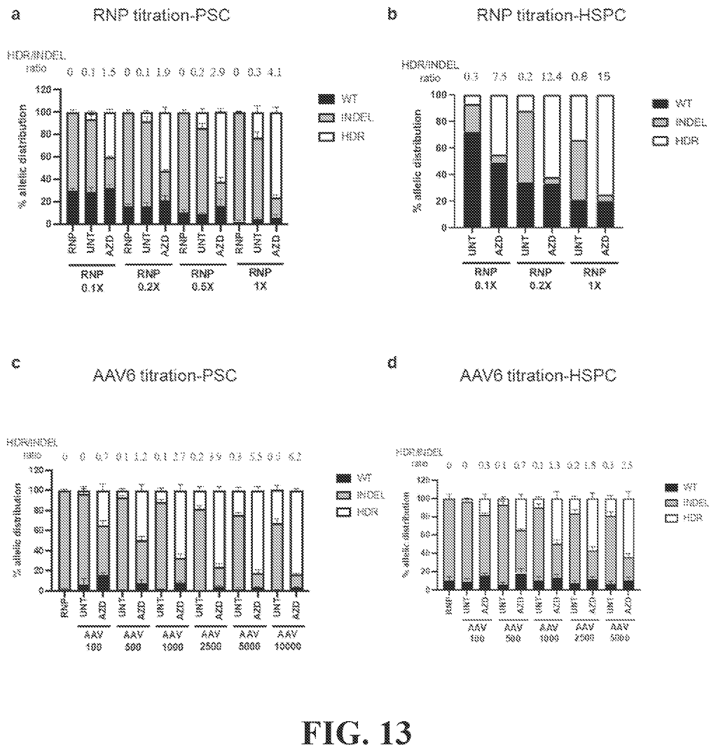

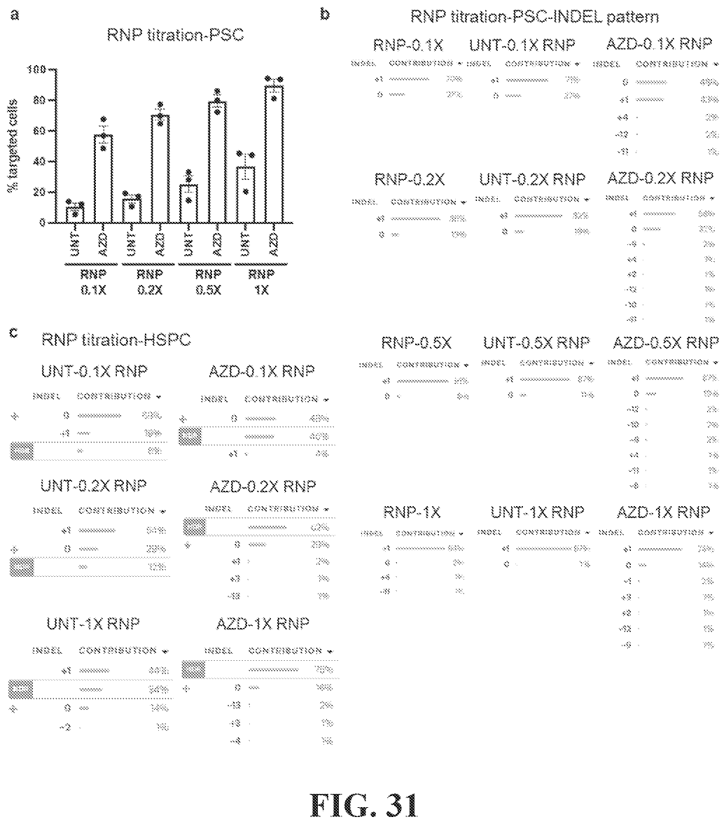

Maintenance of high efficiency gene targeting with lower amounts of RNP and AAV6. We tested whether AZD7648 treatment allows for titrating down the amounts of Cas9 RNP and AAV6 donor without affecting the gene targeting efficiency. For RNP titration in PSCs, we tested reducing the Cas9 RNP by 2-, 5- and 10-fold for gene targeting at the CCR5 locus. Titrating down the amount of RNP in untreated cells resulted in a RNP dose-dependent decrease in gene targeting efficiency. With the AZD7648 treatment, gene targeting efficiency was consistently higher at different doses of RNP. Notably, allelic gene targeting efficiency and HDR to INDEL ratio with AZD7648 treatment at 10-fold lower dose of RNP (0.1×) was about 2- and 5-fold higher than the untreated cells with full dose of RNP (1×) (FIG. 13a). We confirmed these results by measuring the frequency of gene targeted cells using flow cytometry which showed a similar pattern (FIG. 31a). AZD7648 treatment increased the frequency of WT alleles and resulted in a small fraction of deletion INDELs in the non-gene targeted alleles and this pattern was consistent with different amounts of RNP (FIG. 31b). We then tested the effect of RNP titration in human HSPCs for gene targeting at the STING1 locus. Similar to PSCs, gene targeting efficiency and HDR to INDEL ratio with lower doses of RNP were consistently higher with AZD7648 treatment in HSPCs (FIG. 13b, FIG. 31c).

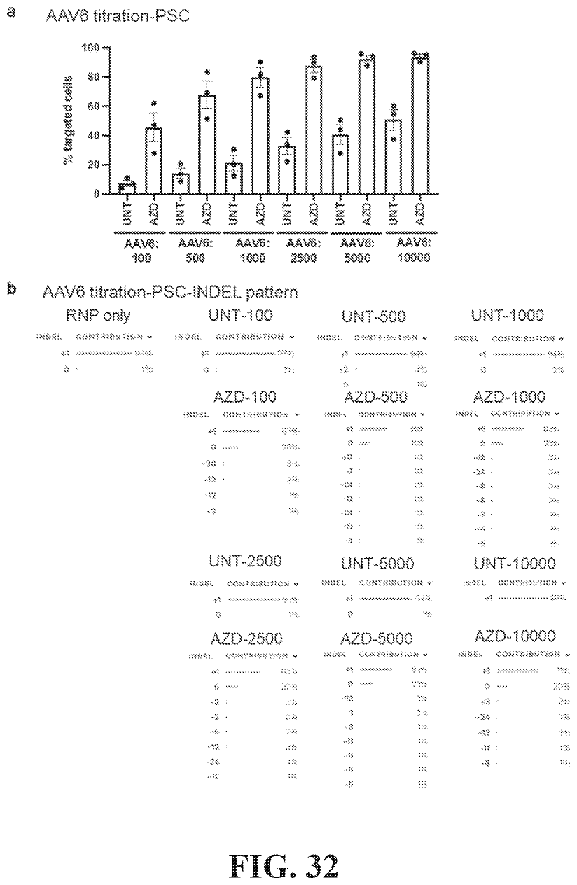

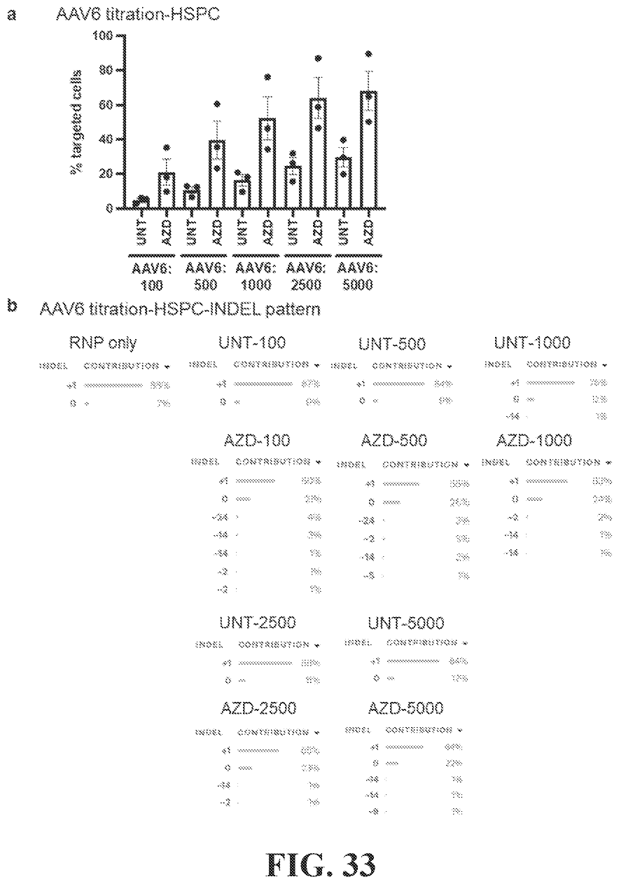

We tested the effect of titrating down the AAV6 donor multiplicity of infection (MOI) in human PSCs and HSPCs (100 to 10,000 for PSCs and 100 to 5,000 for HSPCs) for gene targeting at the CCR5 locus. Allelic gene targeting efficiency and HDR to INDEL ratio with AZD7648 treatment at an AAV6-MOI of 500 were higher than that of untreated cells at the highest tested MOI of 10,000 for PSCs and 5,000 for HSPCs (FIG. 13c-d). Flow cytometry analysis for frequency of gene targeted cells confirmed these findings in PSCs (FIG. 32a). With AZD7648 treatment, the non-gene targeted alleles showed an increase in WT allele frequency and a small fraction of deletion INDEL alleles which were absent in the untreated cells (FIG. 32b). In HSPCs, frequency of gene targeted cells at an AAV6-MOI of 500 with AZD7648 treatment was higher than that of untreated cells at an AAV6-MOI of 5000 (FIG. 33a). Similar to the results in PSC, AZD7648 treatment resulted in an increase in the frequency of WT alleles and a small fraction of alleles with deletion INDELs in the non-gene targeted alleles (FIG. 33b).

Thus, these results show that AZD7648 treatment allows for reducing the amounts of RNP and AAV6 with maintenance of high gene targeting efficiencies.

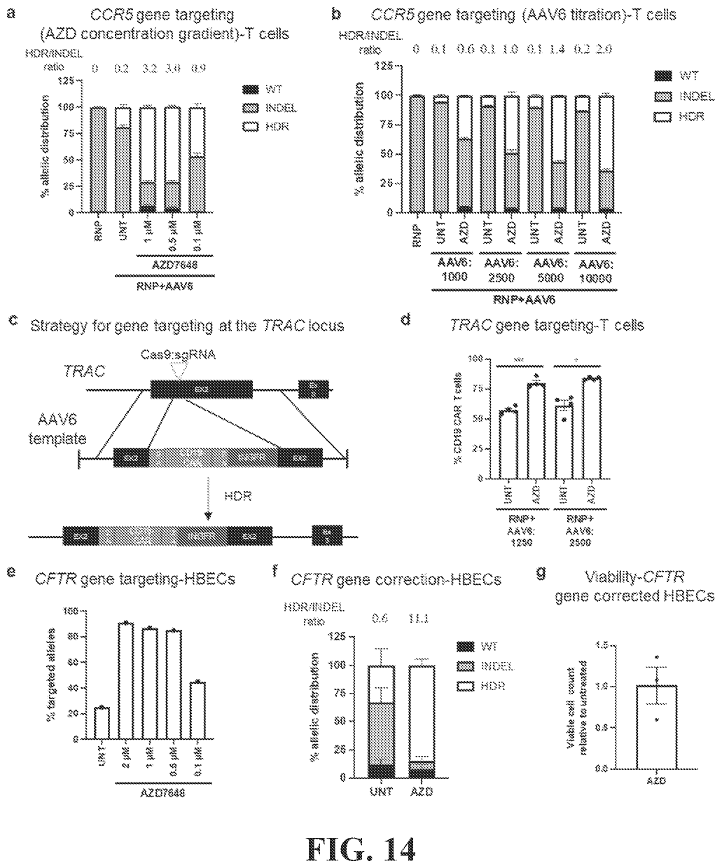

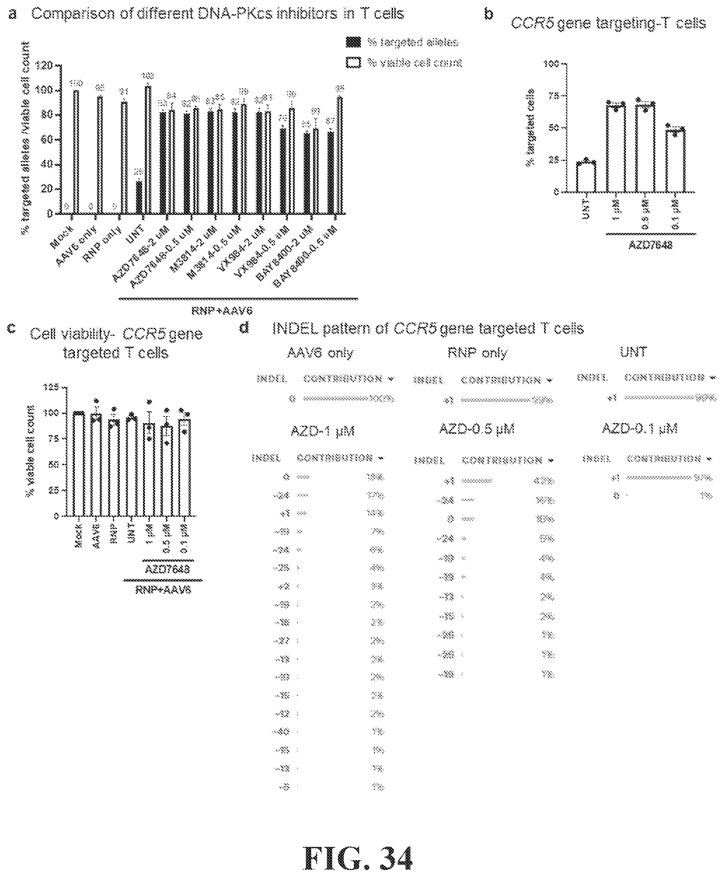

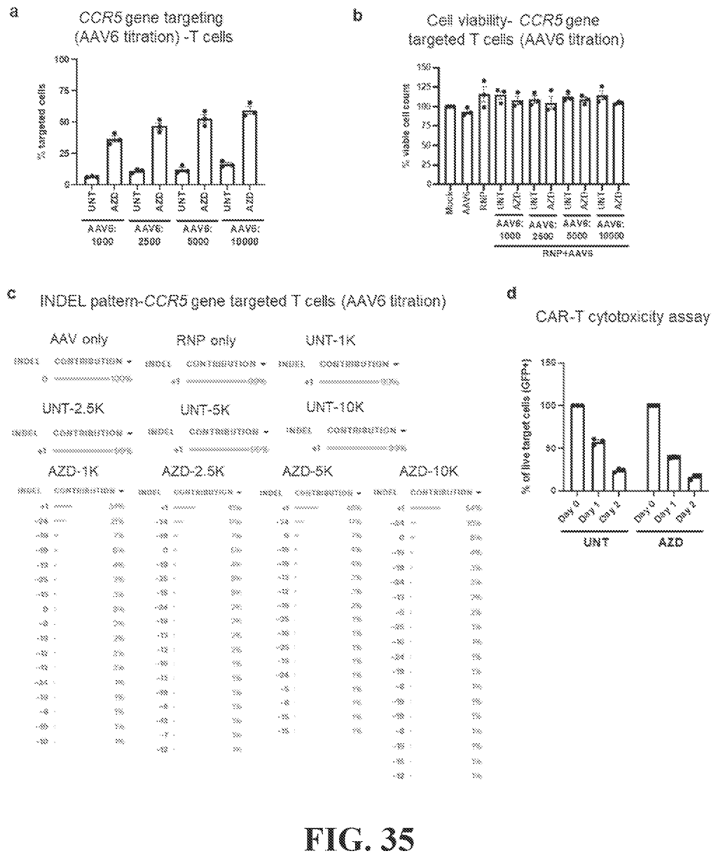

AZD7648 treatment improves gene targeting in T and B cells. We then compared the effect of various DNA-PKcs inhibitors (AZD7648, M3814, VX984 and BAY8400) at two different concentrations (2 μM and 0.5 μM) for improving gene targeting at the CCR5 locus in primary human T cells. Allelic gene targeting efficiency and viability count indicated that treatment 0.5 μM was less toxic than 2 μM with similar gene targeting efficiency for all compounds. At 0.5 μM, AZD7648 and M3814 showed the highest improvement in gene targeting efficiency when compared to the other two compounds (FIG. 34a). Next, we further tested and optimized the AZD7648 treatment for gene targeting in T cells. Following a concentration gradient analysis, we found that 0.5 μM was the optimal concentration of the compound for gene targeting at the CCR5 safe harbor locus in T cells, similar to the findings in other cell types (FIG. 14a, FIG. 34b). Importantly, we observed a minimal drop in viable cell count at 72 h post-gene targeting with different concentrations of AZD7648 when compared to mock and untreated controls (FIG. 34c). In the non-gene targeted alleles, AZD7648 treatment (1 and 0.5 μM) increased the frequency of WT alleles with a concomitant decrease in the alleles with 1-bp insertion INDEL and there was a small fraction of alleles with deletion INDELs which was absent in untreated cells. At 0.1 μM, AZD7648 treatment did not affect the INDEL pattern in the non-targeted alleles. (FIG. 34d). Next, we tested the effect of compound treatment when titrating down the AAV6 donor amounts for gene targeting at the CCR5 locus. At the different AAV6 MOIs tested (1,000-10,000), we found that allelic gene targeting efficiency with AZD7648 was consistently higher with a corresponding increase in HDR to INDEL ratio. Notably, the HDR to INDEL ratio at an MOI of 1000 with AZD7648 treatment was nearly 3-fold higher than the untreated control at an MOI of 10,000 (FIG. 14b). We also found that the frequency of gene targeted cells remained consistently high with AZD7648 at different AAV6 MOIs with very little drop in viable cell count (FIG. 35a-b). In the non-gene targeted alleles, AZD7648 treatment induced a small fraction of deletion INDELs which was absent in untreated cells and this pattern was largely unaffected by the amount of AAV6 (FIG. 35c). We then tested the effect of AZD7648 treatment in T cells for the knock-in of a chimeric antigen receptor (CAR), which is therapeutically relevant for cancer immunotherapies. For this, we assessed the CD19-CAR knock-in at the TRAC gene locus in T cells using a previously reported gene editing strategy⁸⁷ (FIG. 14c). At two different AAV6 MOIs tested (1250 and 2500), we found that gene targeting with AZD7648 treatment resulted in a 1.4-fold increase in the frequency of CD19-CAR+ T cells compared to the untreated controls (FIG. 14d). To confirm that gene targeting with AZD7648 treatment does not affect the T cell function, we assessed the cytotoxicity activity of the CD19-CAR+ T cells against a CD19+ Nalm6 leukemia cell line expressing GFP. CD19-CAR+ T cells and Nalm6 cells were co-cultured at a 1:1 ratio and the cytotoxic activity of T cells was assessed by flow cytometry for GFP at 24 h and 48 h. CD19-CAR+ T cells generated with and without AZD7648 treatment showed similar cytotoxic activity confirming that gene targeting with AZD7648 does not affect the T cell function (FIG. 35d). Thus, AZD7648 treatment can enhance the efficiency of therapeutically relevant gene targeting in T cells without affecting the function.

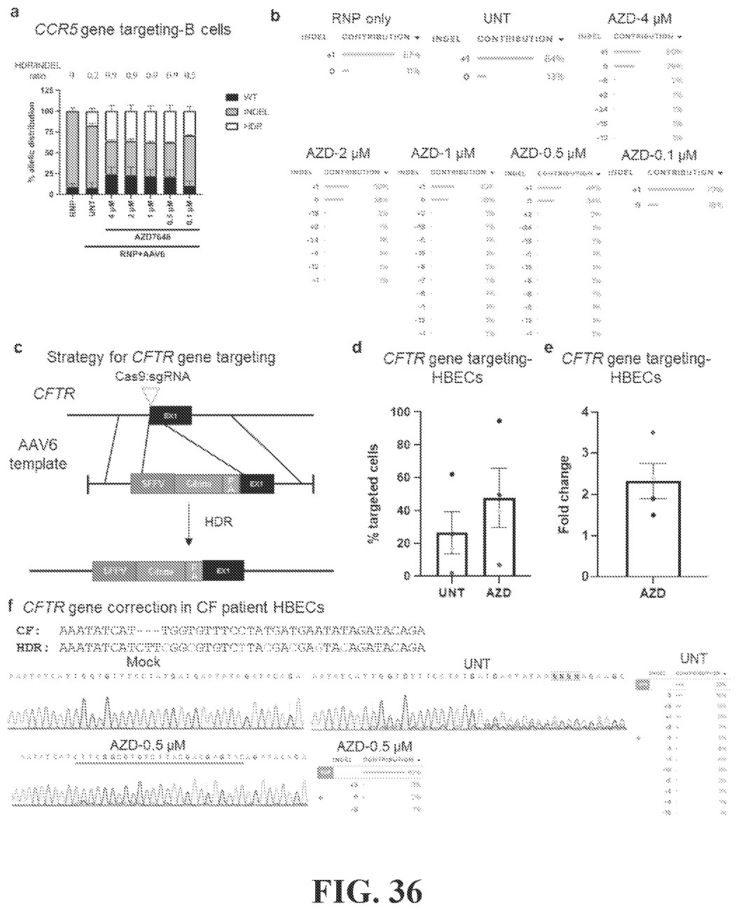

We tested gene targeting at the CCR5 locus in B cells with different concentrations of AZD7648. We observed a 2-fold increase in gene targeting efficiency with AZD7648 between the concentrations of 4 to 0.5 μM and a 1.7-fold increase at 0.1 μM. This improvement in gene targeting frequency and HDR to INDEL ratio in B cells was moderate at this locus when compared to the other cell types tested (FIG. 36a). In the non-gene targeted alleles, AZD7648 treatment at 4, 2, 1, 0.5 μM showed an increase in the frequency of WT alleles and a small fraction of alleles with deletion INDELs which was absent in the untreated cells. At 0.1 μM, AZD7648 treatment did not affect the pattern of INDELs in the non-gene targeted alleles similar to results in HSPCs and T cells (FIG. 36b).

Gene correction of CFTR mutation with AZD7648 in CF patient derived HBECs. We tested the effect of AZD7648 for gene targeting in primary human bronchial epithelial cells (HBECs). First, we performed an AZD7648 concentration gradient analysis in WT HBECs for editing the CF mutation, AF508, in exon 11 of the CFTR gene FIG. 19c, upper panel). Similar to other cell types, we found 0.5 μM as the minimum optimal concentration in HBECs for improving gene targeting as measured by allelic gene targeting efficiency (FIG. 14e). We then tested the effect of AZD7648 for gene targeting at exon 1 of the CFTR gene for integration of a 1 kb plus sequence in HBECs (FIG. 36c). Following gene targeting, we found that AZD7648 treatment improved the frequency of GFP+ cells by around 2-fold (FIG. 36d-e). Next, we tested the AZD7648 treatment for gene targeting to correct the AF508 mutation in CF-patient derived HBECs⁷³. The gene correction efficiency in the untreated cells was around 30%, which improved by 2.5-fold with AZD7648 treatment. Correspondingly, we observed an 18.5-fold increase in the HDR to INDEL ratio with the AZD7648 treatment indicating the dramatic drop in INDEL frequency (FIG. 14f, FIG. 36f). Importantly, the viable cell count was not affected in the gene targeted cells with AZD7648 treatment when compared to the untreated cells (FIG. 14g). Thus, these results show the applicability of AZD7648 treatment for correction of the most common CF mutation in patient derived HBECs.

Overall, these results confirm the applicability of AZD7648 treatment for improving gene targeting across different genomic loci in a wide variety of human primary cells.

### DISCUSSION

Despite the development of methods for improving targeted integration in human primary cells, the ratio of HDR-based targeted integration to INDELs often remains at 1 or lower, which is a limitation for the application of targeted integration for therapeutic and research purposes³². Small molecule-based inhibition of DNA-PKcs has been reported as a potential method for improving the frequency of HDR-based targeted integration through transient inhibition of the NHEJ pathway^{40, 41, 51, 55}. However, there is lack of a comprehensive evaluation of various commercially available small molecule DNA-PKcs inhibitors to identify the most potent and selective compound for improving gene targeting. This is particularly important as highly potent and selective small molecule DNA-PKcs inhibitors have been recently developed due to the relevance of DNA-PKcs as a target for anti-cancer therapy^{88, 89}. Following a screening of these recently developed compounds, we have identified AZD7648 as the most potent DNA-PKcs inhibitor for improving gene targeting efficiency. Transient inhibition of DNA-PKcs for 24 hours during gene editing with AZD7648 was sufficient to enhance the gene targeting efficiency with a concomitant increase in the HDR to INDEL ratio across different genomic loci in various therapeutically relevant human primary cells.

DNA-PKcs is a key player in the NHEJ-based repair of spontaneous and gene editing-induced DSB⁹⁰ and thus it is essential to confirm the effect of AZD7648 treatment on off-target editing as it could affect the genomic stability in the gene targeted cells. To confirm the safety of gene targeting with AZD7648 treatment, we assessed the off-target activity in gene targeted HSPCs and found a small increase of 2 to 3-fold in the frequency of off-target deletion INDELs potentially due to the activation of MMEJ pathway. Despite this increase in off-target activity, the high specificity of HiFi Cas9 based gene editing is largely maintained as it has been found to be 20 to 35-fold more specific than gene editing with the WT Cas9^{91, 92}. If there were a biologically important rationale, specific off-target INDELs can be blocked using a gRNA/dead Cas9 RNP to block that site^{79, 80}. Moreover, a previous study has shown that the genomic stability was largely maintained in human iPSCs with a catalytically inactive mutation in DNA-PKcs even after treatment with a DSB-inducing drug bleomycin or extensive passaging. Interestingly, this study showed that the genomic translocations induced by bleomycin drug treatment and number of mutations accumulated with passaging was fewer in the DNA-PKcs mutant human iPSCs than the WT cells⁵⁵. Finally, we note that these DNA-PKcs inhibitors have been given in vivo to humans as part of clinical trials without noted adverse events (NCT03907969, NCT02644278 and NCT02316197). Thus, transient (24 hours) small molecule-based inhibition of DNA-PKcs during gene editing may not affect the genomic integrity in the gene targeted cells.

PSCs hold great potential for applications such as disease modeling, drug screening and cell-based therapeutics^{93, 94}. Gene targeting of PSCs is broadly relevant for realizing the full potential of these applications⁹⁵. Although the feasibility of single cell cloning allows for the purification of gene targeted PSCs, low gene targeting efficiency often results in a long and tedious process to isolate the clones with desired edits. With the AZD7648 treatment we consistently achieved 50 to 90% allelic gene targeting efficiency in PSCs with low to no INDELs across different genomic loci. This high gene targeting efficiency will make it easier to screen and isolate bi-allelic gene targeted single cell PSC clones.

Therapeutic gene editing of HSPCs for autologous cell therapy holds the potential for treatment of currently incurable diseases of the blood and immune system^{71, 74, 75, 96}. Gene targeted HSPC-based autologous cell therapy has already entered clinical trials for sickle cell disease (NCT04819841)⁷². However, xenograft studies assessing the engraftment potential have shown that the gene targeted HSPCs possess a lower long-term engraftment capacity which could potentially limit the therapeutic benefit^{66, 71, 81, 97}. One possible explanation for this could be the lower levels of gene targeting in the quiescent stem cells with long-term engraftment capacity. Since, AZD7648 treatment significantly enhances targeted integration frequency in both bulk HSPCs and long-term hematopoietic stem cells, it may also increase the frequency of gene targeted cell engraftment, especially since AZD7648 allows lower amounts of both AAV6 and Cas9-RNP to be used while maintaining high levels of gene targeting. In addition, this enhanced gene targeting approach can also be useful for research applications such as disease modeling with HSPCs without the need for selection and enrichment of the targeted cells.

AZD7648 treatment also improved gene targeting in other primary cell types such as T cells, B cells and HBECs which shows the broad relevance of this approach across different cell types. Gene targeting in T cells is highly relevant for developing cancer immunotherapies⁹⁸ and here we have shown that AZD7648 treatment can improve T cell gene targeting at therapeutically relevant CCR5⁹⁹ and TRAC loci^{87, 100}. HBECs are being considered as a potential cell source for developing autologous cell therapies to treat CF^{73, 101, 102} Gene targeting with AZD7648 treatment improved the gene correction efficiency of the most common CF mutation at the CFTR locus (AF508) by several fold in patient derived HBECs⁷³. Thus, this gene targeting approach can improve the therapeutic efficacy of the potential ex-vivo gene targeted autologous cell-based therapies for CF.

One potential challenge associated with Cas9/gRNA-based gene editing is to find an active gRNA that creates high rates of INDELs near the genomic target site for achieving high efficiency gene targeting¹⁰³. Here, we have shown that application of AZD7648 treatment for gene editing with seemingly inactive and low activity gRNAs can result in high gene targeting efficiency of ˜50% in both human PSCs and HSPCs. Remarkably, using a low active gRNAs, high frequencies of gene targeting were achieved with low to no INDELs. Thus, this gene targeting approach could expand the number of gRNAs that can be used for gene targeting due to the possibility of rescuing seemingly inactive gRNAs. This will be particularly important for contexts where there is limited availability of gRNA target sites near the genomic region of interest.

Furthermore, we found that AZD7648 treatment allows for titrating down the amount of Cas9 RNP and AAV6 donor without compromising the gene targeting efficiency. Reducing the AAV6 dose for gene targeting can help reduce the toxicity associated with AAV transduction in primary cells⁶⁸,78. Although the off-target activity associated with Cas9-gRNA gene editing is significantly reduced through the use of the high-fidelity version of Cas9 nuclease⁹¹, the use of lower amounts of high-fidelity Cas9 and gRNA for gene editing could further reduce the off-target activity. Thus, titrating down the amounts of Cas9 RNP and AAV6 together to define the optimal amounts for gene targeting with AZD7648 could help achieve high gene targeting with reduced toxicity and off-target effects.

In conclusion, we have developed a gene targeting approach for achieving high gene targeting efficiency for precise knock-in of short to large sequences with low to no INDELs across different genomic loci in various human primary cells. This approach will broaden the application of targeted integration in human cells as a tool for therapeutic and research applications.

### Methods

DNA-PKcs inhibitors. Small molecule DNA-PKcs inhibitors used in this study are all commercially available. AZD7648 (Cat: S8843) and M3184 (Cat: S8586) were from Selleck Chemicals. VX984 (Cat: HY-19939S), KU57788 (Cat: HY-11006), LTURM34 (Cat: HY-101667) and BAY8400 (Cat: HY-132293) were from MedChemExpress. All DNA-PKcs inhibitors were resuspended in DMSO to make either 2 mM or 4 mM stocks and were diluted in cell culture medium to make up the indicated final concentrations for gene targeting.

Cas9 and sgRNA used for genome editing. High-fidelity Cas9⁹¹ purchased from Aldevron (SpyFi Cas9, Cat: 9214) was used for all genome editing experiments. gRNAs used for genome editing were purchased from either Synthego or TriLink Biotechnologies. gRNAs were chemically modified to include 2′-O-methyl-3′-phosphorothioate at the first and last 3 nucleotides, as described previously¹° 4. Following are the genomic target sites for the different gRNAs used in this study,

CCR5 (sg11):
5′-GCAGCATAGTGAGCCCAGAA-3′,
CCR5 (sg1):
5′-TCCTTCTTACTGTCCCCTTC-3′,
CCR5 (sg4):
5′-GGCAGCATAGTGAGCCCAGA-3′,
HBB:
5′-CTTGCCCCACAGGGCAGTAA-3′,
HBA1:
5′-GGCAAGAAGCATGGCCACCG-3′,
HBG1/2:
5′-CTTGTCAAGGCTATTGGTCA-3′
STING1-sg3:
5′-ACACTGCAGAGATCTCAGCT-3′,
STING1-sg5:
5′-CACACTGCAGAGATCTCAGC-3′,
IL2RG-sg1:
5′-TGGTAATGATGGCTTCAACA-3′,
IL2RG-sg5:
5′-ATTCCTGCAGCTGCCCCTGC-3′,
IL2RG-sg6:
5′-CGACAATTCTGACGCCCAAT-3′,
IL2RG-sg7:
5′-AGCTGCCCCTGCTGGGAGTG-3′,
TRAC:
5′-GAGAATCAAAATCGGTGAAT-3′
CFTR-Ex11 (AF508):
5′-TCTGTATCTATATTCATCAT-3′
CFTR-Ex1:
5′-TTCCAGAGGCGACCTCTGCA-3′

AAV6 vector construction, production and purification. For construction of the AAV transfer plasmid, pAAV-MCS plasmid (Agilent) backbone was used. pAAV-MCS plasmid was digested with NotI-HF enzyme (NEB) and sequences of the homology arms and insert were cloned into the backbone using NEBuilder® HiFi DNA Assembly Master Mix (NEB, cat: E2621L). After the cloning, transfer plasmids were sequence verified and purified using PureLink™ Expi Endotoxin-Free Maxi Plasmid Purification Kit (Thermo Fisher Scientific, Cat: A31217). AAV6 vectors were either produced in-house or acquired through Vigene or Signagen. For in-house production, 293T cells were seeded in five to ten 150-mm dishes at 10 million cells per dish. After 24 h, each dish with 293T cells was transfected with 22 μg of packaging/helper plasmid, pDGM6 (Gift from David Russell, Addgene plasmid #110660) and 6 μg of the transfer plasmid in 1 ml of OptiMEM I (Gibco, cat: 31985088) using PEI (polysciences, cat: 23966-1). 72 h post-transfection, AAV6 was purified using the AAVpro purification kit (Takara, cat: 6666) as per manufacturer's instructions. AAV6 titer was determined by droplet digital PCR (ddPCR) as per manufacturer's instructions using previously described primer/probe set¹⁰⁵.

PSC culture and genome editing. Gene editing experiments were performed with three PSC lines: H9 embryonic stem cells (ESC) (WiCell) and the previously described 1205-4 and 1208-2 iPS cell lines¹⁰⁶. PSCs were maintained in feeder free conditions on Matrigel (corning, cat: 354277) coated plates in mTeSR1 medium (STEMCELL technologies, cat: 85850). For gene editing, 24 h prior to nucleofection PSCs were pretreated with 10 μM of Y27632 (Cayman Chemical, cat: 10005583). For each nucleofection, RNP complex was prepared with 5 μg of Cas9 protein and 2 μg of gRNA and incubated for 15 mins at room temperature. PSCs were dissociated into single cells using Accutase (Innovative Cell Technologies, cat: AT104). 500,000 cells were resuspended with the RNP complex diluted in 20 μl of P3 primary cell nucleofector solution (Lonza, cat: V4XP-3032). Resuspended cells were added to one well of a 16-well Nucleocuvette Strip (Lonza) and nucleofection was performed in the 4D nucleofector (Lonza) using the program, CA137. Nucleofected PSCs were plated in mTeSR1 medium supplemented with 10 μM of Y27632 at a density of 100,000 cells per well in 48-well plate, AAV6 vector and DNA-PKcs inhibitors were added to the medium at indicated concentrations. After 24 h incubation, the existing medium was removed and fresh mTeSR1 supplemented with 10 μM of Y27632 was added to the cells. From the following day, PSCs were cultured in mTeSR1 without Y27632. Gene targeting efficiency was analyzed at 4-6 days post-editing.

For single cell cloning, gene targeted PSCs were plated at a density of 250 cells per well of a 6-well plate in mTeSR1 medium supplemented with 1× CloneR™2 (STEMCELL technologies, cat: 100-0691) and incubated at 37° C. for 2 days. After 2 days, medium was switched to fresh mTeSR1 medium supplemented with 1× CloneR™2. 2 days later, the cells were switched to and maintained in mTeSR1 medium with daily media changes. At D7-D10, single cell colonies were picked by scraping and were then propagated individually. Gene targeting was assessed in single cell clones by ddPCR as mentioned above and by PCR amplifying the region spanning the knock-in to determine the frequency of mono-, bi-allelic and non-targeted clones.

Cell viability measurement. MTT assay was used for measuring the gene edited PSC viability. Gene edited PSCs were plated in 96-well plates and cell viability was assessed at 24 h, 48 h and 72 h post editing. For this, 0.5 mg/ml of MTT (Cat: HY-15924, MedChemExpress) diluted in the growth medium was added to the cells and the plates were incubated at 37° C. for 2 hours. After this incubation, MTT was removed, and cells were lysed using 100 μl of lysis buffer (0.1N HCl and 0.5% SDS in isopropanol). After lysis, absorbance was measured at 570 nm with 650 nm as the reference using a SpectraMax M3 plate reader (Molecular devices). The absorbance values were used to calculate the cell viability of gene edited cells as a percentage relative to the mock cells. For measuring the viability of HSPCs, T cells and HBECs, cell suspension was mixed with Trypan Blue (Gibco, cat: 15250061) and viable cell counts were determined using TC10 cell counter (Biorad) at 3-5 days post gene editing. Viable cell counts were represented as percentage relative to the mock or untreated cells as indicated.

Pluripotency marker analysis. Mock, RNP only and gene targeted PSCs were assessed for the expression of pluripotency markers, SSEA4, OCT3/4, SOX2 and NANOG. For SSEA4 expression analysis, dissociated PSCs were stained with Alexa Fluor 647 conjugated anti-SSEA4 antibody (Biolegend, cat: 330407) and flow cytometry was used to measure the percentage of SSEA4 positive cells. OCT3/4, SOX2 and NANOG expression was assessed following immunofluorescence staining of fixed PSCs. For this, PSCs were fixed with 4% paraformaldehyde (Electron Microscopy Sciences, cat: 15710) for 20 mins at room temperature. Cells were then permeabilized with 0.3% Triton X-100 (Thermo Fisher Scientific, cat: 85111) for 20 mins at room temperature and then incubated with a blocking solution of 3% bovine serum albumin (BSA) (Gold biotechnology, cat: A-420-10) in PBS for 1 hour at room temperature. After blocking, cells were incubated with primary antibody diluted at 1:200 in 3% BSA and incubated at 4° C. overnight. Primary antibodies against OCT3/4 (cat: sc-5279), SOX2 (cat: sc-365823) and NANOG (cat: sc-293121) were all from Santa Cruz Biotechnology. Following day, cells were washed once with PBS and then incubated for 45 mins at room temperature with Alexa Fluor-647 conjugated anti-mouse secondary antibody (Thermo Fisher Scientific, cat: A21235) and DAPI (Santa Cruz Biotechnology, cat: sc-3598) diluted at 1:500 and 1:5000 respectively in 3% BSA. After this incubation, cells were washed once with PBS and were stored in fresh PBS at 4° C. until imaging. The stained cells were imaged using BZ-X710 microscope (Keyence) and the images were analyzed using the ImageJ software (NIH). The area of Alexa Fluor-647 and DAPI staining were measured following manual thresholding and the ratio of these areas was used to determine the percentage of cells positive for the corresponding markers. The percentage values were capped at 100% to account for the staining artifacts.

Trilineage differentiation of PSCs and analysis. Gene targeted PSCs (H9 ESC and 1205-4 iPSC) were assessed for three germ layer differentiation potential using STEMdiff™ Trilineage Differentiation Kit (STEMCELL Technologies, cat: 05230) as per manufacturer's instructions. Following the differentiation, cells were dissociated with Accutase, stained with antibodies against respective extracellular markers and then the cells were fixed and permeabilized using the eBioscience™ Foxp3/Transcription Factor Staining Buffer Set (Thermo Fisher Scientific, cat: 00-5523-00) and stained with antibodies against intracellular markers following manufacturer's instructions. Following are the primary antibodies used for staining, ectoderm: PAX6 (BD Biosciences, cat: BDB562249) and NES (Biolegend, cat: 656805), mesoderm: CD56 (Biolegend, cat: 318305) and T (R&D systems, cat: IC2085A), endoderm: CXCR4 (Biolegend, cat: 306505) and SOX17 (BD Biosciences, cat: BDB562594). Flow cytometry analysis was used to measure the percentage of cells double positive for the corresponding markers of the three germ layers.

ddPCR analysis of targeted integration. Gene targeting efficiency for the knock-in of multi-kb sequences was measured through digital droplet PCR (ddPCR) analysis. For this, genomic DNA was extracted from cells using the QuickExtract™ DNA Extraction Solution (QE) (Lucigen, Cat: QE09050). Pellets of 100,000 to 200,000 cells were resuspended in 40 to 50 μl of QE solution. This cell suspension was incubated at 65° C. for 6 mins and then 100° C. for 10 mins. The quick extracted genomic DNA was digested with either BamHI-HF or HindIII-HF (NEB) enzymes at 37° C. for 1 to 2 hours. 1-2 μl of the digested genomic DNA was used as a template for ddPCR reaction with 10 μl ddPCR supermix for probes (no dUTP) (Biorad, cat: 1863025), 0.5 μl each of fluorophore labeled target and reference primer/probe assays in a total volume of 20 μl. ddPCR droplets were generated in the droplet generator (Biorad) with 70 μl of droplet generation oil (Biorad, cat: 1863005) and 20 μl of sample as per manufacturer's instructions. 40 μl of the generated droplet sample was used for the PCR with the following cycle: 95° C. for 10 mins, 50 cycles of 94° C. for 30 secs, 60° C. for 30 secs and 72° C. for 2 mins and 98° C. for 10 mins. After PCR, the droplets were analyzed using the QX200 Droplet Digital PCR reader (Biorad) and Quantasoft software was used to determine the number of copies of the target and reference DNA in the droplets. The allelic gene targeting efficiency was calculated as the percentage of target DNA copies relative to that of the reference. The sequences of reference primer/probe assay specific to CCRL2 genomic sequence are as follows,

Forward primer (FP):
5′-GCTGTATGAATCCAGGTCC-3′,
Reverse primer (RP):
5′-CCTCCTGGCTGAGAAAAAG-3′
Probe:
5′-HEX/TGTTTCCTC/ZEN/CAGGATAAGGCAGCTGT/3IABKFQ-3′

The target primer/probe assays were designed such that one primer anneals to the insert and other primer anneals outside the homology arm (In-Out PCR). Following are the sequences of the different target primer/probe assays used,

CCR5-FP:
5′-GGGAGGATTGGGAAGACA-3′
CCR5-RP:
5′-AGGTGTTCAGGAGAAGGACA-3′
CCR5-probe:
5′-6-FAM/AGCAGGCAT/ZEN/GCTGGGGATGCGGTGG/
3IABKFQ-3′
HBB-FP:
5′-GGGAAGACAATAGCAGGCAT-3′
HBB-RP:
5′-CGATCCTGAGACTTCCACAC-3′
HBB-probe:
5′-6-FAM/TGGGGATGC/ZEN/GGTGGGCTCTATGGC/
3IABKFQ-3′
HBA1-YFP-FP:
5′-AGTCCAAGCTGAGCAAAGA-3′
HBA1-YFP-RP:
5′-TAGTGGGAACGATGGGGGAT-3′
HBA1-YFP-probe:
5′-6-FAM/CGAGAAGCG/ZEN/CGATCACATGGTCCTGC/
3IABKFQ-3′
HBA1-HBB-FP:
5′-GCTGCCTATCAGAAAGTGGT-3′
HBA1-HBB-RP:
5′-ATCACAAACGCAGGCAGAG-3′
HBA1-HBB-probe:
5′-6-FAM/CTGGTGTGG/ZEN/CTAATGCCCTGGCCC/
3IABKFQ-3′
IL2RG-target-FP:
5′-GGGTGACCAAGTCAAGGAAG-3′
IL2RG-target-RP:
5′-GATGGTGGTATTCAAGCCGA-3′
IL2RG-target-probe:
5′-6-FAM/CAAGCGCCA/ZEN/TGTTGAAACCCAGCCTGCCC/
3IABKFQ-3′
IL2RG-reference-FP:
5′-GGGAAGGTAAAACTGGCAAC-3′
IL2RG-reference-RP:
5′-GGGCACATATACAGCTGTCT-3′
IL2RG-reference-probe:
5′-5HEX/CCTCGCCAG/ZEN/TCTCAACAGGGACCCAGC/
3IABKFQ-3′

ICE analysis for quantification of gene editing. ICE CRISPR analysis tool (Synthego) was used to determine the allelic distribution of WT, INDEL and HDR frequencies for gene targeting involving knock-in of short sequences and for calculating the frequency of WT and INDEL alleles for gene targeting involving knock-in of multi-kb sequences. Quick extracted genomic DNA from mock and gene edited samples were used as a template for PCR to amplify the gene edited region using the PrimeSTAR GXL DNA Polymerase (Takara, cat: R050A). Following PCR, the amplicons were detected on agarose gel and purified from the gel using GeneJet Gel Extraction Kit (Thermo Fisher Scientific, cat: K0692). The purified DNA was Sanger sequenced through MCLAB or GENEWIZ. The sequencing chromatograms were analyzed through the ICE tool with mock sample sequence as the reference following manufacturer's instructions. Following are the sequences of primers used for PCR and Sanger sequencing,

CCR5 (stop codon):
FP:
5′-CATGACATTCATCTGTGGTGGC-3′
RP:
5′-TCTCATTTCGACACCGAAGC-3′
Sequencing primer (seq):
5′-GCACAGGGTGGAACAAGATGG-3′
CCR5 (UBC-GFP-bGH-pA):
FP:
5′-CTCATAGTGCATGTTCTTTGTGGGC-3′
RP:
5′-CCAGCCCAGGCTGTGTATGAAA-3′
Seq:
5′-GCACAGGGTGGAACAAGATGG-3′
HBB (SCD, UBC-GFP-bGHpA):
FP:
5′-AGGAAGCAGAACTCTGCACTTCA-3′
RP:
5′-AGTCAGTGCCTATCAGAAACCCAAGAG-3′
Seq:
5′-GAGGGAGGGCTGAGGGTTTGA-3′
CFTR (ΔF508)
FP:
5′-CCTTCTACTCAGTTTTAGTC-3′
RP:
5′-TGGGTAGTGTGAAGGGTTCAT-3′
Seq:
5′-AGGCAAGTGAATCCTGAGCG-3′
HBA1 (transgene-2A-YFP, HBB)
FP:
5′-TGCTTTTTGCGTCCTGGTGTT-3′
RP:
5′-AACGCCTGATCTTGACAGCCC-3′
Seq:
5′-AGATGGCGCCTTCCTCGC-3′
STING1 (V155M):
FP:
5′-CTCTCGCAGGCACTGAACAT-3′
RP:
5′-TCACTTTACCTCTCAGAACTGCAC-3′
Seq:
5′-GGTGTGACCTGCCCTGAGCTG-3′
HBG1/2 (13-bp del):
HBG1-FP and seq:
5′-AACCACTGCTAACTGAAAGAGACT-3′
HBG2-FP and seq:
5′-GCACTGAAACTGTTGCTTTATAGGAT-3′
HBG1/2 common-RP:5′-GGCGTCTGGACTAGGAGCTTATTG-3′
IL2RG (codon optimized cDNA-bGHpA):
FP:
5′-GGGTGACCAAGTCAAGGAAG-3′
RP:
5′-AATGTCCCACAGTATCCCTGG-3′
Seq:
5′-AGCCCGTGTCACACAGCACAT-3′

Cells were lysed with RIPA buffer (Thermo Fisher Scientific, cat: PI89900) supplemented with Halt Protease and Phosphatase Inhibitor Cocktail (Thermo Fisher Scientific, cat: PI78440) upon incubation for 15 mins at 4° C. Protein concentration was determined using BCA assay kit (Thermo Fisher Scientific, cat: PI23227) following manufacturer's instructions. Cell lysates were denatured by adding 1× Laemmli sample buffer (Bio-Rad, cat: 1610747) and heating at 100° C. for 5 mins. 20 μg of total protein was loaded on to 4-15% Mini-PROTEAN® TGX™ Precast Protein Gels (Bio-Rad, cat: 4561084) and electrophoresis was performed. Following electrophoresis, the protein was transferred on to PVDF membrane (Bio-Rad, cat: 1620177). The membrane was blocked in 5% dry milk in TBST for 1 hour at room temperature and then incubated with primary antibody diluted in 5% BSA in TBST at 4° C. Following day, the membrane was washed 3 times with TBST for 5 mins per wash and then incubated with HRP conjugated secondary antibody diluted in 5% dry milk for 45 mins at room temperature. After secondary antibody incubation, the membrane was washed 3 times with TBST for 5 mins per wash. For detection, membrane was incubated in SuperSignal™ West Femto Maximum Sensitivity Substrate (Thermo Fisher Scientific, cat: 34095) for 5 mins and the blot was imaged using Image Lab software. Following are the primary antibodies used: Anti-3-Actin antibody (BioLegend, cat: 643802), Anti-DNA-PKcs antibody (Cell Signaling Technology, cat: 38168S), Anti-DNA-PKcs (phospho-Ser2056) antibody (Abcam, cat: ab18192) and Anti-AKT (phosphor-Ser473) antibody (Cell Signaling Technology, cat: 4060S). Secondary antibodies used were, Anti-mouse IgG-HRP antibody (Cytiva, cat: NA931) and Anti-Rabbit IgG-HRP antibody (Cytiva, cat: NA934).

CD34+ HSPC culture and genome editing. Human CD34+ HSPCs were isolated from cord blood (provided by Binns program for Cord Blood Research). HSPCs were cultured in either StemSpan SFEM II (STEMCELL Technologies, cat: 09655) or SCGM (Cellgenix, cat: 20802) medium supplemented with 100 ng/ml of stem cell factor (PeproTech, cat: 300-07), 100 ng/ml of thrombopoietin (PeproTech, cat: 300-18), 100 ng/ml of FLT3-ligand (PeproTech, cat: 300-19), 100 ng/ml of IL-6 (PeproTech, cat: 200-06), 20 U/ml of penicillin, 20 mg/ml of streptomycin (Cytiva, cat: SV30010) and 35 nM of UM171 (APExBIO, cat: A89505). HSPCs were cultured at a cell density of 0.25-0.5 million cells/ml of the growth medium at 37° C., 5% 02 and 5% CO2. For gene editing of HSPCs, RNP complex was prepared with 6 μg of Cas9 and 3.2 μg of gRNA and incubated for 15 mins at room temperature and then diluted in 20 μl of P3 primary cell nucleofector solution (Lonza, cat: V4XP-3032). 0.5-1 million cells were resuspended in 20 μl of the RNP nucleofection solution and the nucleofection was performed using the program DZ-100. Nucleofected HSPCs were plated at the cell density mentioned above with the indicated amounts of AAV6 and AZD7648. After a 24 h incubation, cells were switched to fresh growth medium without AAV6 and AZD7648. Gene targeting efficiency was assessed at 3 days post-gene editing. For ssODN donor-based gene targeting experiments, 200 bp long ssODN Alt-R™ HDR Donor Oligos were used (IDT). ssODN was delivered into the cells through nucleofection. ssODN was added to the nucleofection solution with RNP at a concentration of 2.5 or 5 μM and HSPCs were nucleofected and cultured as mentioned above.

Colony forming units assay and genotyping of clones. For colony forming units (CFU) assay, 250 to 500 HSPCs were plated in SmartDish 6-well plates (STEMCELL Technologies, cat: 27370) containing MethoCult H4434 Classic (STEMCELL Technologies, cat: 04444). After 14 days, colonies were counted and scored using STEMvision automated counter (STEMCELL Technologies) to determine the number of BFU-E, CFU-M, CFU-GM, and CFU-GEMM colonies. To determine the ratio of monoallelic vs biallelic integration, individual colonies of each type were picked and genomic DNA was extracted with QE. ddPCR was used to determine the clones with mono- vs bi-allelic integration as mentioned. WT and INDEL frequencies in the clones were determined using ICE analysis.

FACS sorting of LT-HSC and MPP. Cord-blood derived human CD34+ HSPCs were gene targeted at the CCR5 locus for the knock-in of UBC-GFP-bGHpA sequence with and without AZD7648 treatment. Mock and AAV6 only cells were used as negative controls. 2 days post gene targeting, HSPCs were stained with a cocktail of antibodies: APC anti-CD34 (BioLegend, cat: 343510), BV785 anti-CD90 (BioLegend, cat: 328142), BV650 anti-CD38 (BioLegend, cat: 356619), BV605 anti-CD45RA (BioLegend, cat: 304134) and BV510 anti-Lineage cocktail (BioLegend, cat: 348807) after blocking with Human TruStain FcX (BioLegend, cat: 422301). Cell viability was assessed using LIVE/DEAD™ Fixable Near-IR Dead Cell Stain Kit (Thermo Fisher Scientific, cat: L10119). LT-HSC and MPP cells were isolated by FACS sorting for CD34+, CD90+, Lin−, CD45RA−, CD38−(LT-HSC) and CD34+, CD90−, Lin−, CD45RA−, CD38−(MPP) in FACS Aria II sorter part of the FACS core facility in the Stanford Institute for Stem Cell Biology and Regenerative Medicine. Allelic gene targeting efficiency in LT-HSC and MPP was measured by ddPCR analysis as described above. FACS data analysis was performed using FlowJo software.

NGS analysis for off-target effects. For this, we used commercially available Amplicon-EZ next generation sequencing service from AZENTA, Inc. Previously characterized top off-target site for CCR5 (OT39), HBB (OT1) and HBA1 (OT1) gRNAs were analyzed. PCR was used to amplify a 350-450 bp region encompassing the off-target sites from genomic DNA of the gene targeted HSPCs. Following are the primers used for PCR,

CCR5 (OT39, chromosome 14):
FP: 5′-CAGCCCAGCTTCTGAGTTTTATATG-3,
RP: 5′-TGTGTTGATGTCATCCTTGTCC-3′
HBB (OT1, chromosome 9):
FP: 5′-CACTGCATCAGAATCATTTGGAGAATC-3,
RP: 5′-GGAACCATGGGAAGCATGTGATGT-3′
HBA1 (OT1, chromosome 1):
FP: 5′-CTCTGACTCACCAACTGGGC-3,
RP: 5′-GCGTTTTCTCTTCTAGGGATCTGC-3′

Following Illumina adapter sequences were added to the forward and reverse primers for next generation sequencing,

Forward:
5′-ACACTCTTTCCCTACACGACGCTCTTCCGATCT-3′
Reverse:
5′-GACTGGAGTTCAGACGTGTGCTCTTCCGATCT-3′

Following PCR, the amplicons were purified using Genejet PCR purification kit (Thermo Fisher Scientific, cat: K0701). The samples were normalized to a concentration of 20 ng/μl and 25 μl volume was submitted for Amplicon-EZ NGS service (AZENTA, Inc). DNA library preparations, sequencing reactions, and bioinformatics analysis were conducted at AZENTA, Inc. DNA Library Preparation was done using NEBNext Ultra DNA Library Prep kit following the manufacturer's recommendations (NEB). DNA amplicons were indexed and enriched by limited cycle PCR. DNA libraries were validated on the Agilent TapeStation (Agilent Technologies) and were quantified using Qubit 2.0 Fluorometer (Invitrogen) and multiplexed in equal molar mass. The pooled DNA libraries were loaded on the Illumina instrument according to manufacturer's instructions. The samples were sequenced using a 2×250 paired-end (PE) configuration. Image analysis and base calling were conducted by the Illumina Control Software on the Illumina instrument. The raw Illumina reads were checked for adapters and quality via FastQC. The raw Illumina sequence reads were trimmed of their adapters and nucleotides with poor quality using Trimmomatic v.0.36. Paired sequence reads were then merged to form a single sequence if the forward and reverse reads were able to overlap. The merged reads were aligned to the reference sequence and variant detection was performed using AZENTA proprietary Amplicon-EZ program. INDEL frequency at the target site was quantified and plotted as the frequency of reads with insertions and deletions.

T cell culture and genome editing. Leukocyte reduction system (LRS) chambers from healthy donors (Stanford Blood Center) were used for isolation of T cells. Peripheral blood mononuclear cells (PBMCs) isolated on a Ficoll density gradient were used for obtaining T cells using the CD4+ T Cell Isolation Kit (Miltenyi, cat: 130-096-533). For CD19-CAR targeting experiments, isolated αβ+ T cells were used. T cells were cultured in X-VIVO 15 media (Lonza, cat: 04-418Q) supplemented with 5% human AB serum (Sigma, cat: H3667) and 100 IU/ml recombinant human IL-2 (PeproTech, cat: 200-02) at 37° C., ambient 02 and 5% CO2. Medium changes were performed every 2-4 days and cultured cells were maintained at a target density of 0.5-1 million cells/ml unless otherwise indicated. T cells were activated with Dynabeads Human T Cell Activator (Gibco, cat: 11161D) for 72-96 hours and beads were removed before nucleofection. For gene targeting, electroporation was performed as previously described⁸⁷. gRNA was complexed with Cas9 at a molar ratio of 2.5:1 (gRNA:protein) and nucleofected in P3 primary cell nucleofector solution (Lonza) into activated T cells using a 4D-Nucleofector (Lonza) in 16-well cuvette strips. 1 million activated T cells were used per nucleofection using the program EO-115. The cells were resuspended directly after nucleofection in 80 μl of complete T cell medium and then diluted to the target density. For gene targeting, cells were incubated within 15 minutes after electroporation with AAV6 for transduction at the indicated MOI with or without the AZD7648. After 3-4 h, the suspension was diluted with complete medium to reach the target cell concentration of 1 million cells/ml. After a 24 h incubation, cells were switched to fresh medium. Gene targeting was analyzed at 3-4 days post gene editing.

CAR T cell cytotoxicity assay. CD19-directed CAR T cells generated in the presence or absence of AZD7648 were challenged with target CD19-expressing GFP+ Nalm6 leukemia cell line. The effector and target cells were cocultured at a ratio of 1:1 in RPMI medium supplemented with 10% bovine growth serum. The cytotoxicity effect of the CAR T cells or the depletion of target leukemia cells was monitored daily over two days by measuring the levels of GFP+ cells by flow cytometry using Beckman Coulter Accuri or CytoFLEX flow cytometer.

B cell culture and genome editing. Primary human B cells were isolated from LRS chambers obtained from the Stanford Blood Center via negative selection using a human B Cell Isolation Kit II (Miltenyi Biotec, cat: 130-091-151) according to manufacturer's instructions. Cells were cultured in Iscove's modified Dulbecco's medium (IMDM) (Thermo Fisher Scientific, cat: 12440053) supplemented with 10% bovine growth serum (Hyclone, cat: SH30541.03HI), 1% penicillin-streptomycin (Cytiva, cat: SV30010), 55 μM of 2-mercaptoethanol (Sigma-Aldrich, cat: M3148), 50 ng/ml of IL-2 (Peprotech, cat: 200-02), 50 ng/ml of IL-10 (Peprotech, cat: 200-10), 10 ng/ml of IL-15 (Peprotech, cat: 200-15), 100 ng/ml of recombinant human MEGACD40L (Enzo Life Sciences, cat: ALX-522-110-CO10), and 1 μg/ml of CpG oligonucleotide 2006 (Invivogen, cat: tlrl-2006-1) at a density of 1 million cells/ml, as described previously¹⁰⁷. B cells were cultured at 37° C., 5% CO2, and ambient oxygen levels.

For genome editing, gRNA targeting CCR5 was complexed with Cas9 protein were complexed at a 2.5:1 (Cas9: gRNA) molar ratio at room temperature for 20 min. B cells were nucleofected 4-5 days after thawing using the Lonza Nucleofector 4D (program EO-117) using 1 million cells per condition. Immediately following nucleofection, cells were incubated with AAV6 donor vector (UBC-GFP) at a MOI of 25,000 and varying concentrations of AZD7648 in 100 ul of basal IMDM in a 96 well plate for 3-4 hours¹⁰⁸. Cells were then replated at 1 million cells/ml in complete B cell activation media. Approximately 24 hours after nucleofection, the cells were replated in fresh media to remove AZD7648. Gene targeting was assessed at 3-days post-editing.

Cell culture and genome editing of HBECs. Human bronchial epithelial cells (HBECs) were obtained from Lonza Inc., the Primary Airway Cell Biobank at McGill University, the Cystic Fibrosis Foundation Cell Bank or the University of North Carolina. HBECs were cultured in Pneumacult Ex-Plus medium (STEMCELL Technologies, cat: 05040) at 3,000-10,000 cells/cm2 in tissue culture flasks. Cells were cultured at 37° C. in 5% 02 and 5% CO2. The media was supplemented with 10 μM ROCK inhibitor (Y-27632, Santa Cruz Biotechnology, cat: sc-281642A).

For genome editing, HBECs were resuspended at a density of 5 million cells/mL in OptiMEM I. Nucleofection was performed using Lonza 4D 16-well Nucleocuvette™ Strips (Lonza, V4XP-3032). 6 μg of high fidelity Cas9 and 3.2 μg of sgRNA (molar ratio=1:2.5) were complexed at room temperature for 10 minutes and then mixed with 20 μl of cell suspension in OptiMEM I. Cells were electroporated using the program CA-137. AAV6 was added at an MOI of 100,000 and AZD7648 was added at indicated concentrations.

Statistical analysis. GraphPad Prism 9 software was used for all statistical analysis.

### REFERENCES

25. Carroll, D. Genome engineering with targetable nucleases. Annu Rev Biochem 83, 409-439 (2014).
26. Gaj, T., Gersbach, C. A. & Barbas, C. F., 3rd ZFN, TALEN, and CRISPR/Cas-based methods for genome engineering. Trends Biotechnol 31, 397-405 (2013).
27. Horvath, P. & Barrangou, R. CRISPR/Cas, the immune system of bacteria and archaea. Science 327, 167-170 (2010).
28. Doudna, J. A. & Charpentier, E. Genome editing. The new frontier of genome engineering with CRISPR-Cas. Science 346, 1258096 (2014).
29. Hsu, P. D., Lander, E. S. & Zhang, F. Development and applications of CRISPR-Cas9 for genome engineering. Cell 157, 1262-1278 (2014).
30. Cong, L. et al. Multiplex genome engineering using CRISPR/Cas systems. Science 339, 819-823 (2013).
31. Mali, P. et al. RNA-guided human genome engineering via Cas. Science 339, 823-826 (2013).
32. Nambiar, T. S., Baudrier, L., Billon, P. & Ciccia, A. CRISPR-based genome editing through the lens of DNA repair. Mol Cell 82, 348-388 (2022).
33. Betermier, M., Bertrand, P. & Lopez, B. S. Is non-homologous end-joining really an inherently error-prone process?PLoS Genet 10, e1004086 (2014).
34. Kanaar, R., Hoeijmakers, J. H. & van Gent, D. C. Molecular mechanisms of DNA double strand break repair. Trends Cell Biol 8, 483-489 (1998).
35. Chang, H. H. Y., Pannunzio, N. R., Adachi, N. & Lieber, M. R. Non-homologous DNA end joining and alternative pathways to double-strand break repair. Nat Rev Mol Cell Biol 18, 495-506 (2017).
36. Sfeir, A. & Symington, L. S. Microhomology-Mediated End Joining: A Back-up Survival Mechanism or Dedicated Pathway?Trends Biochem Sci 40, 701-714 (2015).
37. Seol, J. H., Shim, E. Y. & Lee, S. E. Microhomology-mediated end joining: Good, bad and ugly. Mutat Res 809, 81-87 (2018).
38. Doudna, J. A. The promise and challenge of therapeutic genome editing. Nature 578, 229-236 (2020).
39. Porteus, M. H. A New Class of Medicines through DNA Editing. N Engl J Med 380, 947-959 (2019).
40. Fu, Y. W. et al. Dynamics and competition of CRISPR-Cas9 ribonucleoproteins and AAV donor-mediated NHEJ, MMEJ and HDR editing. Nucleic Acids Res 49, 969-985 (2021).
41. Tatiossian, K. J. et al. Rational Selection of CRISPR-Cas9 Guide RNAs for Homology-Directed Genome Editing. Mol Ther 29, 1057-1069 (2021).
42. Hustedt, N. & Durocher, D. The control of DNA repair by the cell cycle. Nat Cell Biol 19, 1-9 (2016).
43. Xue, C. & Greene, E. C. DNA Repair Pathway Choices in CRISPR-Cas9-Mediated Genome Editing. Trends Genet 37, 639-656 (2021).
44. Ray, U. & Raghavan, S. C. Modulation of DNA double-strand break repair as a strategy to improve precise genome editing. Oncogene 39, 6393-6405 (2020).
45. Sun, W. et al. Strategies for Enhancing the Homology-Directed Repair Efficiency of CRISPR-Cas Systems. CRISPR J 5, 7-18 (2022).
46. Bischoff, N., Wimberger, S., Maresca, M. & Brakebusch, C. Improving Precise CRISPR Genome Editing by Small Molecules: Is there a Magic Potion? Cells 9 (2020).
47. Fattah, F. et al. Ku regulates the non-homologous end joining pathway choice of DNA double-strand break repair in human somatic cells. PLoS Genet 6, e1000855 (2010).
48. Frit, P., Ropars, V., Modesti, M., Charbonnier, J. B. & Calsou, P. Plugged into the Ku-DNA hub: The NHEJ network. Prog Biophys Mol Biol 147, 62-76 (2019).
49. Zhao, B. et al. The essential elements for the noncovalent association of two DNA ends during NHEJ synapsis. Nat Commun 10, 3588 (2019).
50. Chu, V. T. et al. Increasing the efficiency of homology-directed repair for CRISPR-Cas9-induced precise gene editing in mammalian cells. Nat Biotechnol 33, 543-548 (2015).
51. Robert, F., Barbeau, M., Ethier, S., Dostie, J. & Pelletier, J. Pharmacological inhibition of DNA-PK stimulates Cas9-mediated genome editing. Genome Med 7, 93 (2015).
52. Maruyama, T. et al. Increasing the efficiency of precise genome editing with CRISPR-Cas9 by inhibition of nonhomologous end joining. Nat Biotechnol 33, 538-542 (2015).
53. Pinder, J., Salsman, J. & Dellaire, G. Nuclear domain ‘knock-in’ screen for the evaluation and identification of small molecule enhancers of CRISPR-based genome editing. Nucleic Acids Res 43, 9379-9392 (2015).
54. Canny, M. D. et al. Inhibition of 53BP1 favors homology-dependent DNA repair and increases CRISPR-Cas9 genome-editing efficiency. Nat Biotechnol 36, 95-102 (2018).
55. Riesenberg, S. et al. Simultaneous precise editing of multiple genes in human cells. Nucleic Acids Res 47, e116 (2019).
56. Fok, J. H. L. et al. AZD7648 is a potent and selective DNA-PK inhibitor that enhances radiation, chemotherapy and olaparib activity. Nat Commun 10, 5065 (2019).
57. Martin, R. M. et al. Highly Efficient and Marker-free Genome Editing of Human Pluripotent Stem Cells by CRISPR-Cas9 RNP and AAV6 Donor-Mediated Homologous Recombination. Cell Stem Cell 24, 821-828 e825 (2019).
58. Goldberg, F. W. et al. The Discovery of 7-Methyl-2-[(7-methyl[1,2,4]triazolo[1,5-a]pyridin-6-yl)amino]-9-(tetrahydro-2H-p yran-4-yl)-7,9-dihydro-8H-purin-8-one (AZD7648), a Potent and Selective DNA-Dependent Protein Kinase (DNA-PK) Inhibitor. J Med Chem 63, 3461-3471 (2020).
59. Zenke, F. T. et al. Pharmacologic Inhibitor of DNA-PK, M3814, Potentiates Radiotherapy and Regresses Human Tumors in Mouse Models. Mol Cancer Ther 19, 1091-1101 (2020).
60. Khan, A. J. et al. VX-984 is a selective inhibitor of non-homologous end joining, with possible preferential activity in transformed cells. Oncotarget 9, 25833-25841 (2018).
61. Hardcastle, I. R. et al. Discovery of potent chromen-4-one inhibitors of the DNA-dependent protein kinase (DNA-PK) using a small-molecule library approach. J Med Chem 48, 7829-7846 (2005).
62. Berger, M. et al. BAY-8400: A Novel Potent and Selective DNA-PK Inhibitor which Shows Synergistic Efficacy in Combination with Targeted Alpha Therapies. J Med Chem 64, 12723-12737 (2021).
63. Morrison, R., Al-Rawi, J. M., Jennings, I. G., Thompson, P. E. & Angove, M. J. Synthesis, structure elucidation, DNA-PK and PI3K and anti-cancer activity of 8- and 6-aryl-substituted-1-3-benzoxazines. Eur J Med Chem 110, 326-339 (2016).
64. Liu, R. et al. Homozygous defect in HIV-1 coreceptor accounts for resistance of some multiply-exposed individuals to HIV-1 infection. Cell 86, 367-377 (1996).
65. Papapetrou, E. P. & Schambach, A. Gene Insertion Into Genomic Safe Harbors for Human Gene Therapy. Mol Ther 24, 678-684 (2016).
66. Gomez-Ospina, N. et al. Human genome-edited hematopoietic stem cells phenotypically correct Mucopolysaccharidosis type I. Nat Commun 10, 4045 (2019).
67. Scharenberg, S. G. et al. Engineering monocyte/macrophage-specific glucocerebrosidase expression in human hematopoietic stem cells using genome editing. Nat Commun 11, 3327 (2020).
68. Brown, N., Song, L., Kollu, N. R. & Hirsch, M. L. Adeno-Associated Virus Vectors and Stem Cells: Friends or Foes?Hum Gene Ther 28, 450-463 (2017).
69. Tsuchiya, A., Kanno, T. & Nishizaki, T. PI3 kinase directly phosphorylates Akt1/2 at Ser473/474 in the insulin signal transduction pathway. J Endocrinol 220, 49-59 (2014).
70. Chen, J., Ghorai, M. K., Kenney, G. & Stubbe, J. Mechanistic studies on bleomycin-mediated DNA damage: multiple binding modes can result in double-stranded DNA cleavage. Nucleic Acids Res 36, 3781-3790 (2008).
71. Dever, D. P. et al. CRISPR/Cas9 beta-globin gene targeting in human haematopoietic stem cells. Nature 539, 384-389 (2016).
72. Lattanzi, A. et al. Development of beta-globin gene correction in human hematopoietic stem cells as a potential durable treatment for sickle cell disease. Sci Transl Med 13 (2021).
73. Vaidyanathan, S. et al. High-Efficiency, Selection-free Gene Repair in Airway Stem Cells from Cystic Fibrosis Patients Rescues CFTR Function in Differentiated Epithelia. Cell Stem Cell 26, 161-171 e164 (2020).
74. Cromer, M. K. et al. Gene replacement of alpha-globin with beta-globin restores hemoglobin balance in beta-thalassemia-derived hematopoietic stem and progenitor cells. Nat Med 27, 677-687 (2021).
75. Pavel-Dinu, M. et al. Gene correction for SCID-X1 in long-term hematopoietic stem cells. Nat Commun 10, 1634 (2019).
76. Romero, Z. et al. Editing the Sickle Cell Disease Mutation in Human Hematopoietic Stem Cells: Comparison of Endonucleases and Homologous Donor Templates. Mol Ther 27, 1389-1406 (2019).
77. Pavani, G. et al. Ex vivo editing of human hematopoietic stem cells for erythroid expression of therapeutic proteins. Nat Commun 11, 3778 (2020).
78. Dudek, A. M. & Porteus, M. H. Answered and Unanswered Questions in Early-Stage Viral Vector Transduction Biology and Innate Primary Cell Toxicity for Ex-Vivo Gene Editing. Front Immunol 12, 660302 (2021).
79. Rose, J. C. et al. Suppression of unwanted CRISPR-Cas9 editing by co-administration of catalytically inactivating truncated guide RNAs. Nat Commun 11, 2697 (2020).
80. Coelho, M. A. et al. CRISPR GUARD protects off-target sites from Cas9 nuclease activity using short guide RNAs. Nat Commun 11, 4132 (2020).
81. DeWitt, M. A. et al. Selection-free genome editing of the sickle mutation in human adult hematopoietic stem/progenitor cells. Sci Transl Med 8, 360ra134 (2016).
82. Cavazzana, M., Antoniani, C. & Miccio, A. Gene Therapy for beta-Hemoglobinopathies. Mol Ther 25, 1142-1154 (2017).
83. Gilman, J. G. et al. Distal CCAAT box deletion in the A gamma globin gene of two black adolescents with elevated fetal A gamma globin. Nucleic Acids Res 16, 10635-10642 (1988).
84. Chen, F. et al. High-frequency genome editing using ssDNA oligonucleotides with zinc-finger nucleases. Nat Methods 8, 753-755 (2011).
85. Traxler, E. A. et al. A genome-editing strategy to treat beta-hemoglobinopathies that recapitulates a mutation associated with a benign genetic condition. Nat Med 22, 987-990 (2016).
86. Liu, Y. et al. Activated STING in a vascular and pulmonary syndrome. N Engl J Med 371, 507-518 (2014).
87. Wiebking, V. et al. Genome editing of donor-derived T-cells to generate allogenic chimeric antigen receptor-modified T cells: Optimizing alphabeta T cell-depleted haploidentical hematopoietic stem cell transplantation. Haematologica 106, 847-858 (2021).
88. Mohiuddin, I. S. & Kang, M. H. DNA-PK as an Emerging Therapeutic Target in Cancer. Front Oncol 9, 635 (2019).
89. Hu, S. et al. Small molecule DNA-PK inhibitors as potential cancer therapy: a patent review (2010-present). Expert Opin Ther Pat 31, 435-452 (2021).
90. Yue, X., Bai, C., Xie, D., Ma, T. & Zhou, P. K. DNA-PKcs: A Multi-Faceted Player in DNA Damage Response. Front Genet 11, 607428 (2020).
91. Vakulskas, C. A. et al. A high-fidelity Cas9 mutant delivered as a ribonucleoprotein complex enables efficient gene editing in human hematopoietic stem and progenitor cells. Nat Med 24, 1216-1224 (2018).
92. Cromer, M. K. et al. Comparative analysis of CRISPR off-target discovery tools following ex vivo editing of CD34(+) hematopoietic stem and progenitor cells. Mol Ther (2023).
93. Shi, Y., Inoue, H., Wu, J. C. & Yamanaka, S. Induced pluripotent stem cell technology: a decade of progress. Nat Rev Drug Discov 16, 115-130 (2017).
94. Yamanaka, S. Pluripotent Stem Cell-Based Cell Therapy-Promise and Challenges. Cell Stem Cell 27, 523-531 (2020).
95. Hockemeyer, D. & Jaenisch, R. Induced Pluripotent Stem Cells Meet Genome Editing. Cell Stem Cell 18, 573-586 (2016).
96. Sweeney, C. L. et al. Correction of X-CGD patient HSPCs by targeted CYBB cDNA insertion using CRISPR/Cas9 with 53BP1 inhibition for enhanced homology-directed repair. Gene Ther 28, 373-390 (2021).
97. Genovese, P. et al. Targeted genome editing in human repopulating haematopoietic stem cells. Nature 510, 235-240 (2014).
98. June, C. H., O'Connor, R. S., Kawalekar, O. U., Ghassemi, S. & Milone, M. C. CAR T cell immunotherapy for human cancer. Science 359, 1361-1365 (2018).
99. Sather, B. D. et al. Efficient modification of CCR5 in primary human hematopoietic cells using a megaTAL nuclease and AAV donor template. Sci Transl Med 7, 307ra156 (2015).
100. Eyquem, J. et al. Targeting a CAR to the TRAC locus with CRISPR/Cas9 enhances tumour rejection. Nature 543, 113-117 (2017).
101. Berical, A., Lee, R. E., Randell, S. H. & Hawkins, F. Challenges Facing Airway Epithelial Cell-Based Therapy for Cystic Fibrosis. Front Pharmacol 10, 74 (2019).
102. Vaidyanathan, S. et al. Targeted replacement of full-length CFTR in human airway stem cells by CRISPR-Cas9 for pan-mutation correction in the endogenous locus. Mol Ther 30, 223-237 (2022).
103. Wilson, L. O. W., O'Brien, A. R. & Bauer, D. C. The Current State and Future of CRISPR-Cas9 gRNA Design Tools. Front Pharmacol 9, 749 (2018).
104. Hendel, A. et al. Chemically modified guide RNAs enhance CRISPR-Cas genome editing in human primary cells. Nat Biotechnol 33, 985-989 (2015).
105. Aurnhammer, C. et al. Universal real-time PCR for the detection and quantification of adeno-associated virus serotype 2-derived inverted terminal repeat sequences. Hum Gene Ther Methods 23, 18-28 (2012).
106. Khan, T. A. et al. Neuronal defects in a human cellular model of 22q.2 deletion syndrome. Nat Med 26, 1888-1898 (2020).
107. Hung, K. L. et al. Engineering Protein-Secreting Plasma Cells by Homology-Directed Repair in Primary Human B Cells. Mol Ther 26, 456-467 (2018).
108. Rogers, G. L. et al. Optimization of AAV6 transduction enhances site-specific genome editing of primary human lymphocytes. Mol Ther Methods Clin Dev 23, 198-209 (2021).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### 10. Exemplary Embodiments

Exemplary embodiments provided in accordance with the presently disclosed subject matter include, but are not limited to, the embodiments and the following embodiments:

Embodiment 1. A method of genetically modifying a primary human cell, the method comprising:
(i) introducing into the cell a site-directed nuclease (SDN) targeted to a cleavage site at a genetic locus of interest;
(ii) introducing a homologous donor template into the cell, wherein the homologous donor template comprises a nucleotide sequence that is homologous to the locus of interest; and
(iii) introducing a DNA-PK inhibitor into the cell,
wherein the site-directed nuclease cleaves the locus at the cleavage site, and the homologous donor template is integrated at the site of the cleaved locus by homology directed repair (HDR).

Embodiment 2. The method of embodiment 1, wherein the DNA-PK inhibitor is a compound represented by the following formula:

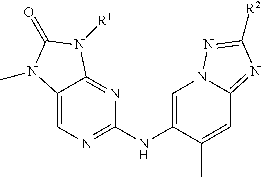

wherein:
R¹ is a cyclohexyl, tetrahydrofuranyl or oxanyl ring, each of which is optionally substituted by one or more groups selected from hydroxyl, methoxy, and methyl; and
R² is hydrogen or methyl,
or a pharmaceutically acceptable salt thereof.

Embodiment 3. The method of embodiment 2, wherein R¹ is oxanyl.

Embodiment 4. The method of embodiment 3, wherein R¹ is oxan-4-yl.

Embodiment 5. The method of any one of embodiments 2 to 4, wherein R² is hydrogen.

Embodiment 6. The method of any one of embodiments 2 to 5, wherein the DNA-PK inhibitor is AZD7648 represented by the following formula:

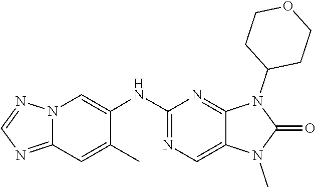 [SMILES: Cc1cc2ncnn2cc1Nc1ncc2c(n1)n(C1CCOCC1)c(=O)n2C]

or a pharmaceutically acceptable salt thereof.

Embodiment 7. The method of embodiment 1, wherein the DNA-PK inhibitor is VX984 represented by the following formula:

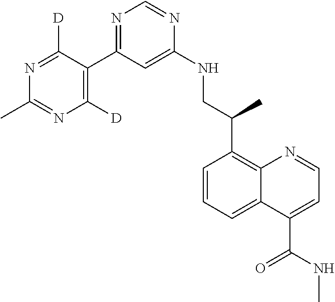

or a pharmaceutically acceptable salt thereof.

Embodiment 8. The method of embodiment 1, wherein the DNA-PK inhibitor is BAY8400 represented by the following formula:

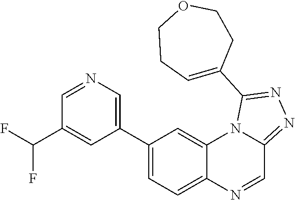 [SMILES: FC(F)c1cncc(-c2ccc3ncc4nnc(C5=CCCOCC5)n4c3c2)c1]

or a pharmaceutically acceptable salt thereof.

Embodiment 9. The method of any one of embodiments 1 to 8, wherein the DNA-PK inhibitor has very high specificity for the catalytic subunit of DNA-PK (DNA-PKcs).

Embodiment 10. The method of embodiment 9, wherein the DNA-PK inhibitor with very high specificity for DNA-PKcs has an IC50 in the range of about 40 nM to about 1 μM for DNA-PKcs and an IC50 of greater than 1 μM for other PIKK family kinases.

Embodiment 11. The method of embodiment 10, wherein the other PIKK family kinases are ATM, ATR, PI3Kα, PI3Kβ, PI3Kγ, PI3Kδ, and/or mTOR.

Embodiment 12. The method of any one of embodiments 1 to 11, wherein the SDN is an RNA-guided nuclease and the method further comprises introducing into the cell a single guide RNA (sgRNA) targeting the cleavage site, wherein the sgRNA directs the RNA-guided nuclease to the cleavage site.

Embodiment 13. The method of embodiment 12, wherein the sgRNA comprises 2′-O-methyl-3′-phosphorothioate (MS) modifications at one or more nucleotides.

Embodiment 14. The method of embodiment 13, wherein the MS modifications are present at the terminal nucleotides of the 5′ and 3′ ends.

Embodiment 15. The method of any one of embodiments 12 to 14, wherein the RNA-guided nuclease is Cas9.

Embodiment 16. The method of any one of embodiments 12 to 15, wherein the sgRNA and RNA-guided nuclease are introduced into the cell as a ribonucleoprotein (RNP).

Embodiment 17. The method of embodiment 16, wherein the RNP is introduced into the cell by electroporation.

Embodiment 18. The method of any one of embodiments 12 to 17, wherein the sgRNA is introduced into cells at a concentration of less than about 150 μg/ml, 75 μg/ml, 30 μg/ml, or 15 μg/ml.

Embodiment 19. The method of any one of embodiments 12 to 18, wherein the RNA-guided nuclease is introduced into cells at a concentration of less than about 300 μg/ml, 150 μg/ml, 60 μg/ml, or 30 μg/ml.

Embodiment 20. The method of any one of embodiments 1 to 19, wherein the homologous repair template is introduced into the cell using an adeno-associated virus serotype 6 (AAV6) vector.

Embodiment 21. The method of embodiment 20, wherein the AAV6 vector is transduced into the cell at a multiplicity of infection (MOI) of less than about 2500, 1000, or 500.

Embodiment 22. The method of embodiment 21, wherein the MOI is about 500.

Embodiment 23. The method of any one of embodiments 1 to 22, wherein the primary human cell is a CD34+ hematopoietic stem and progenitor cell (HSPC), a T cell, a B cell, an airway basal stem cell, or a pluripotent stem cell (PSC).

Embodiment 24. The method of any one of embodiments 1 to 23, wherein the locus of interest is a gene selected from the group consisting of Hemoglobin Subunit Beta (HBB), C—C Motif Chemokine Receptor 5 (CCR5), Interleukin 2 Receptor Subunit Gamma (IL2RG), Hemoglobin Subunit Alpha 1 (HBA1), Stimulator Of Interferon Response cGAMP Interactor 1 (STING1) and Cystic Fibrosis Transmembrane Conductance Regulator (CFTR).

Embodiment 25. The method of any one of embodiments 1 to 24, wherein the frequency of HDR at the locus of interest in the cell is higher than the frequency in an equivalent cell in the presence of the SDN and homologous donor template, but in the absence of the DNA-PK inhibitor.

Embodiment 26. The method of embodiment 25, wherein the frequency of HDR at the locus of interest in the cell is at least about 10%, 20%, 30%, 40%, or more higher than the frequency in an equivalent cell in the presence of the SDN and homologous donor template, but in the absence of the DNA-PK inhibitor.

Embodiment 27. The method of any one of embodiments 12 to 26, wherein the sgRNA induces low to no indels at the locus of interest in the presence of the SDN but in the absence of the DNA-PK inhibitor.

Embodiment 28. The method of any one of embodiments 1 to 27, wherein the frequency of indels at the locus of interest in the cell is lower than the frequency in an equivalent cell in the presence of the SDN and homologous donor template, but in the absence of the DNA-PK inhibitor.

Embodiment 29. The method of embodiment 28, wherein the frequency of indels at the locus of interest in the cell is at least about 10%, 20%, 30%, 40%, 50%, 60%, 70%, or more lower than the frequency in an equivalent cell in the presence of the SDN and homologous donor template, but in the absence of the DNA-PK inhibitor.

Embodiment 30. The method of any one of embodiments 1 to 29, further comprising introducing a second SDN into the cell targeted to a second cleavage site at a second genetic locus, and introducing a second homologous donor template into the cell comprising a nucleotide sequence that is homologous to the second genetic locus,
wherein the second SDN cleaves the second genetic locus at the second cleavage site, and the second homologous donor template is integrated at the site of the cleaved second locus by HDR.

Embodiment 31. The method of embodiment 30, wherein the frequency of HDR is higher at both the locus of interest and the second genetic locus in the presence of the DNA-PK inhibitor than in the absence of the DNA-PK inhibitor.

Embodiment 32. The method of embodiment 30 or 31, wherein the frequency of indels is lower at both the locus of interest and the second genetic locus in the presence of the DNA-PK inhibitor than in the absence of the DNA-PK inhibitor.

Embodiment 33. A method of treating a genetic disorder in a human subject in need thereof, the method comprising:
providing an isolated primary cell from the subject;
genetically modifying the primary cell using the method of any one of embodiments 1 to 32, wherein the integration of the homologous donor template at the locus of interest in the cell corrects a mutation at the locus or leads to the expression of a therapeutic protein in the cell that is absent or deficient in the subject; and
reintroducing the genetically modified cell into the subject.

Embodiment 34. The method of embodiment 33, wherein the genetic disorder is 3-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof.

What is claimed is:

1. A method of genetically modifying a primary human cell, the method comprising:
(i) introducing into the cell a site-directed nuclease (SDN) targeted to a cleavage site at a genetic locus of interest;
(ii) introducing a homologous donor template into the cell, wherein the homologous donor template comprises a nucleotide sequence that is homologous to the locus of interest; and
(iii) introducing a DNA-PK inhibitor into the cell,
wherein the site-directed nuclease cleaves the locus at the cleavage site, and the homologous donor template is integrated at the site of the cleaved locus by homology directed repair (HDR).

2. The method of claim 1, wherein the DNA-PK inhibitor is a compound represented by the following formula: 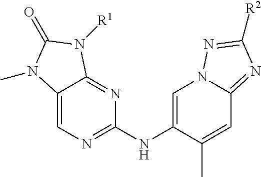
wherein:
R¹ is a cyclohexyl, tetrahydrofuranyl or oxanyl ring, each of which is optionally substituted by one or more groups selected from hydroxyl, methoxy, and methyl; and
R² is hydrogen or methyl,
or a pharmaceutically acceptable salt thereof.

3. The method of claim 2, wherein R¹ is oxanyl.

4. The method of claim 3, wherein R¹ is oxan-4-yl.

5. The method of claim 2, wherein R² is hydrogen.

6. The method of claim 2, wherein the DNA-PK inhibitor is AZD7648 represented by the following formula: 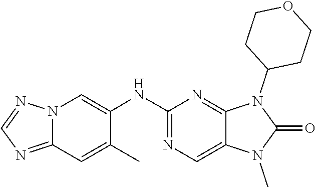 [SMILES: Cc1cc2ncnn2cc1Nc1ncc2c(n1)n(C1CCOCC1)c(=O)n2C]
or a pharmaceutically acceptable salt thereof.

7. The method of claim 1, wherein the DNA-PK inhibitor is VX984 represented by the following formula: 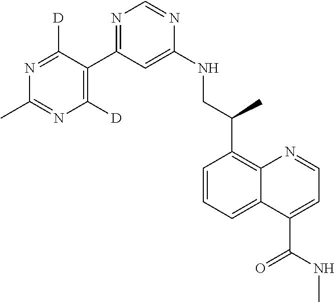
or a pharmaceutically acceptable salt thereof.

8. The method of claim 1, wherein the DNA-PK inhibitor is BAY8400 represented by the following formula: 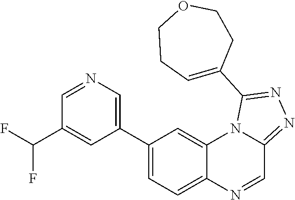 [SMILES: FC(F)c1cncc(-c2ccc3ncc4nnc(C5=CCCOCC5)n4c3c2)c1]
or a pharmaceutically acceptable salt thereof.

9. The method of claim 1, wherein the DNA-PK inhibitor has very high specificity for the catalytic subunit of DNA-PK (DNA-PKcs).

10. The method of claim 9, wherein the DNA-PK inhibitor with very high specificity for DNA-PKcs has an IC50 in the range of about 40 nM to about 1 μM for DNA-PKcs and an IC50 of greater than 1 μM for other PIKK family kinases.

11. The method of claim 10, wherein the other PIKK family kinases are ATM, ATR, PI3Kα, PI3Kβ, PI3Kγ, PI3Kδ, and/or mTOR.

12. The method of claim 1, wherein the SDN is an RNA-guided nuclease and the method further comprises introducing into the cell a single guide RNA (sgRNA) targeting the cleavage site, wherein the sgRNA directs the RNA-guided nuclease to the cleavage site.

13. The method of claim 12, wherein the sgRNA comprises 2′-O-methyl-3′-phosphorothioate (MS) modifications at one or more nucleotides.

14. The method of claim 13, wherein the MS modifications are present at the terminal nucleotides of the 5′ and 3′ ends.

15. The method of claim 12, wherein the RNA-guided nuclease is Cas9.

16. The method of claim 12, wherein the sgRNA and RNA-guided nuclease are introduced into the cell as a ribonucleoprotein (RNP).

17. The method of claim 16, wherein the RNP is introduced into the cell by electroporation.

18. The method of claim 12, wherein the sgRNA is introduced into cells at a concentration of less than about 150 μg/ml, 75 μg/ml, 30 μg/ml, or 15 μg/ml.

19. The method of claim 12, wherein the RNA-guided nuclease is introduced into cells at a concentration of less than about 300 μg/ml, 150 μg/ml, 60 μg/ml, or 30 μg/ml.

20. The method of claim 1, wherein the homologous repair template is introduced into the cell using an adeno-associated virus serotype 6 (AAV6) vector.

21. The method of claim 20, wherein the AAV6 vector is transduced into the cell at a multiplicity of infection (MOI) of less than about 2500, 1000, or 500.

22. The method of claim 21, wherein the MOI is about 500.

23. The method of claim 1, wherein the primary human cell is a CD34⁺ hematopoietic stem and progenitor cell (HSPC), a T cell, a B cell, an airway basal stem cell, or a pluripotent stem cell (PSC).

24. The method of claim 1, wherein the locus of interest is a gene selected from the group consisting of Hemoglobin Subunit Beta (HBB), C—C Motif Chemokine Receptor 5 (CCR5), Interleukin 2 Receptor Subunit Gamma (IL2RG), Hemoglobin Subunit Alpha 1 (HBA1), Stimulator Of Interferon Response cGAMP Interactor 1 (STING1) and Cystic Fibrosis Transmembrane Conductance Regulator (CFTR).

25. The method of claim 1, wherein the frequency of HDR at the locus of interest in the cell is higher than the frequency in an equivalent cell in the presence of the SDN and homologous donor template, but in the absence of the DNA-PK inhibitor.

26. The method of claim 25, wherein the frequency of HDR at the locus of interest in the cell is at least about 10%, 20%, 30%, 40%, or more higher than the frequency in an equivalent cell in the presence of the SDN and homologous donor template, but in the absence of the DNA-PK inhibitor.

27. The method of claim 12, wherein the sgRNA induces low to no indels at the locus of interest in the presence of the SDN but in the absence of the DNA-PK inhibitor.

28. The method of claim 1, wherein the frequency of indels at the locus of interest in the cell is lower than the frequency in an equivalent cell in the presence of the SDN and homologous donor template, but in the absence of the DNA-PK inhibitor.

29. The method of claim 28, wherein the frequency of indels at the locus of interest in the cell is at least about 10%, 20%, 30%, 40%, 50%, 60%, 70%, or more lower than the frequency in an equivalent cell in the presence of the SDN and homologous donor template, but in the absence of the DNA-PK inhibitor.

30. The method of claim 1, further comprising introducing a second SDN into the cell targeted to a second cleavage site at a second genetic locus, and introducing a second homologous donor template into the cell comprising a nucleotide sequence that is homologous to the second genetic locus,
wherein the second SDN cleaves the second genetic locus at the second cleavage site, and the second homologous donor template is integrated at the site of the cleaved second locus by HDR.

31. The method of claim 30, wherein the frequency of HDR is higher at both the locus of interest and the second genetic locus in the presence of the DNA-PK inhibitor than in the absence of the DNA-PK inhibitor.

32. The method of claim 30 or 31, wherein the frequency of indels is lower at both the locus of interest and the second genetic locus in the presence of the DNA-PK inhibitor than in the absence of the DNA-PK inhibitor.

33. A method of treating a genetic disorder in a human subject in need thereof, the method comprising:
providing an isolated primary cell from the subject;
genetically modifying the primary cell using the method of any one of claims 1 to 32, wherein the integration of the homologous donor template at the locus of interest in the cell corrects a mutation at the locus or leads to the expression of a therapeutic protein in the cell that is absent or deficient in the subject; and
reintroducing the genetically modified cell into the subject.

34. The method of claim 33, wherein the genetic disorder is β-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof.
